(19) United States
(12) Patent Application Publication
(10) Pub. No.: US 2026/0096994 A1
(43) Pub. Date: Apr. 9, 2026

(54) SOLID DISPERSIONS HAVING STABILIZED EMULSION PARTICLES

(71) Applicant: Boehringer Ingelheim International GmbH, Ingelheim am Rhein (DE)

(72) Inventors: Michael BRAUN, Senden (DE); Tao ZHANG, New Rochelle, NY (US)

(21) Appl. No.: 19/348,878

(22) Filed: Oct. 3, 2025

Related U.S. Application Data
(60) Provisional application No. 63/704,064, filed on Oct. 7, 2024.
(60) Provisional application No. 63/707,964, filed on Oct. 16, 2024.

(30) Foreign Application Priority Data
Jan. 30, 2025 (EP) 25155070.3

(51) Int. Cl. A61K 9/20; A61K 9/14; A61K 31/519

(52) U.S. Cl. CPC A61K 9/2095; A61K 9/146; A61K 9/2077; A61K 31/519

(57) ABSTRACT

Provided is a method to prepare a solid dispersion composition comprising an active pharmaceutical ingredient (API) and a pharmaceutically acceptable dispersion carrier, which includes (a) dissolving the API and the pharmaceutically acceptable dispersion carrier in a solvent to form a dissolved API/pharmaceutically acceptable dispersion carrier solution; and/or (b) combining the dissolved API/pharmaceutically acceptable dispersion carrier solution and an anti-solvent to form a combination, wherein the anti-solvent comprises a surfactant; and/or (c) mixing the combination to generate an emulsion with a predetermined mean particle size range; and/or (d) removing the solvent from the emulsion to generate the solid dispersion composition, wherein the solid dispersion composition has the predetermined mean particle size range. Preferably, the API in the solid dispersion composition is amorphous. Also provided is an emulsion and/or a solid dispersion composition comprising the API and a dispersion carrier. Also provided is a solid dispersion made by the methods of the invention. In embodiments, the present invention includes Compound (1), also known as zongertinib, and apparatus for continuous processing according to the methods disclosed herein.

### RELATED APPLICATION DISCLOSURE

This application claims the benefit of U.S. Provisional Pat. Appl. Nos. 63/704,064, filed Oct. 7, 2024, U.S. Provisional Pat. Appl. No. 63/707,964, filed Oct. 16, 2024, and also claims priority to European Application No. 25155070.3 filed Jan. 30, 2025, each and all of which is hereby incorporated by reference in its entirety.

### BACKGROUND OF THE INVENTION

Oral solid dosage forms, such as tablets and capsules, are widely preferred in pharmaceutical formulations due to their stability, ease of administration, and predictable release characteristics. These dosage forms offer controlled drug release, prolonged shelf life, enhanced stability, accurate dosing, patient convenience, and better patient compliance, making them an ideal choice for many medications.

Due to the increasing number of new chemical entities that exhibit poor solubility, low crystallinity, that are intrinsically amorphous, and/or have non-ideal crystal properties, among other challenges, various strategies have been applied to improve the bioavailability, such as amorphous solid dispersions, salts/cocrystal formation, complexation techniques, lipid-based delivery systems, nanoscale drug delivery systems and particle size reduction.

Amorphous solid dispersions (ASDs) offer a promising solution for enhancing the solubility and bioavailability of poorly water-soluble drugs. By formulating active pharmaceutical ingredients within a polymer matrix, ASDs enhance dissolution rates, leading to improved bio accessibility, pharmacokinetics, and potentially enhanced therapeutic efficacy. As of 2023, more than 30 US FDA-approved products containing an ASD have been commercialized, using spray drying, hot melt extrusion, and other manufacturing technologies for their production.

ASDs use dispersion carriers, most often polymer matrices, which stabilize the drug in the amorphous state, preventing drug recrystallization. This may offer control over drug release kinetics, and facilitate drug dissolution, and improve formulation properties such as compactibility. The common methods to prepare amorphous solid dispersions include spray drying and hot melt extrusions, where continuous solid dispersion is achieved by removal of the solvents or heat. The choice of the method depends on various factors such as the drug and polymer properties, formulation requirements, and scale of production.

Although conventionally manufactured solid dispersions are widely applied, controlling their physical properties, such as the particle morphology, particle size distribution, bulk density, and/or flowability, can be challenging. Additionally, the use of specialized techniques and equipment such as spray dryers to produce the solid dispersions or special thin-film evaporation equipment to densify the materials can increase manufacturing costs and complexity.

One challenge with solid dispersions is that when the co-precipitated material is spray-dried, the density can be low, e.g., challenging for tableting processes, and the morphology may be poor (e.g., fibrous). Common pharmaceutical unit operations downstream of spray-drying, such as roller compaction, are used to increase density and to improve flow properties of pharmaceutical powders, but add extra steps and often require additional excipients. These extra steps add, for example, extra costs, complexity, time for manufacturing and result in unfavorably large tablet sizes. For amorphous forms, these additional mechanical processing step(s) can result in recrystallization.

Anti-solvent precipitation is one process to prepare an active pharmaceutical ingredient (API) in a solid dispersion. The basic principle is that the API with or without a stabilizing agent, most commonly a polymer, is dissolved in a solvent; the solvent solution is then mixed with an anti-solvent (in which the API is insoluble). The API precipitates as a consequence of the change of supersaturation caused by mixing the solution and the anti-solvent. However, creating conditions where the particle size may be “tuned” to a predetermined particle size range and maintaining that particle size during subsequent processing steps, e.g., drying steps, has been challenging.

Therefore, there is the need to improve the material attributes of solid dispersions prepared from an anti-solvent precipitation process and to maintain the desired material attributes in downstream processing steps. This includes a need to improve the control over the material attributes during the process, e.g. via specific process conditions.

N-{1-[8-({3-methyl-4-[(1-methyl-1H-1,3-benzodiazol-5-yl)oxy]phenyl}amino)-[1,3]-diazino[5,4-d]pyrimidin-2-yl]piperidin-4-yl}prop-2-enamide, also herein referred to as compound (1) or zongertinib, is a HER2 (ErbB2) inhibitor described in WO 2021/213800, which is incorporated by reference herein in its entirety for all it teaches and discloses. Zongertinib is a potent and selective tyrosine kinase inhibitor of wild type and mutant HER2 that spares wild type epithelial growth factor receptor (EGFR). Therefore, it is useful for the treatment and/or prevention of diseases and/or conditions wherein the inhibition of wild type and/or mutant HER2 is of therapeutic benefit, especially oncological and/or hyperproliferative diseases, such as cancer.

As described in WO 2024/133289, which is incorporated by reference herein in its entirety for all it teaches and discloses, the solubility of compound (1) in aqueous media was found to be limited and strongly pH dependent with increased solubility at acidic conditions. This pH dependency is undesirable because it may decrease bioavailability and/or bioaccessibility of compound (1). WO 2024/133289 discloses a formulation of compound (1) as a solid dispersion, which provides consistent bioavailability and/or bioaccessibility and overcomes inter-patient stomach pH variability compared to the administration of formulations comprising compound (1) in crystalline form.

Proteolysis targeting chimeras (e.g. degraders including PROTACs) bind to proteins causing their degradation by inducing their ubiquitination. Degraders are tripartite or heterobifunctional molecules consisting of a part binding to the protein that is to be degraded, a second part that binds to and can artificially recruit an E3 ubiquitin ligase, and a linker that connects the two parts.

It would be desirable to find processes to improve the properties of the solid dispersions of APIs, including zongertinib and degraders, e.g., improve the resultant bulk material in order to simplify downstream processing of solid dispersions. Improvements to the properties of the bulk material include improved morphology, e.g. a unimodal particle size distribution with a tunable or controllable average particle size, an improved particle size distribution, increased flowability, and/or an increased bulk density. Such improvements are desired to produce homogeneous, dense and free flowing spheres, which are more accessible for direct compression into tablets.

### SUMMARY OF THE INVENTION

In one embodiment, the present invention includes a method to prepare a solid dispersion composition comprising an active pharmaceutical ingredient (API) and a pharmaceutically acceptable dispersion carrier, wherein the method comprises: (a) dissolving the API and the pharmaceutically acceptable dispersion carrier in a solvent to form a dissolved API/pharmaceutically acceptable dispersion carrier solution; (b) combining the dissolved API/pharmaceutically acceptable dispersion carrier solution and an anti-solvent to form a combination, wherein the anti-solvent comprises a surfactant; (c) mixing the combination to generate an emulsion with a pre-determined mean particle size range; and (d) removing the solvent from the emulsion to generate the solid dispersion composition, wherein the solid dispersion composition has the predetermined mean particle size range.

In one embodiment, the present invention includes a method to prepare a solid dispersion composition comprising an active pharmaceutical ingredient (API) and a pharmaceutically acceptable dispersion carrier, wherein the method comprises: (a) dissolving the API and the pharmaceutically acceptable dispersion carrier in a water immiscible solvent to form a dissolved API/pharmaceutically acceptable dispersion carrier solution; (b) combining the dissolved API/pharmaceutically acceptable dispersion carrier solution and a water miscible anti-solvent to form a combination, wherein the water miscible anti-solvent comprises a surfactant; (c) mixing the combination to generate an emulsion with a pre-determined mean particle size range; and (d) removing the water immiscible solvent from the emulsion to generate the solid dispersion composition, wherein the solid dispersion composition has the predetermined mean particle size range.

In embodiments, the API in the solid dispersion composition is amorphous.

In certain embodiments, the method further comprises washing the solid dispersion composition with the anti-solvent. In certain embodiments, the washing at least partially removes the surfactant. In some embodiments, the method is continuous, semi-continuous or batch.

In some embodiments, step (d) is performed in a thin film rotary evaporator.

In certain embodiments, the method further comprises removing the anti-solvent from the solid dispersion composition. In certain embodiments, the method further comprises tableting the solid dispersion composition.

In certain embodiments, the solid dispersion composition has a unimodal particle size distribution with a mean particle size range of about 5 to about 100 μm (microns) based on polarized light microscopy, and/or has a unimodal particle size distribution with a mean particle size range of about 20 to about 80 μm (microns) based on polarized light microscopy.

In certain embodiments, the solid dispersion composition has a unimodal particle size distribution with a mean particle size range of about 5 to about 100 μm (microns) based on polarized light microscopy, and/or has a unimodal particle size distribution with a mean particle size range of about 40 to about 80 μm (microns) based on polarized light microscopy.

In certain embodiments, the solid dispersion composition has a mean particle size range of about 20 to about 80 μm (preferably about 40 to about 80 μm) based on polarized light microscopy. In other words, the predetermined mean particle size range is of about 20 to about 80 μm (preferably about 40 to about 80 μm) based on polarized light microscopy.

In certain embodiments, the solid dispersion composition has an increased bulk density compared to a control solid dispersion composition, wherein the control solid dispersion composition is prepared according to the method as described herein and wherein the method omits step (b). In certain embodiments, the solid dispersion composition has a bulk density of about 0.2 to about 0.5 g/cm³ as measured by the ratio of the mass of an untapped sample of solid dispersion composition divided by its volume.

In certain embodiments, the solid dispersion composition has a flow function coefficient (FFc) of greater than 2, as measured by a powder rheometer, and/or the solid dispersion composition has an FFc of greater than 3, as measured by a powder rheometer.

In certain embodiments, the emulsion has a particle size growth of less than 20% at room temperature for at least 10 hours, and/or a particle size growth of less than 10% at room temperature for at least 24 hours.

In certain embodiments, the mixing comprises wet-milling with a tip rotational speed of between about 2000 to 5000 rpm, about 3000 to 5000 rpm, or about 3000 rpm (e.g., tip speed 4.7 to 7 m/s).

In certain embodiments, the pharmaceutically acceptable dispersion carrier is a polymer, and/or wherein the polymer is selected from the group consisting of hydroxypropyl methylcellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers; and/or wherein the polymer is hydroxypropyl methylcellulose acetate succinate (HPMCAS). In certain embodiments, the HPMCAS is present in amount of between 0.01-200 g per liter of solvent in step (a). In certain embodiments, the API is present in an amount between 1 and 100 g per L of solvent in step (a).

In certain embodiments, the solvent is water immiscible and the anti-solvent is water miscible.

In certain embodiments, the solvent comprises an alcohol, a ketone, an ester, dichloromethane (DCM), chloroform, tetrahydrofuran, acetonitrile, toluene, 1,1,1-trichloroethane or mixtures thereof. In certain embodiments, the anti-solvent comprises, consists of or consists essentially of water. In certain embodiments, the ratio of solvent:anti-solvent is between about 1:1 and 1:20 by volume.

In certain embodiments, the surfactant comprises or consists of polyvinyl alcohol (PVA) and/or the anti-solvent comprises or consists of polyvinyl alcohol (PVA) and water. In certain embodiments, the PVA is present in the anti-solvent in an amount of between about 0.05% and 10% of the anti-solvent (g/ml); and/or the PVA is present in the anti-solvent in an amount of between about 0.1% and 5% of the anti-solvent (g/ml). In certain embodiments, the PVA has a degree of hydrolysis of between approximately 85 and 95% and/or a viscosity of between approximately 7 and 9 mPa·s.

In certain embodiments, the API is a degrader (e.g. a PROTAC such as (E)-N-(f-(((5-(tert-butyl)oxazol-2-yl)methyl)thio)thiazol-2-yl)-1-(2-(4-(2-(2,6-dimethoxy-4-(3-oxo-3-(6-oxo-3,6-dihyropyridin-1-(2H)-yl)prop-1-en-1-yl)phenoxy)ethyl)piperazin-1-yl)-2-oxoethyl)iperidine-4-carboxamide trifluoroacetate). In certain embodiments, the API is compound (1)

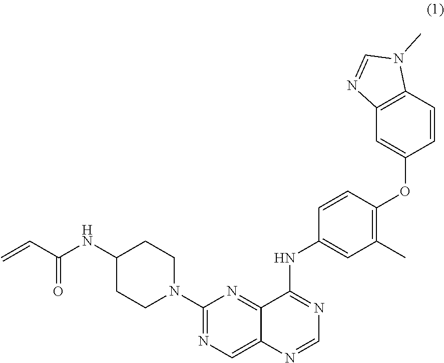 [SMILES: C=CC(=O)NC1CCN(c2ncc3ncnc(Nc4ccc(Oc5ccc6c(c5)ncn6C)c(C)c4)c3n2)CC1]

or a pharmaceutically acceptable salt thereof. In embodiments, when the API is Compound (1), the pharmaceutically acceptable dispersion carrier is HPMCAS and/or the solid dispersion composition comprises Compound (1) in an amount in a range of from 25 wt % to 75 wt %, and HPMCAS in an amount in a range of from 25 wt % to 75 wt %, based on a total weight of 100 wt % of the solid dispersion composition; and/or the weight ratio of compound (1): HPMCAS in the solid dispersion composition is of approximately 1:2; and/or the solvent is a mixture of dichloromethane and methanol. In embodiments, when the API is Compound (1), the solvent is about 85 parts by volume of dichloromethane to about 15 parts by volume of methanol; and/or the ratio of solvent:anti-solvent is about 0.2 by volume; and/or the PVA is present in the anti-solvent in an amount of between about 0.5% and 1.5% of the anti-solvent (g/ml); and/or the PVA is present in the anti-solvent in an amount of about 1% of the anti-solvent (g/ml); and/or the PVA has a degree of hydrolysis of between approximately 85 and 95%; and/or the PVA has a viscosity of between approximately 7 and 9 mPa·s.

In certain embodiments of the method, wherein the API is Compound (1), the solid dispersion composition has a bulk density of about 0.2 to about 0.5 g/cm³, as measured by the ratio of the mass of an untapped sample of solid dispersion composition divided by its volume; and/or the solid dispersion composition has a bulk density of about 0.3 to about 0.4 g/cm³ as measured by the ratio of the mass of an untapped sample of solid dispersion composition divided by its volume. In certain embodiments, wherein the API is Compound (1), the solid dispersion composition has a flow function coefficient (FFc) of greater than 2 as measured by a powder rheometer, and/or an FFc of greater than 3 as measured by a powder rheometer.

In certain embodiments of the method, wherein the API is Compound (1), the solid dispersion composition is characterized by having an x-ray powder diffractogram comprising no diffraction peak at 2-theta angles equal or below 40.0°, when measured at a temperature in the range of from 20 to 30° C. and with Cu-Kα radiation having a wavelength of 1.54056 Å or 1.54184 Å; and/or the solid dispersion composition is characterized by having a differential scanning calorimetry curve comprising a single glass transition temperature signal, when measured with modulated differential scanning calorimetry with a modulation amplitude of 1° C./min and a heating rate of 3.0° C./min; and/or the solid dispersion composition has a single glass transition temperature signal in the range of from 110-120° C.; and/or the emulsion has a particle size growth of less than 20% at room temperature for at least 10 hours and/or the emulsion has a particle size growth of less than 10% at room temperature for at least 24 hours.

In embodiments, the present invention includes an emulsion comprising an API, such as including Compound (1) or a pharmaceutically acceptable salt thereof, a pharmaceutically acceptable dispersion carrier, and a surfactant. In embodiments of the emulsion, the pharmaceutically acceptable dispersion carrier is hydroxypropyl methylcellulose acetate succinate; and/or compound (1) is amorphous; and/or the surfactant is polyvinyl alcohol; and/or compound (1) is present in an amount of about 20-30 wt %, based on a total weight of 100 wt % of the emulsion.

In embodiments, the present invention includes a solid dispersion composition comprising an API such as for example, comprising compound (1) or a pharmaceutically acceptable salt thereof, and a pharmaceutically acceptable dispersion carrier; wherein the solid dispersion composition has a bulk density of about 0.2 to about 0.5 g/cm³ as measured by the ratio of the mass of an untapped sample of solid dispersion composition divided by its volume and/or wherein the solid dispersion composition has a flow function coefficient (FFc) of greater than 2, as measured by a powder rheometer. In embodiments of the solid dispersion composition, the pharmaceutically acceptable dispersion carrier is hydroxypropyl methylcellulose acetate succinate; and/or Compound (1) is amorphous; and/or Compound (1) is present in an amount of about 20-30 wt %, based on a total weight of 100 wt % of the solid dispersion composition; and/or the solid dispersion composition has a bulk density of about 0.2 to about 0.5 g/cm³ as measured by the ratio of the mass of an untapped sample of solid dispersion composition divided by its volume; and/or the solid dispersion has a bulk density of about 0.3 to about 0.4 g/cm³ as measured by the ratio of the mass of an untapped sample of solid dispersion composition divided by its volume. In embodiments, the solid dispersion composition has a flow function coefficient (FFc) of greater than 2 as measured by powder rheometry and/or the solid dispersion composition has a flow function coefficient (FFc) greater than 3, as measured by powder rheometry.

In embodiments, the present invention also includes a solid dispersion composition comprising an API, such as for example, Compound (1), obtainable by a method comprising the steps of (a) dissolving Compound (1) or a pharmaceutically acceptable salt thereof and a pharmaceutically acceptable dispersion carrier in a solvent to form a solution; (b) combining the solution and an anti-solvent to form a combination, wherein the anti-solvent comprises a surfactant; (c) mixing the combination to generate an emulsion with a pre-determined mean particle size range; and (d) removing the solvent from the emulsion to generate the solid dispersion composition, wherein the solid dispersion composition has the predetermined mean particle size range. In embodiments of the solid dispersion composition obtainable by the method, the pharmaceutically acceptable dispersion carrier is hydroxypropyl methylcellulose acetate succinate; and/or the solvent comprises dichloromethane/methanol; and/or the anti-solvent comprises water; and/or the surfactant comprises polyvinyl alcohol; and/or Compound (1) is amorphous; and/or Compound (1) is present in an amount of about 20-30 wt %, based on a total weight of 100 wt % of the solid dispersion composition; and/or wherein the solid dispersion composition has a bulk density of about 0.2 to about 0.5 g/cm³ as measured by the ratio of the mass of an untapped sample of solid dispersion composition divided by its volume or has a bulk density of about 0.3 to about 0.4 g/cm³ as measured by the ratio of the mass of an untapped sample of solid dispersion composition divided by its volume. In embodiments, the solid dispersion composition has a flow function coefficient (FFc) of greater than 2 as measured by powder rheometry and/or the solid dispersion composition has a flow function coefficient (FFc) of greater than 3, as measured by powder rheometry.

In embodiments, the present invention also includes an apparatus for conducting a continuous or semi-continuous production of a solid dispersion composition of the invention. The apparatus may include (a) a container (300, 10, 110) comprising a dissolved API/pharmaceutically acceptable dispersion carrier solution in a solvent; (b) a container (400, 20, 120) comprising an anti-solvent/surfactant solution; (c) a mixing device (320, 28, 128) fluidly connected to the containers of (a) and of (b), and configured to mix the solutions in the containers (a) and (b) to generate an emulsion, wherein the emulsion has a pre-determined mean particle size range; (d) at least one solvent removal device (36, 326, 136, 156) fluidically connected to the mixing device (320, 28, 128) wherein the at least one solvent removal device (36, 326, 136, 156) is configured to remove the solvent from the emulsion to produce the solid dispersion composition; and (e) a decanter or filtration device (342, 206) fluidically connected downstream of the at least one solvent removal device (36, 326, 136, 156) configured to collect solid dispersion composition, wherein the decanter or filtration device (342, 206) is optionally configured to wash the collected solid dispersion composition with an anti-solvent. The apparatus may further comprise one or more fluidically connected pumps (404, 304, 24, 14, 124, 114, 324, 34) configured to deliver the solutions of (a) and (b) to the mixing device (320, 28, 128) and/or deliver the emulsion to the at least one solvent removal device (36, 326, 136, 156). In some embodiments, the at least one solvent removal device (326, 136, 156) is a thin film evaporator. In embodiment of the apparatus, the API is compound (1). In embodiments of the apparatus, wherein the API is compound (1), the pharmaceutically acceptable dispersion carrier is HPMCAS and optionally the weight ratio of compound (1): HPMCAS in the solid dispersion composition is of 1:2. In embodiments of the apparatus, wherein the API is compound (1), the solvent is a mixture of dichloromethane and methanol, preferably with a ratio of 85 parts dichloromethane to 15 parts methanol v/v. In embodiments of the apparatus, wherein the API is compound (1), the anti-solvent is water and/or wherein the ratio of solvent:anti-solvent is 0.2 by volume. In embodiments of the apparatus, wherein the API is compound (1), the surfactant is polyvinyl alcohol (PVA) and optionally the PVA is present in the anti-solvent in an amount of between 0.5% and 1.5% of the anti-solvent (g/ml), and optionally the PVA has a degree of hydrolysis of between approximately 85 and 95%; and/or the PVA has a viscosity of between approximately 7 and 9 mPa·s.

### BRIEF DESCRIPTION OF DRAWINGS

FIG. 1 shows a flow chart for the process conditions of the method of the invention. An API and HPMCAS-M are dissolved in DCM and MeOH. PVA is dissolved in H₂O. The emulsion is generated by simultaneously charging both solutions into a high shear mixing device using two pumps (not shown in FIG. 1). The resulting emulsion is then subject to vacuum distillation to remove the organic solvents. The solids are recovered by filtration, followed by washing and drying.

FIG. 2A shows a depiction of the equipment set up. Both an API and a polymer are dissolved in a reactor containing DCM and MeOH. PVA is dissolved in a separate reactor containing H₂O. Both solutions are charged into a high shear mixing device using pumps. The generated emulsion is collected in a tank for further distillation.

FIG. 2B shows an alternative depiction of the equipment set up. An API and a polymer are dissolved in a reactor containing solvent(s). A surfactant is dissolved in a separate reactor containing water. Both solutions are charged into an emulsifier via pumps. The generated emulsion is collected in a tank and further distilled to remove solvents and collect solids.

FIG. 3 shows emulsion stability vs. polyvinyl alcohol content for the emulsion of the invention. The emulsion exhibits increased emulsion stability compared to the emulsion without the presence of PVA. The higher PVA content results in longer kinetic stability of the emulsion. For example, the emulsion is stable for over 30 h with 1 wt % PVA in H₂O, but undergoes coalescence with 0% of PVA under the testing condition.

FIG. 4 shows resultant particle size D₉₀ of the dried solids vs. wet milling speed (RPM). The particle size can be controlled by changing the mixing intensity in the high shear mixing device. The higher mixing intensity results in lower particle size. The D₉₀ of the dry solids decreases from 69.2 μm to 21.4 μm when increasing the wet-milling speed from 3000 rpm to 4500 μm under testing condition.

FIG. 5 shows the XPRD pattern of the solid dispersion of Example 1, as well as two other solid dispersions made with different proportions of HPMC-AS and API. The XRPD of the solid dispersion compositions exhibited a lack of sharp diffraction peaks, indicating the solid dispersion compositions comprise the amorphous form of compound (1).

FIG. 6 shows the DSC of the solid dispersion composition of Example 1. The solid dispersion compositions show a T_{g} of 113° C.

FIG. 7 shows the particle size distribution (PSD) of the solid dispersion composition of Example 1. The D₁₀, D₅₀, and D₉₀ of the solid dispersion compositions are 13.7, 31.6, and 69.2 μm, respectively, measured by a Malvern Mastersizer 3000+ system. Heptane with 1 wt. % Lecithin was used as dispersant. The suspension was stirred at 3500 rpm during the measurement.

FIG. 8 shows drug release (n=3) of Example 1 solid dispersion (left curve) and Example 6 solid dispersion (right curve). Error bars represent the standard deviation from triplicate.

FIG. 9 shows a comparison of the FFc of conventional spray-dried Compound (1) (“SDD”), material made in Example 1, unblended (“CAPS”) and of blended Example 1 (“Blend”). The spray-dried bulk material processes a FFc of <1, indicating the material is not flowing. The CAPS material (solid dispersion composition) possesses an average FFc of 3.47 and is considered cohesive. Post blending, the blend with the solid dispersion composition shows an average FFc of 5.5, indicating the blend is easy-flowing.

FIG. 10 shows the XRPD pattern of the samples before (top) and after (bottom) an accelerated stability test. The XRPD of the solid dispersion composition after accelerated stability tests shows no sharp diffraction peaks, indicating the solid dispersion remains as amorphous after the stability test.

FIG. 11 shows bioaccessibility profiles for Example 6 tablet compared to Example 8 tablet in Fed State as cumulative release (%) (left panel) and mg released (right panel) per timepoint.

FIG. 12A shows concentration time profile in donors (dissolution) of crystalline API and formulated tablets from Example 6 and Example 8 in FaSSIFc. The top line shows the tablets of Example 6, the middle line shows tablets of Example 8, and the bottom line shows crystalline API. Error bars represent the standard deviation from triplicate.

FIG. 12B shows concentration time profile in receivers (permeation) of crystalline API and formulated tablets from Example 6 and Example 8 in FaSSIFc. The top line shows the tablets of Example 6, the middle line shows tablets of Example 8, and the bottom line shows crystalline API. Error bars represent the standard deviation from triplicate.

FIG. 13A shows mean plasma concentration-time profiles (dose-normalized) for beagle dogs.

FIG. 13B shows mean plasma concentration-time profiles (dose-normalized) for beagle dogs.

FIGS. 14A and 14B shows individual dog plasma concentration-time profiles (dose-normalized) for beagle dogs.

FIG. 15 shows polarized light microscope images of Panel a) Emulsion of Example 1; Panel b) Emulsion of Example 1 during solvent removal step (distillation), Panel c) solids of Example 1 after water wash (filtration), Panel d) solids after removal of the anti-solvent (vacuum-drying)(dry solids).

FIG. 16 shows a depiction of an apparatus set up for continuous or semi-continuous manufacturing of a solid dispersion composition, showing two tanks, one tank comprising solvent, pharmaceutically acceptable dispersion carrier and API; and the other tank comprising anti-solvent and surfactant, which are fluidically connected to a wet mill or a static mixer with continuous feeding to create an emulsion. Emulsion is then transferred to a thin film evaporator allowing for continuous solvent evaporation with solid dispersion composition collection. The solid dispersion composition may then be filtered and or decanted to remove solvent. Decanted/filtered solid dispersion can then be further washed with water and dried in a dryer and then optionally sieved.

FIG. 17 shows a depiction of an apparatus set up for continuous or semi-continuous manufacturing of a solid dispersion composition, showing two tanks, one tank comprising solvent, pharmaceutically acceptable dispersion carrier and API; and the other tank comprising anti-solvent and surfactant, which are fluidically connected to a wet mill to with continuous feeding to create an emulsion. Emulsion is then transferred to a distillation column allowing for continuous solvent evaporation with solid dispersion composition collection.

FIG. 18 shows a depiction of an apparatus set up for continuous or semi-continuous manufacturing of a solid dispersion composition, with capacity for in-line cleaning of thin film evaporators during the manufacturing process. The set up has two agitated tanks, one tank comprising solvent, pharmaceutically acceptable dispersion carrier and API; and the other tank comprising anti-solvent and surfactant, which are fluidically connected to a wet mill to with continuous feeding to create an emulsion. Emulsion is then transferred to one of two thin-film evaporators, allowing for continuous solvent evaporation of the emulsion. The solid dispersion composition (e.g., the evaporated emulsion) is then collected and rinsed with water, prior to drying, sieving, and packaging. The thin-film evaporator not in use may be cleaned using in-line cleaning solution.

FIG. 19(A) shows in-line analysis techniques as described in Example 2 during distillation: Blaze.

FIG. 19(B) shows in-line analysis techniques as described in Example 2 during distillation: Raman.

FIG. 19(C) shows a Raman spectrum of the emulsion as described in Example 2 prior to distillation.

FIG. 20A shows scanning electron microscope images of the solid dispersion composition of Example 1 at magnification: 500 um, as described in Example 23.

FIG. 20B shows scanning electron microscope images of the solid dispersion composition of Example 1 at magnification: 100 um, as described in Example 23.

FIG. 20C shows scanning electron microscope images of the solid dispersion composition of Example 1 at magnification: 50.0 um, as described in Example 23.

FIG. 21 A shows tabletability of the solid dispersion of Example 1 and the formulation of Example 6.

FIG. 21B shows compressibility of the solid dispersion of Example 1 and the formulation of Example 6.

FIG. 21C shows compactibility of the solid dispersion of Example 1 and the formulation of Example 6.

### DETAILED DESCRIPTION OF THE INVENTION

The present invention provides a method to prepare a solid dispersion composition, using a controlled API/polymer solidification strategy, to produce a solid dispersion composition in which the API is optionally in an amorphous form. In this method, an anti-solvent (in particular a water miscible anti-solvent) is used which contains at least one surfactant, to co-process APIs, especially poorly soluble APIs, with dispersion carriers, e.g. HPMCAS, into an emulsion, preferably a stabilized emulsion. Unexpectedly, the present inventors have found that during the anti-solvent precipitation process to prepare a solid dispersion composition, adding a surfactant to the anti-solvent and generating an emulsion produces solid dispersion compositions with an improved morphology, generating spherical particles of tunable or controllable average particle size with an improved particle size distribution. Specific particle average size and distribution can be controlled using different conditions to generate the emulsion, in particular shear conditions. Importantly, the resultant particles are in the form of an emulsion (for example, an oil in water (O/W)-type emulsion) that retains its stability during subsequent steps, including solidification/drying steps, resulting in a solid dispersion composition where the particle morphology (e.g. spherical shape) remains. The emulsion retains stability for a tunable period of time such that during a solvent removal step and thereafter, the spherical particulate morphology remains. Upon solidification/drying the emulsion, the spherical particles may shrink somewhat in particle diameter due to the loss of the solvent, but retain their morphology (e.g., spherical particles) during drying and or storage, with a predictable mean particle size and distribution. Improvements to the properties of the resultant bulk material of the solid dispersion composition include increased flowability, and an increased bulk density and/or tapped density. Such improvements produce homogeneous, dense and free flowing spheres, which are more accessible for direct compression into tablets or filling of hard capsules. Advantageously, the process of the invention is versatile and can be employed to prepare solid dispersion compositions with different APIs.

In instances where a word encompasses both a singular and plural meaning, the word may appear with a terminal “(s)”, e.g., “one, two or three stabilizing excipient(s).”

The term “material attributes” (also referred to herein as “material properties”) of a co-precipitated material refers to product properties of a solid dispersion composition of the invention including, but not limited to, bulk density, dissolution rate, solubility, flowability, compressive strength and particle size, which have influence on the ability to manufacture oral dosage forms and dictate pharmacokinetic behavior of the pharmaceutical in a subject of interest.

The process of the invention herein results in improved material attributes during and subsequent to solvent removal of the solid dispersion compositions of the invention, and thus optionally remove the need for additional manufacturing steps from a process train to achieve material attributes amenable to tablet compression, e.g., to avoid additional unit operations like densification steps such as roller compaction, and the like. The solid dispersion compositions of all embodiments show superior physical bulk properties, excellent solid state and chemical stability and have demonstrated similar in-vitro and in-vivo performance compared to solid dispersion compositions, that have been conventionally produced using spray-drying.

Additional abbreviations and acronyms used herein are defined as follows: w/w is weight for weight; wt. % or w.% is weight percent; g/cc or g/cm³ is gram per cubic centimeter; “i.e.” is that is; and “e.g.” is for example. Active pharmaceutical ingredient or “API” is used interchangeably with “drug.”

As used herein, the terms “approximately” and “about” mean within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range. When values are stated herein and are not preceded by the term “approximately” or “about”, such term is meant to be implied.

In an embodiment, the present invention includes (e.g., comprises, consists of, or consists essentially of) a method to prepare a solid dispersion composition comprising an active pharmaceutical ingredient (API).

The method includes the following steps: optionally, (a) dissolving the API and pharmaceutically acceptable dispersion carrier in a solvent (in particular a water immiscible solvent) to form a dissolved API/pharmaceutically acceptable dispersion carrier solution; optionally, (b) combining the dissolved API/pharmaceutically acceptable dispersion carrier solution and an anti-solvent (in particular a water miscible anti-solvent) to form a combination, wherein the anti-solvent comprises a surfactant; optionally, (c), mixing (e.g. wet-milling or exposure to a high-shear environment) the combination under conditions to generate an emulsion and/or a solid dispersion with a pre-determined mean particle size range; and optionally, (d), removing the solvent (in particular the water immiscible solvent) from the emulsion to generate a solid dispersion composition with the predetermined mean particle size range.

Some steps of the methods for making the solid dispersion compositions of the invention can generally utilize known processes as known in the art, for example as disclosed in S. V. Bhujbal et al., Acta PharmaceuticaSinica B 2021; 11(8):2505-2536, which is herein incorporated by reference. As used herein, the term “solid dispersion” refers to a system in a solid state comprising at least two components, wherein one component, such as compound (1) or generally an active pharmaceutical ingredient (API), preferably in amorphous state, is dispersed throughout another component such as a pharmaceutically acceptable solid dispersion carrier, particularly a dispersion polymer. As used herein, the term “solid dispersion” may also be substituted by the term “solid dispersion composition”, “co-precipitate” or “co-precipitated material”.

By dispersing an active pharmaceutical ingredient (API), preferably on a molecular level, in a, for example polymeric, pharmaceutically acceptable dispersion carrier, an amorphous state can be maintained, even when exposed to elevated temperature and/or humidity conditions, and the solid dispersion composition can reliably provide the API in amorphous form. In embodiments, the API is amorphous. The preferred feature of the API being amorphous can be applied to any embodiment disclosed herein to provide further embodiments according to the invention, in particular, it can be applied to any embodiment of the solid dispersion composition (including embodiments about the identity of the pharmaceutically acceptable dispersion carrier, the amounts of the components of the solid dispersion composition, etc.), the pharmaceutical composition, the kits, the uses and the processes described herein.

The term “amorphous” as used herein refers to a condensed phase where molecules are randomly orientated and characterized by the absence of any microscopic order, with no diffraction peaks by XRPD; an amorphous solid system may be composed of a single chemical entity or may be a multi-component system containing, e.g., an API, polymer and other excipients, without stoichiometric composition. Amorphous solids generally possess crystal-like short range molecular arrangement, but no long-range order of molecular packing as found in crystalline solids. The solid-state form of a solid may be determined e.g. by x-ray powder diffraction (“XRPD”) or modulated differential scanning calorimetry (“mDSC”). As used herein, the term “amorphous solid dispersion composition” may be substituted by “co-precipitated amorphous dispersion” (cPAD).

Herein, an API is defined as any substance or mixture of substances intended to be used as an active ingredient of a drug product. An API can also be known as a drug substance. Such substances are intended to furnish pharmacological activity or other direct effect in the diagnosis, cure, mitigation, treatment, or prevention of disease or to affect the structure and function of the body. A suitable API for the present invention includes BCS class II/IV compounds which are, for example, intrinsically amorphous. A suitable API for the invention also includes crystalline compounds where the crystals have low solubility in aqueous media. Low solubility is typically defined as less than 0.1 mol/L in aqueous media or a dose number higher than 10. As known in the art, adequate solubility for oral absorption can be expected for low dose numbers while larger dose numbers are expected to present solubility-limited absorption. For example, a suitable API for the invention has a dose number higher than about 10, higher than about 20, higher than about 30, higher than about 40, higher than about 60, higher than about 80, higher than about 100 or up to about 200. Additional candidate API for the instant invention include APIs which are crystals with non-ideal properties, such as crystals that are sensitive to mechanical stress e.g. needle-shaped crystals.

In embodiments, the API has low solubility in aqueous media, such as water.

In embodiments, the API has a solubility of less than about 0.1 mol/L in aqueous media, such as water.

In embodiments, the API has a dose number higher than about 10, higher than about 20, higher than about 30, higher than about 40, higher than about 60, higher than about 80, higher than about 100 or up to about 200.

In embodiments, the API is amorphous in step (a), e.g., upon addition or shortly following after addition to the pharmaceutically acceptable dispersion carrier and the solvent (in particular the water immiscible solvent).

In embodiments, the API is crystalline in step (a), i.e. when it is added to the pharmaceutically acceptable dispersion carrier and the solvent (in particular the water immiscible solvent). In particular, such crystalline API may comprise needle-shaped crystals.

In embodiments, the API includes or is a proteolysis targeting chimera (degrader, including PROTAC). A degrader is a heterobifunctional molecule containing two active domains and a linker, thus comprising two covalently linked protein-binding molecules: one capable of engaging an E3 ubiquitin ligase, and another that binds to a target protein meant for degradation. However, due to their size and complexity, many degraders exist as intrinsically amorphous compounds.

In embodiments, the API includes or is a tyrosine kinase degrader.

In embodiments, the API includes or is zongertinib or a pharmaceutically acceptable salt thereof.

In embodiments, a solvent is used to dissolve the API. Preferably, the solvent is water immiscible and/or organic. Preferably, the solvent is water immiscible. Thus, any embodiment herein referring to a “solvent” preferably refers to a “water immiscible solvent”. As used herein “water immiscible” includes poorly miscible solvents and thus refers to all solvents that do not form or only partially form a homogeneous mixture with water and that tend to separate into distinct phases when mixed with water.

Examples of solvents include any solvent or mixture of solvents (in particular any water immiscible solvent or mixture of water immiscible solvents) where the API is capable of at least partial dissolution and at least partially dissolves. In preferred embodiments, the API is capable of full dissolution and fully dissolves. Examples of suitable solvents that can be used individually or as mixtures include alcohols, such as methanol (“MeOH”), ethanol (“EtOH”), n-propanol, isopropanol and butanol such as n-butanol, 2-butanol, isobutanol and tert-butanol; ketones, such as acetone, methyl ethyl ketone and methyl isobutyl ketone; esters, such as methyl acetate, ethyl acetate and propyl acetate, isopropyl acetate, n-butyl acetate and isobutyl acetate; and various other solvents, such as dichloromethane (DCM), chloroform, tetrahydrofuran, acetonitrile, toluene and 1,1,1-trichloroethane. Preferably, the solvent referred to in any of the above-described processes and embodiments thereof is selected from the group consisting of, alcohols, ketones, esters, dichloromethane, chloroform, tetrahydrofuran, acetonitrile, toluene, 1,1,1-trichloroethane and mixtures thereof.

In one embodiment, said solvent is a mixture of dichloromethane (DCM) and methanol (MeOH). The relative amounts of DCM and MecOH in the mixture may vary. Preferably, the mixture comprises at least 10% v/v MecOH based on a total volume of 100% v/v of the mixture. In embodiments, the mixture comprises an excess of DCM. Still preferably, the weight:weight ratio of MecOH:DCM ranges from 5:95 to 95:5 (v/v). Preferably, MecOH and DCM are in a volume:volume ratio of approximately 10:90, 15: 85, 20:80, 25:75, or 30:70. A solvent mixture of MecOH:DCM in a ratio of approximately 15:85 (v/v) is used in an embodiment of the present invention.

As used herein, the term “dispersion carrier” refers to a carrier component that allows the API to be dispersed throughout such that a solid dispersion composition may form. In embodiments, the API is dispersed at the molecular level in the pharmaceutically acceptable dispersion carrier. Dispersion carriers appropriate for the invention are defined further herein.

The methods of the invention may include any pharmaceutically acceptable dispersion carrier suitable for use with the chosen API. In embodiments, the pharmaceutically acceptable dispersion carrier is a polymer. Therefore, the present invention provides a solid dispersion composition or a process to prepare it, wherein the solid dispersion composition comprises an API, in some embodiments, comprising compound (1) as defined herein or a pharmaceutically acceptable salt thereof; and a polymer. Polymeric dispersion carriers also are denoted “dispersion polymers”. Polymers are widely used in solid dispersion formulations. Different polymeric carriers lead to solid dispersions with various properties in terms of physical stability, phase behavior and drug release rate and extent. Due to the complex nature solid dispersion formulation carrier best suited for a given API need to be tested. The pharmaceutically acceptable dispersion polymer preferably is a neutral or acidic polymer.

In other embodiments, the pharmaceutically acceptable dispersion carrier is a polymer that is enteric or non-enteric, preferably enteric. The term “enteric polymer” refers to a pH-dependent acidic polymer that is insoluble or only slightly soluble at a low pH (e.g. about pH 1 up to but less than pH 3) but becomes soluble at a higher pH (e.g. pH 5 and above). In certain embodiments a pH-dependent polymer may become soluble at a pH range from about pH 5 and above, e.g. from about pH 6 to about pH 9, from about pH 6 to about pH 8, from about pH 5 to about pH 7, or from about pH 5 to about pH 6, which is generally less acidic than the gastric environment and roughly corresponds to pH values in the small intestine.

Examples of enteric polymers include but are not limited to methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate, HPMCAS), polyvinyl acetate phthalate (PVAP), methyl methacrylate-methacrylic acid copolymers (Eudragit™ L100), shellac, cellulose acetate trimellitate, sodium alginate and zein. The term “non-enteric polymer” refers to a neutral polymer that does not show pH-dependent solubility characteristics. Examples of non-enteric polymers include but are not limited to cellulose derivatives such as Methylcellulose (MC), ethylcellulose (EC), hydroxypropylcellulose (HPC), hydroxyethyl cellulose (HEC), hydroxypropyl methylcellulose (HPMC), poly-vinyl-pyrrolidone (PVP), copovidone such as polyvinylpyrrolidone-vinyl acetate copolymer (PVP-VA), poly (ethylene glycol) PEGs, starch derivatives like cyclodextrin,% Soluplus® which is an amphiphilic copolymer consisting of polyethylene glycol, polyvinyl caprolactam, and polyvinyl acetate.

In embodiments, the pharmaceutically acceptable dispersion carrier is a polymer, or more simply the polymer is, selected from the group consisting of hydroxypropyl methylcelluloses and esters thereof, polyvinylpyrrolidones and copolymers thereof, and polymethacrylates and copolymers thereof. The pharmaceutically acceptable dispersion carrier may contain a mixture of two or more polymers. Examples of such polymers include, but are not limited to, hydroxypropyl methylcellulose (and esters thereof, including acetate succinate (including each of HPMCAS-L, HPMCAS-M and HPMCAS-H), hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers.

Preferably, the hydroxypropyl methylcelluloses and esters thereof are selected from the group consisting of hydroxypropyl methyl cellulose acetate (HPMCA), hydroxypropyl methyl cellulose (HPMC), hydroxypropyl cellulose (HPC), methyl cellulose, hydroxyethyl methyl cellulose, hydroxyethyl cellulose, hydroxyethyl cellulose acetate, hydroxyethyl ethyl cellulose, hydroxypropyl methyl cellulose acetate succinate (HPMCAS), hydroxypropyl methyl cellulose phthalate (HPMCP), carboxymethyl ethyl cellulose (CMEC), cellulose acetate phthalate (CAP), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), and carboxymethylcellulose acetate butyrate (CMCAB). Preferably, the hydroxypropyl methylcelluloses and esters thereof are selected from the group consisting of hydroxypropyl methylcellulose acetate succinate and hydroxypropyl methylcellulose, in particular hot melt extrusion-grade hydroxypropyl methylcellulose.

Preferably, the polyvinylpyrrolidones and copolymers thereof are selected from the group consisting of polyvinylpyrrolidone vinyl acetate copolymer (PVP-VA), polyvinyl alcohols, polyvinyl alcohol polyvinyl acetate copolymers and polyvinylpyrrolidone (PVP). Polyvinylpyrrolidone (PVP), also is commonly denoted polyvidone or povidone. In embodiments, the polyvinylpyrrolidones and copolymers thereof are a polyvinylpyrrolidone vinyl acetate copolymer (PVP-VA).

Preferably, the polymethacrylates and copolymers thereof are selected from the group consisting of methacrylic acid-ethyl acrylate copolymer, methacrylic acid-methyl methacrylate copolymer, methyl methacrylate and methacrylic acid copolymer. Polymethacrylates and copolymers thereof are, for example, available under the brand name Eudragit® from Evonik Industries AG. Methacrylic acid-methyl methacrylate copolymer is, for example, available under the brand name Eudragit® L100. In certain embodiments, the polymethacrylates and copolymers thereof are a methylacrylic acid methyl methacrylate copolymer.

In particular embodiments, the pharmaceutically acceptable dispersion carrier is a polymer, or more simply the polymer is selected from the group of hydroxypropyl methylcellulose acetate succinate (HPMCAS), polyvinylpyrrolidone vinyl acetate copolymer (PVP-VA), methylacrylic acid methyl methacrylate copolymer (such as Eudragit® L100), and hot melt extrusion-grade hydroxypropyl methylcellulose (HPMC HME).

In certain embodiments, the pharmaceutically acceptable dispersion carrier is hydroxypropyl methylcellulose acetate succinate (HPMCAS). HPMCAS also is known as hypromellose acetate succinate.

Hypromellose acetate succinate (HPMCAS) can be obtained by introducing acetyl and succinoyl groups to the hydroxyl groups of the backbone of hydroxypropyl methylcellulose (HPMC) also known as hypromellose. This procedure can be carried out by known methods, for instance by treating HPMC with acetic anhydride and/or with succinic anhydride. Acetic anhydride and succinic anhydride can be reacted with hydroxypropyl methylcellulose (HPMC) under specifically controlled conditions to produce HPMCAS with varying extent of substitution of acetyl and succinoyl groups.

HPMCAS is available in several grades (L, M and H) varying in extent of substitution of acetyl and succinoyl groups, based on the content of acetyl and succinoyl groups (wt %) in the HPMCAS molecule. Any grade of HPMCAS is usable in the solid dispersion composition of the invention. Preferably, HPMCAS of grade L, M or H is used. In certain embodiments, the pharmaceutically acceptable dispersion carrier is HPMCAS grade L. In certain embodiments, the pharmaceutically acceptable dispersion carrier is HPMCAS grade M. HPMCAS grade M may comprise an acetyl content of 7-11 wt %; a succinoyl content of 10-14 wt %; methoxyl content of 21-25 wt %; and a hydroxypropoxy content of 5-9 wt %. Preferably, HPMCAS grade M (HPMCAS-M) is soluble at pH ≥6. In certain embodiments, the dispersion carrier is HPMCAS grade H. Preferably, granular HPMCAS (HPMCAS-G) is used. HPMCAS-G can be used for any grade of HPMCAS, in particular for grade M, such that HPMCAS-MG is used.

The ratio of API to total weight to solvent volume may be from about 0. 1 g/l to about 1000 g/l. In embodiments, the ratio of API/pharmaceutically acceptable dispersion carrier total weight to solvent volume is from about 1 g/l to about 500 g/l, from about 10 g/l to about 200 g/l, from about 20 g/l to about 100 g/l or from about 30 g/l to about 50 g/l. In embodiments, API to solvent is about 40 g/L. The solution can be mixed or treated in any method known in the art to form the API solution, ideally to achieve full dissolution.

The ratio of pharmaceutically acceptable dispersion carrier to total weight to solvent volume may be from about 0.1 g/l to about 1000 g/l. In embodiments, the ratio of API/pharmaceutically acceptable dispersion carrier total weight to solvent volume is from about 1 g/l to about 800 g/l, from about 20 g/l to about 400 g/l, from about 40 g/l to about 200 g/l or from about 60 g/l to about 100 g/l. In embodiments, pharmaceutically acceptable dispersion carrier to solvent is about 80 g/L. The solution can be mixed or treated in any method known in the art to form the pharmaceutically acceptable dispersion carrier solution, ideally to achieve full dissolution.

In embodiments, the method of the invention excludes a spray-drying step.

In embodiments, the method of the invention further includes combining the dissolved API/pharmaceutically acceptable dispersion carrier (in the solvent) and an anti-solvent, wherein the anti-solvent comprises a surfactant, to form a combination. The combining step may be made by any method known in the art, for example, introduction of separate streams of the anti-solvent, optionally containing the surfactant, and the solvent containing the dissolved API/pharmaceutically acceptable dispersion carrier using pumps, for example, into a common container. Order of addition and/or flow rates of each component can be adjusted as necessary to form a miscible solution. In embodiments, the anti-solvent with optional surfactant and solvent containing API/dispersion carrier may be added simultaneously into a container, such as, for example, a mixing container or mixing device. The simultaneous addition may be at the same rate or at different rates, optionally, timed such that the separate streams of each component will complete at similar time points in the method.

In embodiments, in step (b), the dissolved API/pharmaceutically acceptable dispersion carrier solution and the anti-solvent are combined in a high shear mixing device. A high shear mixing device may also be referred to as a homogenizer.

As used herein, the term “anti-solvent” refers to a mixture (preferably a solution) of a surfactant in a solvent where the API and/or pharmaceutically acceptable dispersion carrier is/are insoluble or poorly soluble, e.g. with a solubility of less than about 0.1 mol/L. In embodiments, the anti-solvent of the present invention comprises a solvent in which the API and pharmaceutically acceptable dispersion carrier solution is insoluble or poorly soluble. Expressed another way, the solvent and the anti-solvent may be immiscible or poorly miscible with another. In embodiments, the anti-solvent of the present invention comprises a solvent in which the API has a solubility of less than about 0.1 mol/L. In an embodiment, the anti-solvent is chosen to decrease the solubility of the API and pharmaceutically acceptable dispersion carrier solution. The solubility of the API and pharmaceutically acceptable dispersion carrier solution in the anti-solvent optionally, may be decreased by methods known in the art, such as, for example, adjusting the temperature of the anti-solvent and/or combination, and/or adding salts and/or buffers and/or in adjusting the pH (e.g., pH<6) of the anti-solvent to minimize API/pharmaceutically acceptable dispersion carrier solubility.

Preferably, the anti-solvent is water miscible. Thus, any embodiment herein referring to an “anti-solvent” preferably refers to a “water miscible anti-solvent”. “Water miscible” as used herein can be used to described water itself. In an embodiment, the anti-solvent is aqueous. In embodiments, the anti-solvent comprises water.

In the present invention, surfactants can stabilize the emulsions of the instant invention for a period of time in order to allow solvent removal from the emulsion to generate a solid dispersion composition.

Emulsions are considered thermodynamically unstable systems and stability as defined for the present invention includes kinetic emulsion stability over a certain period of time. An emulsion is a colloid of two or more immiscible liquids where one liquid contains a dispersion of the other liquid. During the mixing step, an emulsion is formed, e.g., comprising an aqueous continuous phase; and an organic dispersed phase in particle or droplet form, comprising API, the dispersion carrier, and the solvent. In the absence of the surfactant, the emulsion will break (e.g., coalescence and/or fusion of individual droplets, leading to larger observed droplet sizes). The emulsions of the present invention have increased particle size stability, as shown by e.g., reduced coalescence and/or fusion of droplets over a given period of time; or observed during subsequent processing steps such as solvent removal step(s).

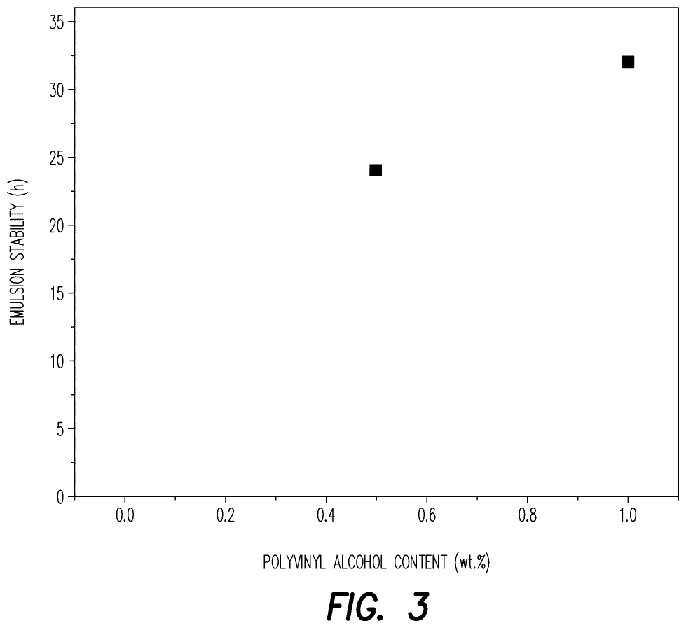

Increased particle size stability may be measured by methods known in the art, such as, for example, Polarized Light Microscopy (PLM). Reductions in the rate of particle diameter size growth may be shown by particle diameter size growth that is less than 5%, less than 10%, at least 15%, at least 20%, at least 25%, at least 30%, at least 35% over the time periods as defined herein. For example, emulsion stability increases with the addition of PVA as shown in FIG. 3, wherein at 0% wt. % PVA, emulsions are not stable, yet at 0.5 wt. % PVA, emulsions are stable for approximately 23 hours, and at 1% wt. % PVA, emulsions are stable for approximately 30 hours.

Stability may also be defined for the present invention as solid form stability, or the absence or minimization of recrystallization of the amorphous form of the API in the emulsion. Minimized recrystallization includes absence of the appearance of peaks via XRPD and/or no observable crystals via PLM for periods of time as defined herein or during subsequent processing step(s). The physical stability may be defined as physical stability of the amorphous form as indicated by lack of recrystallization under storage conditions, such as, for example, an accelerated stability study where samples are stored at 70° C. and 75% relative humidity, for periods that include one, two, three, four, six, eight or ten weeks. In embodiments, no recrystallization is observed under defined storage conditions of storage at 70° C. and 75% relative humidity for a period of about three weeks.

The emulsions of the present invention may be stable (e.g., have reduced rate of particle size diameter growth by set amounts described herein), for about 15 minutes or more, about 30 minutes or more, about 60 minutes or more, about 2 hours or more, about 4 hours or more, about 6 hours or more, about 8 hours or more, about 10 hours or more, about 18 hours or more, about 24 hours or more, about 30 hours or more, about 36 hours or more, about 48 hours or more, about 60 hours or more, up to about 168 hours. Alternatively, the emulsions of the present invention may be stable for about 30 minutes to 60 minutes, for about 60 minutes to about 2 hours, from about 2 hours to about 4 hours, from about 2 hours to about 6 hours, from about 2 hours to about 8 hours, from about 2 hours to about 10 hours, from about 4 hours to about 18 hours, from about 4 hours to about 24 hours, from about 8 hours to about 24 hours, from about 10 hours to about 24 hours, from about 18 hours to about 30 hours, from about 18 hours to about 36 hours, from about 24 hours to about 48 hours, or ranges therein between.

In an embodiment, a surfactant of the present invention can comprise any substance capable of stabilizing or enhancing stability of an emulsion of the present invention. In an embodiment, the surfactant is an emulsifier. An emulsifier can lower the interfacial surface tension between two liquids, thus increasing the kinetic stability of a mixture. Surfactants of the invention can include amphiphilic molecules, containing a hydrophilic moiety and a hydrophobic moiety. Without being bound by theory, the present inventors believe that a surfactant of the present invention may behave as an emulsifier forming a film over one phase in a mixture to form globules that repel each other, allowing them to remain evenly dispersed and not coalesce. Suitable surfactants of the invention include non-ionic surfactants. Non-ionic surfactants are surfactants that contain ether [—(CH₂CH₂O)ₙOH] and/or hydroxyl [—OH]hydrophilic groups. Unlike anionic and cationic surfactants, non-ionic surfactants are nonelectrolytes; that is, their hydrophilic groups do not ionize at any pH value. In embodiments, the surfactant is a non-ionic surfactant.

One example of a non-ionic surfactant suitable for the present invention is polyvinyl alcohol (PVA). PVA is a water-soluble synthetic polymer represented by the formula (C₂H₄O)ₙ. The value of n for commercially available materials is between 500 and 5000, which is roughly equivalent to a molecular weight range of 20,000 to 200,000. PVA is unique among the vinyl polymers in the fact that the monomer, vinyl alcohol, cannot exist in the free form. PVA is manufactured by the polymerization of vinyl acetate and then converted by a hydrolysis (alcoholysis) process. Various grades based on extent of hydrolysis exist with unconverted fractions being polyvinyl acetate. PVA is a well-known pharmaceutical polymer and is used in e.g. topical pharmaceutical and in ophthalmic formulations such as artificial tears and integrated into contact lenses for lubrication purposes. It has also been used in sustained-release formulations for oral administration and transdermal patches. Oral toxicity has been evaluated and PVA has been found safe even at high levels of consumption; the no-observed-adverse-effect level (NOAEL) for rats in a 3-month study was 5000 mg/kg body weight/day. In an embodiment, the surfactant of the invention is polyvinyl alcohol. Other examples of suitable non-ionic surfactants include polyvinylpyrrolidone (PVP), hydroxypropyl methylcellulose (HPMC), polyethylene glycol (PEG), carboxymethylcellulose (CMC), and polyacrylamide (PAM). Other examples of suitable non-ionic surfactants include sorbitan fatty acid esters, (Spans®), polysorbates (Tweens®), and poloxamer (Pluronics®). Sorbitan fatty acid esters such as sorbitan monopalmitate are oil-soluble emulsifiers that promote the formation of w/o emulsions. Polyethylene glycol sorbitan fatty acid esters (Tweens) are water-soluble emulsifiers that promote the formation of o/w emulsions. Pluronics are block copolymers of hydrophilic poly (oxyethylene) (POE) and hydrophobic poly(oxypropylene) (POP) represented by the general formula POEnPOPm-POEn, where n and m represent the number of OE and OP, respectively. The Spans and the Tweens come in different molecular weight or size ranges, which differ in their physical properties.

In embodiments, the surfactant is selected from the group consisting of: polyvinyl alcohol (PVA), polyvinylpyrrolidone (PVP), hydroxypropyl methylcellulose (HPMC), polyethylene glycol (PEG), carboxymethylcellulose (CMC), polyacrylamide (PAM), sorbitan fatty acid esters (Spans®), polysorbates (Tweens®), poloxamer (Pluronics®) and mixtures thereof.

In embodiments, the surfactant is PVA. Suitable PVA for the present invention includes PVA which is partially hydrolyzed. Partially hydrolyzed (e.g. partially acetylated) PVA retains (some) acetate groups which provides more amphiphilic character to the PVA, allowing better stabilization of the inventive emulsions. The degree of hydrolysis (mol-%) can vary between about 80% to about 98%, or about 82% to about 95%, or about 85% to about 90%. In some embodiments, the degree of hydrolysis can be in a range in between about 84% and about 94%, such as, for example, a range of 84% to 87%, or a range of 86% to 89%, or a range of 91% to 94%. In a preferred embodiment, the degree of hydrolysis can vary between about 85% to 90%, and in an even more preferred embodiment, the range of hydrolysis can be a range of 86% to 89%.

Additionally, suitable PVA for the invention includes PVA with particular viscosity ranges, which may also correspond to certain average molecular weights of the PVA product, as known in the art. For example, preferred viscosity ranges include between 3 mPa·s and about 20 mPa·s, or about 5 mPa·s to about 15 mPa·s, or about 7 mPa·s to about 9 mPa·s. In some embodiments, preferred viscosity ranges can include between about 3 and 5 mPa·s, between about 5 and 6 mPa·s, between about 7 and 9 mPa·s, between about 11 and 15 mPa·s, or between about 16 and 20 mPa·s. More preferred are viscosity ranges between about 7 and 9 mPa·s. All viscosity measurements refer to the viscosity of a 4% aqueous solution at 20° C., as measured by DIN 53015 viscometry.

Other suitable surfactants may be selected based on their similarity in function to PVA.

In embodiments, the anti-solvent comprises, consists essentially of or consists of PVA and water.

In embodiments, the ratio of solvent (the solvent comprising the dissolved API and pharmaceutically acceptable dispersion carrier) to the anti-solvent is about 1 parts solvent (by volume) to a volume of anti-solvent of between about 0.5 parts to about 50 parts. In embodiments, per 1 part volume of solvent, the volume of anti-solvent can be about 0.5 parts, about 1 part, about 2.5 parts, about 5 parts, about 10 parts, about 20 parts, or about 50 parts. Or, per 1 part volume of solvent, the volume of anti-solvent can be about 1 part to about 20 parts, about 2.5 parts to about 10 parts, or about 5 parts.

In embodiments, the surfactant (g) is present in the anti-solvent (ml) in an amount of about 0.05% g/ml; about 0.1% g/ml; about 0.15% g/ml; about 0.2% g/ml; about 0.25% g/ml; about 0.3% g/ml; about 0.35% g/ml; about 0.4% g/ml; about 0.5% g/ml; about 0.6% g/ml; about 0.7% g/ml; about 0.8% g/ml; about 0.9% g/ml; about 1% g/ml; about 1.1% g/ml; about 1.2% g/ml; about 1.3% g/ml; about 1.4% g/ml; about 1.5% g/ml; about 1.6% g/ml; about 1.7% g/ml; about 1.8% g/ml; about 2% g/ml; about 2.5% g/ml; about 3% g/ml; about 4% g/ml; about 4% g/ml; about 6% g/ml; about 7% g/ml; about 8% g/ml; or about 10% g/ml. The amount of surfactant can be determined by the length of time that stability as defined herein of the emulsion/solid dispersion composition is desired for the specific solvent removal methods, with higher concentrations of surfactant being correlated with greater stability.

In other embodiments, the surfactant is present in the anti-solvent in an amount of between about 0.1% g/ml to about 10% g/ml, or between about 0.2% g/ml to about 5% g/ml, or between about 0.3% g/ml to about 3% g/ml, or about between about 0.5% g/ml to about 2% g/ml, or about 0.7% g/ml to about 1.5% g/ml, or about 0.8% g/ml to about 1.2% g/ml.

In embodiments, the anti-solvent comprises, consists essentially of or consists of PVA and water, preferably in an amount of about 1 g of PVA per ml of water.

The method of the invention further includes a step of mixing the anti-solvent with optional surfactant, together with the dissolved API/pharmaceutically acceptable dispersion carrier to generate an emulsion. The mixing can take place according to any procedure known in the art to generate emulsions. High-shear mixing is preferred. High-shear conditions may be provided using a rotor-stator device or intense static mixing with an impeller, which serves as a simple and scalable method to prepare the emulsion with controllable droplet size.

In embodiments, the mixing is high-shear mixing. In embodiments, the mixing in step (c) is high-shear mixing.

In the present invention, a high shear sufficient to produce an emulsion according to the invention can be achieved via the use of high shear rotor-stator mixers, however, static mixers can also be used as long as sufficient shear can be produced.

As is known in the art, scaling of pharmaceutical high shear operations can be achieved (see e.g., Harter, et al., “High Shear Rotor-Stator Wet Milling for Drug Substances”, Org. Process Res. Dev. 2013, 17, 10, 1335-1344). Scaling can be based on constant tip speed (assuming similar shear gaps) as well as similar batch turnovers in recycle mode (e.g., pass number) to result in equivalent terminal particle size. “Tip speed” can be derived from the rotational speed, typically, 3000-4500 rpm, which corresponds to a tip speed of about 4.7 to 7 m/s, based on the rotor diameter of 30 mm. While using the constant tip speed approach, applying the concept of batch turnovers in recycle mode achieves a reasonable approximation of the time required to achieve the desired particle size for a given batch size. Batch turnovers is a means to represent the number of passes through the mill, as calculated by

$$batch turnover = \frac{flow rate through mill * time}{volume of slurry being milled}$$

In embodiments, the mixing takes place with a rotor-stator device, in particular a high shear rotor-stator device.

In embodiments, the rotor-stator configuration can be equivalent to use of a high shear mixer IKA® Magic Lab®, Wilmington NC), using three stages with rotor stator configuration of medium/coarse/coarse in an RPM range of 3000-4500 RPM.

The term “particle size distribution” as used herein refers to a list of values or a mathematical function that defines the relative amount, of particles present in a sample according to size. Particle size distribution can be characterized by one or more values, such as D₅₀, D₉₀ or D₁₀. The particle size distribution may be determined by means well known to the skilled artisan e.g. by laser diffraction. “D₉₀”, as used herein, describes the value of particle size at which 90% of the total counts of particles is comprised of particles no larger than the indicated size. “D₅₀”, as used herein, describes the value of particle size at which 50% of the total counts of particles is comprised of particles no larger than the indicated size. D₅₀ is also known as mean particle size of a distribution. “Mean particle size range” refers to a range or interval of mean particle size (e.g. about 5 to about 100 μm), as opposed to a specific value. “D₁₀”, as used herein, describes the value of particle size at which 10% of the total number of particles is comprised of particles no larger than the indicated size.

A particle size distribution may be pre-determined, meaning that it is established prior to performing the method according to the invention. In particular, a pre-determined mean particle size range can refer to a target interval of particle sizes (i.e. from x to y μm) that is selected in advance of performing the method according to the invention. The pre-determined mean particle size range may vary depending on desired properties of the composition, preferably it will be lower than about 100 μm, or lower than about 90 μm, or lower than about 80 μm, or lower than about 70 μm, or lower than about 60 μm, or lower than about 50 μm.

As guidance for achieving a predetermined or preselected particle size distribution, the present inventors have provided Example 3, in which the inventors found that using the IKA® Magic Lab®, at an RPM of 3000, a D₉₀ was achieved of about 70 μm (microns); at an RPM of 3750, a D₉₀ was achieved of about 37 μm (microns), and at an RPM of 4500, a D₉₀ was achieved of about 21 μm (microns). Higher RPM in general, will result in smaller size particles. The conditions can accordingly be adjusted to produce particles of the predetermined particle mean size, size range, particle D₁₀, D₅₀, and/or D₉₀. Optionally, particle size is determined via polarized light microscopy (PLM).

In one embodiment, the emulsion or solid dispersion composition comprises particles having a mean particle size range of about 5 to about 100 μm (microns), or about 10 to about 90 μm (microns), or about 15 to about 80 μm (microns), or about 20 to about 70 μm (microns), or about 30 to about 60 μm (microns), or about 40 to about 50 μm (microns), in particular when determined by laser diffraction or polarized light microscopy. In another embodiment, the mean particle size range is about 20 to about 80 μm (microns), in particular when determined by laser diffraction or polarized light microscopy. In another embodiment, the mean particle size range is about 40 to about 80 μm (microns), in particular when determined by laser diffraction or polarized light microscopy. In another embodiment, the mean particle size range is about 30 to about 50 μm (microns), in particular when determined by laser diffraction or polarized light microscopy. Preferably, in the embodiments of this paragraph, the API is zongertinib or a pharmaceutically acceptable salt thereof. Sizes may be determined for the emulsion or preferably, for the solid dispersion composition.

In embodiments, the particles have a D₉₀ value of at least 10 μm (microns), but not more than 100 μm, preferably of not more than 90 μm, most preferably of not more than 85 μm; and/or the particles have a D₅₀ value of at least 10 μm (microns), but not more than 50 μm, preferably of not more than 45 μm, most preferably of not more than 40 μm; and/or the particles have a D₁₀ value of at least 2, 4, 6, 10 or 15 μm (microns), but not more than 20 μm, preferably of not more than 15 μm, most preferably of not more than 13 μm, in particular when said D₉₀, D₅₀ or D₁₀ value is determined by laser diffraction or polarized light microscopy. Preferably, in the embodiments of this paragraph, the API is zongertinib or a pharmaceutically acceptable salt thereof.

In embodiments, the particles have a D₉₀ value in the range of from 50 to 100 μm, preferably of from 55 to 90 μm, most preferably of from 60 to 85 μm; and/or a D₅₀ value in the range of from 25 to 50 μm, preferably of from 30 to 45 μm, most preferably of from 30 to 40 μm; and/or a D₁₀ value in the range of from 1 to 20 μm, preferably of from 5 to 15 μm, most preferably of from 10 to 13 μm, in particular when said D₉₀, D₅₀ or D₁₀ value is determined by laser diffraction or polarized light microscopy. Preferably, in the embodiments of this paragraph, the API is zongertinib or a pharmaceutically acceptable salt thereof.

The methods of the invention also include a removal step, e.g., a step of removing the solvent from the emulsion to generate a solid dispersion composition. Various strategies can be employed to solidify the emulsion, including evaporating in a batch reactor under vacuum/rotary evaporator, or charging the emulsion into hot water solution, or evaporating at elevated temperature, or use of a multistage continuous mixed-suspension, mixed-product removal (MSMPR) reactors. Alternatively, the solvent may be removed by methods known in the art, e.g., vacuum distillation, falling film evaporation, thin film evaporation, rotary evaporation, desiccator, or air drying. The appropriate method for removing the solvent may be selected by the skilled person depending on the boiling point of the solvent and the anti-solvent, the sensitivity of the emulsion to heat, for example. As discussed hereinabove, one of the advantages of the present invention is that the surfactant(s) used in the present invention stabilize the emulsions of the instant invention for a period of time in orderto allow solvent removal from the emulsion to generate a solid dispersion composition with suitable stability as defined herein. The time frame for a stable emulsion to allow for suitable solvent removal is preferably stability for the entire process of solvent removal, e.g., the time periods as provided herein.

In one embodiment, the method of the invention further includes wherein the solid dispersion composition is washed with one or more anti-solvent(s). The wash step may be carried out with the same anti-solvent as used in the combining steps of the present invention, or the anti-solvent may be different. The wash step may be carried out by any method known in the art, for example, resuspending the solid dispersion composition in the one or more anti-solvents and collecting the solid dispersion composition, for example, by filtration or centrifugation. The wash step can be performed once or multiple times. In embodiments, the anti-solvent used for the washing step(s) consists of or consists essentially of purified water. In embodiments, the anti-solvent used for the washing step(s) omits surfactant.

In embodiments, the wash step can at least partially remove the one or more surfactants from the solid dispersion composition. In embodiments, the amount of surfactant remaining in the solid dispersion composition after the wash step is less than about 50%, less than about 40%, less than about 30%, less than about 20%, less than about 10%, less than about 5%, less than about 2%, less than about 1%, or less than about 0.5%, relative to the surfactant present in the emulsion.

In embodiments, the method further optionally includes drying the washed solid dispersion composition (or, in the absence of a wash step, the solid dispersion composition, e.g. after step (d)) to remove the anti-solvent. The drying may be performed by methods known in the art, e.g., vacuum distillation, rotary evaporation, desiccator, air drying, or vacuum oven drying. The appropriate method for removing the anti-solvent may be selected by the skilled person depending on the boiling point of the solvent and the anti-solvent, the sensitivity of the solid dispersion composition (or washed solid dispersion composition) to heat, for example. In embodiments, a nitrogen atmosphere may be used during the drying process.

A further aspect relates to the use of the solid dispersion composition and/or the washed solid dispersion composition as described herein for the preparation of a pharmaceutical composition, wherein the pharmaceutical composition preferably is as defined herein.

Therefore, an aspect of the present invention refers to: a method to prepare a pharmaceutical composition comprising a solid dispersion composition and one or more pharmaceutically acceptable excipients, wherein the solid dispersion comprises an active pharmaceutical ingredient (API) and a pharmaceutically acceptable dispersion carrier, wherein the method comprises:
(a) dissolving the API and the pharmaceutically acceptable dispersion carrier in a solvent to form a dissolved API/pharmaceutically acceptable dispersion carrier solution;
(b) combining the dissolved API/pharmaceutically acceptable dispersion carrier solution and an anti-solvent to form a combination, wherein the anti-solvent comprises a surfactant;
(c) mixing the combination to generate an emulsion with a pre-determined mean particle size range;
(d) removing the solvent from the emulsion to generate the solid dispersion composition, wherein the solid dispersion composition has the predetermined mean particle size range;
(e) formulating the solid dispersion composition with one or more pharmaceutically acceptable excipients to obtain the pharmaceutical composition.

In embodiments of the method to prepare a pharmaceutical composition, the method provides improvements over a spray-drying process, for example, the methods of the invention does not require and/or omit a pre-densification step and/or a roller compaction step. Additionally, the methods of the invention may avoid and/or omit a milling step to break the ribbons (formed by spray-drying) into dry granulates. Additional improvements include wherein the powder formed by the methods of the invention is improved in handling characteristics, where the spray dried powder may be fluffy and dusty as compared to the powder obtained by methods of the invention which are more free-flowing and less dusty. In embodiments of the method to prepare a pharmaceutical composition, steps (a) to (d) may be performed as described herein above for the method to prepare a solid dispersion composition.

In an embodiment, the methods of the invention include an embodiment where the process is “batch”, “continuous” or “semi-continuous” and/or combinations thereof. “Continuous” production is a flow production method where the materials, either dry bulk or fluids that are being processed are continuously in motion, undergoing chemical reactions or subject to mechanical or heat treatment. Raw materials are continuously fed into the system and products are continuously removed without interruption and can operate over extended periods, maintaining consistent conditions such as temperature, pressure and flow rates without distinctive beginning and end points for each production cycle. “Batch” production is a method of manufacturing in which products are made as specified groups or amounts, within a time frame. A batch can go through a series of steps in a large manufacturing process to make the final desired product. Semi-continuous manufacturing is characterized by the periodic addition of reactants or removal of products, combining features of both batch and continuous processes. Materials can be processed in batches but are linked together in a continuous sequence, allowing for ongoing production without interruptions.

In an embodiment of a semi-continuous process of the invention, an example apparatus is discussed below. The example apparatus has the following elements as seen in FIG. 16: tank 300 which can optionally comprise one or more of solvent, pharmaceutically acceptable dispersion carrier and API; tank 400 which can optionally comprise anti-solvent and surfactant; tank 300 is connected to wet mill 320 via feed line 302 with inline pump 304 and optional mass flow meter 306; and tank 400 is connected to wet mill 320 via feed line 402 with inline pump 404 and optional mass flow meter 406. Wet mill 320, optionally a high shear mixing device, can create an emulsion. Wet mill discharge line 322 is connected to solvent removal device 326 which is optionally a thin film evaporator which is connected to vacuum 332 via discharge line 328 allowing for solvent evaporation with condensate collection in distillate tank 330. The solid dispersion composition is collected in flask 336 via discharge line 334. Pump 340 and exit line 338 removes the solid dispersion composition from flask 336 to liquid/solid separation device which is optionally, a decanter 342 and/or filter 344. Solids from decanter 342 and/or filter 344 are optionally washed with water, filtered in vacuum filter 344 and dewatered using dryer 346, and dewatered solid dispersion composition may be optionally sieved in sieve 348.

Therefore, in an embodiment, the present invention includes an apparatus for conducting a continuous or semi-continuous production of a solid dispersion composition, wherein the apparatus comprises: (a) a container comprising or configured to comprise a dissolved API/pharmaceutically acceptable dispersion carrier solution in a solvent; (b) a container comprising or configured to comprise an anti-solvent/surfactant solution; (c) a mixing device fluidly connected to the containers of (a) and of (b), and configured to mix the solutions of (a) and (b) to generate an emulsion, wherein the emulsion has a pre-determined mean particle size range; (d) at least one solvent removal device fluidically connected to the mixing device wherein the at least one solvent removal device is configured to remove the solvent from the emulsion to produce the solid dispersion composition; (e) a decanter or filtration device fluidically connected downstream of the at least one solvent removal device configured to collect solid dispersion composition, wherein the decanter or filtration device is optionally configured to wash the collected solid dispersion composition with an anti-solvent; wherein the apparatus further comprises one or more fluidically connected pumps configured to deliver the solutions of (a) and (b) to the mixing device and/or deliver the emulsion to the at least one solvent removal device. In an embodiment, the at least one solvent removal device is a thin film evaporator.

The formulating step can be performed according to methods known in the art, for example, by mixing the solid dispersion composition with one or more pharmaceutically acceptable excipients, screening/delumping, pre-blending, blending and pressing to form a tablet.

The term “pharmaceutically acceptable excipient” refers to a non-toxic component that does not destroy the pharmacological activity of the compound with which it is formulated. Pharmaceutically acceptable excipients that may be used in the compositions of this invention include fillers, disintegrants, glidants, lubricants, and coating agents. The compositions may comprise further pharmaceutically acceptable excipients selected from buffers, binders, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product.

The pharmaceutical composition may contain conventional non-toxic pharmaceutically acceptable excipients. In embodiments, the one or more pharmaceutically acceptable excipients are selected from the group consisting of fillers, disintegrants, glidants, lubricants, and coating agents. In embodiments, the pharmaceutical composition comprises a filler, a disintegrant, a glidant and a lubricant. In embodiments, the pharmaceutical composition comprises a filler, a disintegrant, a glidant, a lubricant and a coating agent. It is to be understood that the pharmaceutical composition may comprise one or more excipients of each function, e.g. one or more filler, one or more disintegrants, one or more glidants, one or more lubricants, one or more coating agents.

In embodiments, the filler(s) is(are) selected from the group consisting of microcrystalline cellulose, mannitol and mixtures thereof. In embodiments, the disintegrant(s) is(are) selected from the group consisting of crosslinked sodium carboxymethyl cellulose, also denoted croscarmellose sodium, sodium bicarbonate, crospovidone, sodium starch glycolate and mixtures thereof. In certain embodiments, the disintegrant is croscarmellose sodium. In embodiments, the glidant is colloidal silicon dioxide. In embodiments, the lubricant(s) is(are) selected from the group consisting of stearyl fumarate, magnesium stearate and mixtures thereof. In certain embodiments, the lubricant is sodium stearyl fumarate.

In embodiments, the one or more pharmaceutically acceptable excipients comprise mannitol, microcrystalline cellulose, croscarmellose sodium, colloidal silicon dioxide and sodium stearyl fumarate.

In embodiments, the pharmaceutical composition comprises, consists of or consists essentially of: a solid dispersion composition comprising compound (1) as defined herein or a pharmaceutically acceptable salt thereof, a pharmaceutically acceptable dispersion carrier, mannitol, microcrystalline cellulose, croscarmellose sodium, colloidal silicon dioxide and sodium stearyl fumarate.

In certain embodiments, the pharmaceutical composition comprises a coating agent, such as when formulated as a film-coated tablet. In embodiments, the coating agent comprises film-forming agents such as polyvinyl alcohol that may be partially hydrolysed, anti-tacking agents such as talc, pigments such as titanium dioxide, glyceryl mono and dicaprylocaprate (GMDCC) and iron oxides such as iron oxide yellow, and lubricants such as sodium lauryl sulphate. Coating agents are commercially available such as under the tradename Opadry® e.g. Opadry™ AMB II yellow. In a preferred embodiment, the coating agent does not contain titanium dioxide e.g. is free of titanium dioxide.

Bulk material is generally considered to a powdery, granular or lumpy mixture that exists in a pourable form; the properties of a bulk material can be partially defined by its particle size and distribution, as well as bulk density, roughness, and moisture. The method of the present invention provides a solid dispersion composition bulk material with enhanced properties relative to a bulk material prepared by conventional spray drying, for example, a bulk material prepared by the methods of the present invention will have an increased density and/or improved flowability e.g. flow function coefficient (FFc). The advantages provide for improvements in downstream processing steps to make pharmaceutical compositions and/or formulations, for example, making tablets, filling hard capsules or sachets. The improvements to density and/or FFc result in less reliance on densification steps such as e.g. roller compaction. Further, the API in the amorphous dispersions of the invention may not require mechanical strength modifiers to allow packaging and shipping. The final dosage unit of the API prepared by the processes of the present invention may require fewer excipients than the same API prepared by more conventional processes (e.g, spray drying). The densified solid dispersion compositions of the invention are also potentially amenable to direct compression in a manufacturing process. Direct compression is a preferred manufacturing strategy due to the resulting reduced need for additional excipients. Additionally, direct compression eliminates the need for roller compaction or other granulation approaches to enable tablet formation. This reduces cost and complexity of formulation for drug products and enhances solid form stability as mechanical stress of any kind (e.g. during roller compaction, milling, forced screening) may promote recrystallization.

Bulk density is the ratio of the mass of a bulk solid to its volume which determines the space occupied by a given amount of material. Densifying, densification and similar terms in the same context refer to a method for increasing the density of a material, resulting in the material having greater density than its original density, e.g., a densified co-precipitated amorphous material, or a densified co-precipitated amorphous dispersion. The term “density” encompasses each of bulk density and tapped density.

Density (such as bulk density) of the solid dispersion compositions produced by methods of the invention may be determined by methods known in the art. Suitable for the present invention is to estimate the bulk density of the resultant powder by determining the volume of a known weight of powder. For example, the powder can be placed in a graduated cylinder or volumeter and the bulk density calculated as the mass divided by the volume. Similarly, the tapped density may be calculated by tapping or compacting the powder to remove the air gaps between particles. To generate the tapped density, a tapped density tester device which can mechanically tap the cylinder at a specified rate for a set number of taps prior to measurement. In embodiments, the tapped density is determined by adding a known mass of solid dispersion composition into a volume-measurement device, such as a graduated cylinder, and subjected to a tapped density tester and measured after a defined number of taps, for example, 1250 taps.

The density (tapped or untapped) of a solid dispersion composition produced by the processes of the invention may be increased by about 1.1× or more, about 1.2× or more, about 1.3× or more, about 1.4× or more, about 1.5× or more, about 1.75× or more, about 2× or more, about 2.5× or more, about 3× or more, about 4× or more, about 5× or more, about 6× or more, up to about 10× to 20×, relative to an equivalent solid dispersion composition which is spray-dried, or to a solid dispersion composition which is prepared without step (b) and/or (c) as defined herein. In embodiments, the tapped and/or bulk density can be between about 0.2 and about 0.8 g/cm³, between about 0.3 and about 0.7 g/cm³, between about 0.4 and 0.6 g/cm³. Alternatively, the tapped and/or bulk density can be about 0.5 g/cm³. Alternatively, the tapped and/or bulk density may be 0.2 g/cm³ or more, 0.25 g/cm³ or more, 0.3 g/cm³ or more, 0.35 g/cm³ or more, 0.4 g/cm³ or more, 0.45 g/cm³ or more, up to about 2 g/cm³. In embodiments, the bulk density (tapped and/or bulk) may be 0.15 g/cm³, 0.2 g/cm³ or more, 0.25 g/cm³ or more, 0.3 g/cm³ or more, 0.35 g/cm³ or more, 0.4 g/cm³ or more, 0.45 g/cm³ or more, up to about 2 g/cm³. In these embodiments defining specific values of tapped and/or bulk density, preferably, tapped density is determined in a tapped density tester device which taps a cylinder containing the solid dispersion composition at a specified rate for a set number of taps and bulk density is determined by dividing the mass of the solid dispersion composition by its volume. Preferably, the solid dispersion composition as defined herein has a bulk density of about 0.2 g/cm³ to about 0.5 g/cm³, wherein the bulk density is determined by dividing the mass of the solid dispersion composition by its volume.

Flow properties, also referred to as powder flow or flowability, are defined as the relative movement of a bulk of particles among neighboring particles or along the container wall surface. In other words, these terms refer to the ability of a powder to flow in a desired manner in a specific piece of equipment.

In another embodiment, flow function coefficient (FFc or ff_{c}), which is indicative of flowability, is improved over an equivalent solid dispersion composition which is spray dried, or a solid dispersion composition which is prepared without step (b) and/or (c) as defined herein. Typically, the flow function coefficient of a powder is measured as known in the art, e.g. for example, in a shear cell tester. In one embodiment, the FFc of solid dispersion compositions according to the invention may be measured using a rheometer, such as an FT4 Powder Rheometer (Freeman Technology Ltd., UK) equipped with a shear cell. In an embodiment, the flowability tests can be carried out using the FT4 Powder tester using a standard methodology (25 mm vessel at 3 kPa). The tester uses the Mohr Coulomb model to generate a linearized yield locus. The procedure of a standard shear cell has three steps: powder bed pre-compaction, pre-shearing of powder bed till steady state (monitored maximum torques is stable), and shearing until the powder yields. After repeating this standard shear cell procedure by using different normal stresses (a) to get corresponding shear stress (T) for several times, the result of above process will generate a liner fit which is yield locus. The intercept of the yield locus with the shear stress axis is powder cohesion, and the slope is the linear angle of the internal friction. The flow function coefficient (ffc) was calculated from the major principal stress (MPS) divided by unconfined yield strength (UYS). The following is an explanation of FFc values:

$${ff}_{c} < 1 , not flowing ;$$

$$1 < {ff}_{c} < 2 , very cohesive ;$$

$$2 < {ff}_{c} < 4 , cohesive ;$$

$$4 < {ff}_{c} < 10 , easy - flowing ;$$

$$10 < {ff}_{c} , free - flowing .$$

In embodiments, FFc of a solid dispersion composition produced by the processes of the invention may be increased from an FFc of less than 1 (not flowing) for the conventional spray-dried FFc (or the FFc of a solid dispersion composition which is prepared without step (b) and/or (c) as defined herein) to an FFc of greater than 1 but less than 2, an FFc of greater than 1 but less than 4, an FFc of greater than 1 but less than 10; or increased from an FFc of less than 2 (cohesive) for the conventional spray-dried FFc (or the FFc of a solid dispersion composition which is prepared without step (b) and/or (c) as defined herein) to an FFc of greater than 2 but less than 3, an FFc of greater than 2 but less than 4, an FFc of greater than 2 but less than 10. In embodiments, this FFc increase is for non-blended materials. After blending, the FFc may be improved even further for the solid dispersion compositions produced by methods of the invention relative to the conventional spray dried solid dispersion composition (or the FFc of a solid dispersion composition which is prepared without step (b) and/or (c) as defined herein). In the embodiments of this paragraph—and in any other embodiment referring to FFc—the FFc is preferably measured by shear cell testing.

The term “unit dosage size,” or “tablet size,” or some combination of those terms, refers to the mass of a formulated dosage unit required to administer a given quantity of pharmaceutical.

The disclosed compositions may be formulated into hard capsules or compressed tablets. As such, further provided herein are compressed tablets comprising any of the disclosed co-precipitated materials e.g. solid dispersion compositions. In some embodiments, compressed tablets comprising a pharmaceutical composition comprising an API dispersed in one or more stabilized excipients in accordance with the disclosure. Pharmaceutical compositions, such as tablets, preferably film-coated tablets, can be manufactured according to conventional methods known to a skilled person. In embodiments, the manufacturing process can comprise the steps of 1) manufacturing a solid dispersion composition as described herein, 2) dry granulating of the solid dispersion composition with one or more suitable excipient(s), 3) blending the granules with suitable disintegrant(s) and/or lubricant(s) and/or glidants, 4) compressing the blend into tablet cores, and 5) optionally film-coating the tablet cores.

Accordingly, provided herein is a solid dispersion composition and/or emulsion comprising an API as defined herein or a pharmaceutically acceptable salt thereof and a pharmaceutically acceptable dispersion carrier as defined herein, and optionally, a surfactant as defined herein.

Also provided herein is a solid dispersion composition and/or emulsion consisting essentially of an API as defined herein or a pharmaceutically acceptable salt thereof and a pharmaceutically acceptable dispersion carrier as defined herein, and optionally, a surfactant as defined herein. As used herein, the expressions “consists essentially of” and “consisting essentially of” have the meaning attributed to them in the art. In particular, they indicate that further components may be present, especially those further components that do not have a material effect on the characteristics of the respective dispersion, composition or formulation. Such further components may for example be residual solvents.

In embodiments, said API comprises Compound (1) or a pharmaceutically acceptable salt thereof. As used herein, the term “compound (1)” refers to the compound as defined below or a pharmaceutically acceptable salt thereof:

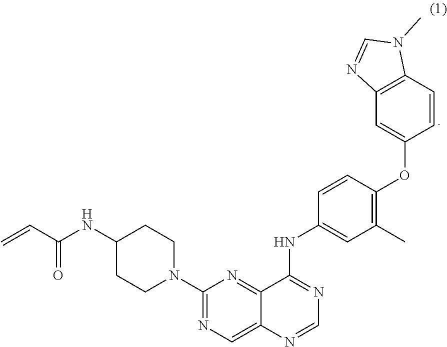 [SMILES: C=CC(=O)NC1CCN(c2ncc3ncnc(Nc4ccc(Oc5ccc6c(c5)ncn6C)c(C)c4)c3n2)CC1]

The IUPAC name of compound (1) is N-{1-[8-({3-methyl-4-[(1-methyl-1H-1,3-benzodiazol-5-yl)oxy]phenyl}amino)[1,3]diazino[5,4-d]pyrimidin-2-yl]piperidin-4-yl}prop-2-enamide. In case of discrepancy between IUPAC name and depicted formula, the formula shall prevail. Compound (1) is also known as zongertinib. Compound (1) is disclosed in WO 2021/213800 A1 as example compound I-01. WO 2021/213800 A1 describes [1,3]diazino[5,4-d]pyrimidines such as compound (1) as HER2 inhibitors and provides a synthesis procedure for compound (1). Properties of compound (1) and evidence for inhibitory effect on HER2 wild-type and YVMA kinase activity are also disclosed in WO 2021/213800 A1, which is herein incorporated by reference.

The term “compound (1)” as used herein also encompasses any tautomers and pharmaceutically acceptable salts and all solid-state forms of the compound, as well as the solvates, including hydrates and solvates of pharmaceutically acceptable salts thereof.

In embodiments, the free base of compound (1) as depicted above is used. In embodiments, pharmaceutically acceptable salt forms of compound (1) are used. The term “pharmaceutically acceptable” used herein refers to compounds, materials, compositions and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein “pharmaceutically acceptable salts” of compound (1) refers to compound (1) wherein the compound is modified by making acid or base salts thereof. The term pharmaceutically acceptable salts as used herein generally includes both acid and base addition salts. Pharmaceutically acceptable acid addition salts refer to those salts which retain the biological effectiveness and properties of the free base and which are not biologically or otherwise undesirable, formed with inorganic acids or organic acids. Pharmaceutically acceptable base addition salts include salts derived from inorganic bases or organic nontoxic bases. Examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethane sulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. In embodiments, pharmaceutically acceptable salts are selected from chloride and fumarate salts.

Pharmaceutically acceptable salts can be synthesized from compound (1) by conventional chemical methods. Generally, such salts can be prepared by reacting the free base form of compound (1) with a sufficient amount of the appropriate acid or base in water or in an organic diluent or solvent like ether, ethyl acetate, ethanol, isopropanol, or acetonitrile, or a mixture thereof.

The term “solvate” as used herein refers to an association or complex of one or more solvent molecules and compound (1). Examples of solvents include water, isopropanol, ethanol, methanol, dimethyl sulfoxide (DMSO), ethyl acetate, acetic acid, tert-butyl methyl ether, tetrahydrofuran, methylethyl ketone, N-methylpyrrolidone and ethanolamine. The term “hydrate” refers to a complex where the solvent molecule is water.

In embodiments, the API is compound (1) or a pharmaceutically acceptable salt thereof and the pharmaceutically acceptable dispersion carrier is hydroxypropyl methylcellulose acetate succinate (HPMCAS). Preferably, in these embodiments, the ratio of Compound (1) to HPMCAS can range from 5 wt % to 95 wt %. Preferably, in these embodiments, the ratio of Compound (1) is from 25 wt % to 75% wt %. Preferably, in these embodiments, the ratio of HPMCAS is from 5 wt % to 95 wt %. Preferably, in these embodiments, the ratio of HPMCAS is from 25 wt % to 75 wt %. Compound (1) can be from about 25 wt % to about 45 wt %, preferably, about 33%; and HPMCAS can be from about 55 wt % to about 75 wt %, preferably, about 66 wt %.

In embodiments, the ratio of Compound (1)/HPMCAS total weight to solvent volume is from about 0.005 g/ml to about 2 g/ml, from about 0.01 g/ml to about 1 g/ml, from about 0.05 g/ml to about 0.5 g/ml, from about 0.1 g/ml to about 0.2 g/ml. The solution can be mixed or treated in any other method known in the art to fully dissolve the Compound (1)/HPMCAS solution.

In one embodiment, the API is Compound (1) or a pharmaceutically acceptable salt thereof and the solvent is a mixture of dichloromethane (DCM) and methanol (MeOH). The relative amounts of DCM and MeOH in the mixture may vary. The relative amounts of DCM and MeOH in the mixture may vary. Preferably, the mixture comprises at least 10% v/v MeOH based on a total volume of 100% v/v of the mixture. In embodiments, the mixture comprises an excess of DCM. Still preferably, the weight:weight ratio of MeOH: DCM ranges from 5:95 to 95:5 (v/v). Preferably, MeOH and DCM are in a volume:volume ratio of approximately 10:90, 15:85, 20:80, 25:75, or 30:70. A solvent mixture of MeOH:DCM in a ratio of approximately 15:85 (v/v) is used in an embodiment of the present invention. Preferably, in these embodiments, the volume:volume ratio of DCM:MeOH ranges from 25:75 to 95:5 (v/v). Preferably, in these embodiments, DCM and MeOH are in a volume:volume ratio of approximately 25:75, 50:50, 70:30, 75:25, 80:20, 85:15 or, more preferably, 90:10. Preferably, in these embodiments, DCM and MeCOH are in a volume:volume ratio of approximately 85:15.

In embodiments, the API is Compound (1) or a pharmaceutically acceptable salt thereof and, the ratio of solvent (the solvent comprising the dissolved Compound (1) and HPMCAS) to the anti-solvent is about 1 parts solvent to a volume of anti-solvent of between about 0.5 parts to about 50 parts by volume. In embodiments, per 1 part volume of solvent, the volume of anti-solvent can be about 0.5 parts, about 1 part, about 2.5 parts, about 5 parts, about 10 parts, about 20 parts, or about 50 parts. Or, per 1 part volume of solvent, the volume of anti-solvent can be about 1 part to about 20 parts, about 2.5 parts to about 10 parts, or about 5 parts.

In embodiments, the API is Compound (1) or a pharmaceutically acceptable salt thereof and the surfactant (g) is present in the anti-solvent (ml) in an amount of about 0.5% g/ml; about 0.6% g/ml; about 0.7% g/ml; about 0.8% g/ml; about 0.9% g/ml; about 1% g/ml; about 1.1% g/ml; about 1.2% g/ml; about 1.3% g/ml; about 1.4% g/ml; about 1.5% g/ml. In other embodiments, the API is Compound (1) or a pharmaceutically acceptable salt thereof and the surfactant is present in the anti-solvent in an amount of between about 0.3% g/ml to about 3% g/ml, or about between about 0.5% g/ml to about 2% g/ml, or about 0.7% g/ml to about 1.5% g/ml, or about 0.8% g/ml to about 1.2% g/ml.

In embodiments, emulsions comprising Compound (1) or a pharmaceutically acceptable salt thereof show increased particle size stability compared to emulsions not comprising a surfactant, e.g., where particle diameter size growth is less than about 5%, about 10%, about 15%, or 20%, for a period of at least about 10 hours or more, about 18 hours or more, about 24 hours or more, about 30 hours or more, about 36 hours or more, up to about 168 hours. Alternatively, the emulsions comprising Compound (1) or a pharmaceutically acceptable salt thereof may be stable for about 10 hours to about 24 hours, from about 18 hours to about 30 hours, or from about 18 hours to about 36 hours. In embodiments, emulsions comprising Compound (1) or a pharmaceutically acceptable salt thereof exhibit minimized recrystallization shown via absence of peaks via XRPD and/or no observable crystals via PLM.

Wet milling can take place at an rpm of 3000-5000 rpm and use of medium/medium/coarse rotor-stator configuration.

In an embodiment, the API is Compound (1) or a pharmaceutically acceptable salt thereof and the solid dispersion composition is characterized by having an x-ray powder diffractogram (XRPD) comprising no diffraction peak at 2-theta angles equal or below 40.0°, when measured at a temperature in the range of from 20 to 30° C. and with Cu-Kα radiation having a wavelength of 1.54056 Å or 1.54184 Å. “Cu-Kα radiation” as used in the present invention includes Cu-Kα1 radiation and Cu-Kα1,2 radiation, wherein Cu-Kα1 radiation has a wavelength of 1.54056 Å and Cu-Kα1,2 radiation has an average wavelength of 1.54184 Å.

In another embodiment, the API is Compound (1) or a pharmaceutically acceptable salt thereof and the solid dispersion composition is characterized by having an x-ray powder diffractogram (XRPD) comprising no diffraction peak in the range of from 2.0 to 40.0°, when measured at a temperature in the range of from 20 to 30° C. and with Cu-Kα radiation having a wavelength of 1.54056 Å or 1.54184 Å.

In yet another embodiment, the API is Compound (1) or a pharmaceutically acceptable salt thereof and the solid dispersion composition is characterized by having a differential scanning calorimetry curve comprising a single glass transition temperature (T_{g}) signal, when measured with modulated differential scanning calorimetry (mDSC) with a modulation amplitude of 1° C./min and a heating rate of 3.0° C./min. Preferably, the single glass transition temperature (Tdg) signal is in the range of from 90 to 190° C., preferably of from 110 to 120° C.

In another embodiment, the emulsion of the invention comprises, consists of, or consists essentially of Compound (1) or a pharmaceutically acceptable salt thereof, a pharmaceutically acceptable dispersion carrier, and a surfactant. Preferably, in this embodiment, compound (1), the pharmaceutically acceptable salt thereof, the pharmaceutically acceptable dispersion carrier and/or the surfactant are as defined or in the amounts defined in any of the embodiments above. In embodiments of the emulsion, the pharmaceutically acceptable dispersion carrier is hydroxypropyl methylcellulose acetate succinate and Compound (1) is in an amorphous form. In embodiments of the emulsion, the surfactant is polyvinyl alcohol. In further embodiments of the emulsion, Compound (1) is present in an amount of about 20-30 wt %, based on a total weight of 100 wt % of the emulsion.

In another embodiment, the solid dispersion composition comprises, consists essentially of, or consists of Compound (1) or a pharmaceutically acceptable salt thereof and a pharmaceutically acceptable dispersion carrier. Preferably, in this embodiment, compound (1), the pharmaceutically acceptable salt thereof and the pharmaceutically acceptable dispersion carrier are as defined or in the amounts defined in any of the embodiments above. In embodiments of the solid dispersion composition, the pharmaceutically acceptable dispersion carrier is hydroxypropyl methylcellulose acetate succinate and Compound (1) is in an amorphous form. In further embodiments of the solid dispersion composition, Compound (1) is present in an amount of about 20-30 wt %, based on a total weight of 100 wt % of the emulsion. The solid dispersion composition, in embodiments, can have a bulk density of about 0.2 to about 0.5 g/cm³; or alternatively, a bulk density of about 0.3 to about 0.4 g/cm³, preferably wherein the bulk density is determined by dividing the mass of the solid dispersion composition by its volume. Alternatively, the solid dispersion composition may have a flow function coefficient (FFc) of greater than 2 in the absence of a blending step, or FFc of greater than 3 in the absence of a blending step, preferably wherein the FFc is determined by shear cell testing.

The present invention also includes a solid dispersion composition obtainable (and/or obtained) by a method of the present invention. In an embodiment, the method may comprise, consist of, or consist essentially of: the steps of dissolving compound (1) or a pharmaceutically acceptable salt thereof and a pharmaceutically acceptable dispersion carrier in a solvent to form a solution; combining the solution and an anti-solvent to form a combination, wherein the anti-solvent comprises a surfactant; mixing the combination to generate an emulsion with a pre-determined mean particle size range; and removing the solvent from the emulsion to generate the solid dispersion composition, wherein the solid dispersion composition has the predetermined mean particle size range.

In embodiments, the solid dispersion composition obtainable by a method of the invention comprises, consists of, or consists essentially of wherein pharmaceutically acceptable dispersion carrier is hydroxypropyl methylcellulose acetate succinate.

In embodiments, the solid dispersion composition obtainable by a method of the invention comprises wherein compound (1) is amorphous.

In embodiments, the solid dispersion composition obtainable by a method of the invention comprises, consists of, or consists essentially of wherein compound (1) is present in an amount of about 20-30 wt %, based on a total weight of 100 wt % of the solid dispersion.

In embodiments, the solid dispersion composition obtainable by a method of the invention comprises, consists of, or consists essentially of wherein the solid dispersion has a bulk density of about 0.2 to about 0.5 g/cm³ as measured by the ratio of the mass of an untapped sample of solid dispersion composition divided by its volume.

In embodiments, the solid dispersion composition obtainable by a method of the invention comprises, consists of, or consists essentially of wherein the solid dispersion composition has a bulk density of about 0.3 to about 0.4 g/cm³ as measured by the ratio of the mass of an untapped sample of solid dispersion composition divided by its volume.

In embodiments, the solid dispersion composition obtainable by a method of the invention comprises, consists of, or consists essentially of wherein the solid dispersion has a flow function coefficient (FFc) of greater than 2 or greater than 3, as measured by powder rheometry.

The solid dispersion composition(s), emulsion(s) and/or pharmaceutical compositions, optionally, prepared by the methods as described herein comprising Compound (1) or pharmaceutically acceptable salts thereof, can be used as medicaments. Particularly, the solid dispersion composition (s), emulsion(s) and pharmaceutical compositions as described herein can be used for the treatment and/or prevention of oncological and/or hyperproliferative disorders, in particular in anti-cancer therapy.

According to an aspect is provided the solid dispersion composition(s), emulsion(s) and/or pharmaceutical compositions, as described herein, for use as a medicament, optionally, an anti-cancer medicament. Another embodiment of the present invention is the solid dispersion composition(s), emulsion(s) and pharmaceutical compositions for treating or preventing a disease. Optionally, the disease is cancer.

In an embodiment is provided the solid dispersion composition (s), emulsion(s) and/or pharmaceutical compositions as described herein and/or as prepared by methods disclosed herein, for use in the treatment and/or prevention of a disease or a disorder modulated by HER2, particularly an oncological and/or hyperproliferative disease.

A further aspect relates to a method of treating and/or preventing a disease or disorder modulated by HER2, particularly an oncological and/or hyperproliferative disease, wherein the method comprises the step of administering the solid dispersion composition(s), emulsion(s) and pharmaceutical compositions described herein to a patient. In an embodiment, such method comprises administering to a human in need of such treatment a therapeutically effective amount of the solid dispersion composition(s), emulsion(s) and/or pharmaceutical compositions described herein.

A related aspect relates to the use of the solid dispersion composition(s), emulsion(s) and/or pharmaceutical compositions described herein in the manufacture of a medicament. An embodiment relates to the use of the solid dispersion composition(s), emulsion(s) and/or pharmaceutical compositions described herein in the manufacture of a medicament for the treatment and/or prevention of a disease or disorder modulated by HER2, particularly an oncological and/or hyperproliferative disease.

In one aspect, is provided the solid dispersion composition(s), emulsion(s) and/or pharmaceutical compositions as described herein for use in the treatment and/or prevention of a disease and/or condition, wherein the inhibition of wild type and/or mutant HER2 is of therapeutic benefit, particularly for the treatment and/or prevention of a disease and/or condition, wherein the inhibition of HER2 exon mutant protein is of therapeutic benefit. Examples of such diseases and/or conditions include, but are not limited to, oncological and/or hyperproliferative diseases such as cancer.

One aspect relates to the solid dispersion composition(s), emulsion(s) and/or pharmaceutical compositions described herein for use in the treatment and/or prevention of an oncological and/or hyperproliferative disease. A further aspect relates to the pharmaceutical composition as described herein for use in the treatment and/or prevention of an oncological and/or hyperproliferative disease. In an embodiment, the oncological and/or hyperproliferative disease is cancer.

A further aspect relates to a method of treating and/or preventing cancer, wherein the method comprises the step of administering the solid dispersion composition(s), emulsion(s) and/or pharmaceutical compositions described herein to a patient. In an embodiment, such method comprises administering to a human in need of such treatment a therapeutically effective amount of the solid dispersion composition(s), emulsion(s) and/or pharmaceutical compositions described herein.

An embodiment relates to the use of the solid dispersion composition(s), emulsion(s) and/or pharmaceutical compositions described herein in the manufacture of a medicament for the treatment and/or prevention of cancer.

In embodiments, the cancer is HER2 overexpressed, HER2 amplified and/or HER2 mutant. In embodiments, the cancer is HER2 exon 20 mutant cancer. In embodiments, the oncological and/or hyperproliferative disease is a HER2 overexpressed, HER2 amplified and/or HER2 mutant cancer. “HER2 overexpressed” as used herein refers to a cancer, where the cells of the cancer or tumor express HER2 at levels detectable by immunohistochemistry (e.g. IHC 2+ or IHC 3+) and/or methods assaying ERBB2 messenger RNA.

“HER2 amplified” as used herein refers to a cancer where the cancer or tumor cells exhibit more than 2, in particular more than 3, 4, 5, 6, 7, 8, 9 or 10, preferably more than 6, copies of the HER2 gene ERBB2.

In some embodiments, the cancer is selected from the group consisting of brain cancer, breast cancer, endocrine cancer, gastrointestinal cancer, gynecologic cancer, head and necktumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer. In addition or in alternative, said gastrointestinal cancer may be a gastrointestinal neuroendocrine tumor, preferably HER2 mutant. Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

The following are Examples of the process described herein.

### EXAMPLES

### Example 1

### Manufacture of Amorphous Solid Dispersion Compositions Comprising Compound (1) (Zongertinib)

In this Example, a solid dispersion composition was prepared containing 33 wt % compound (1) and 67 wt % of dispersion carrier HPMCAS-M (Shin-Etsu AQOAT).

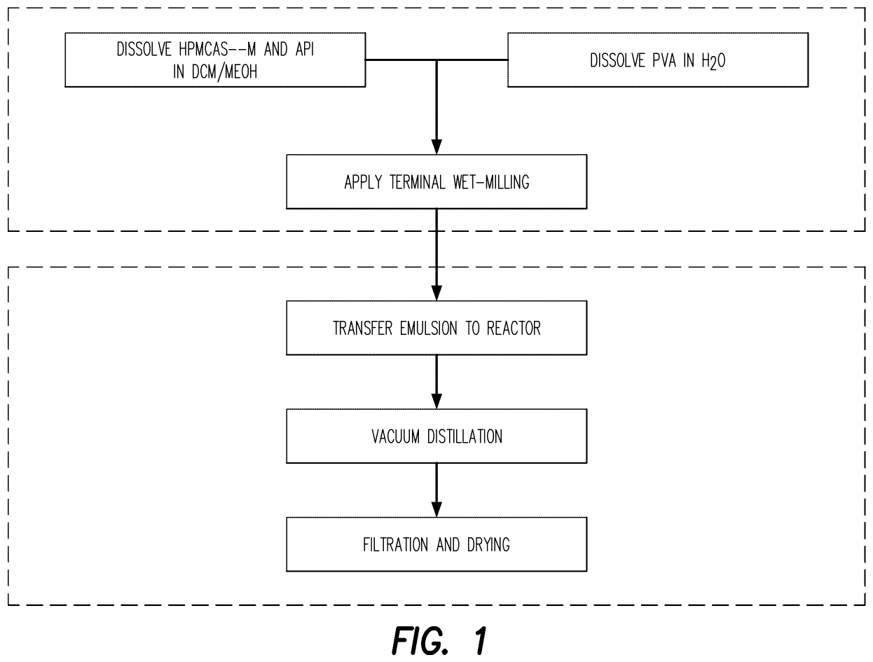

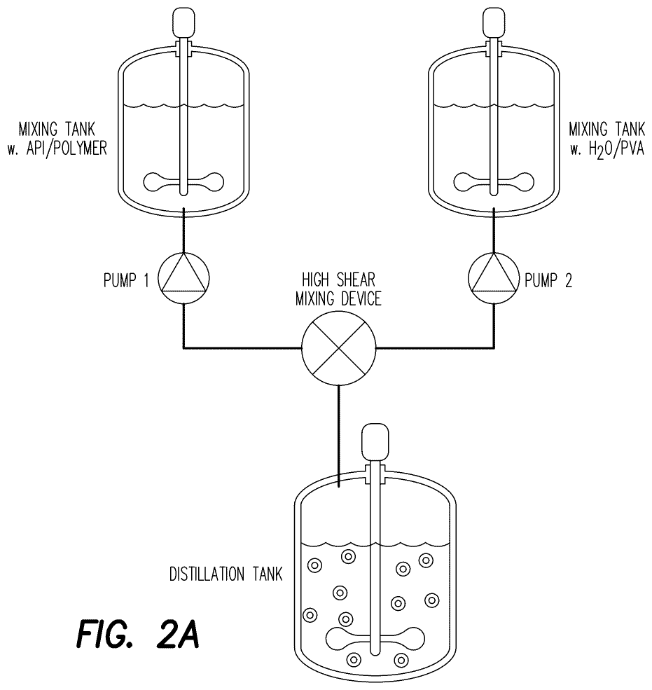

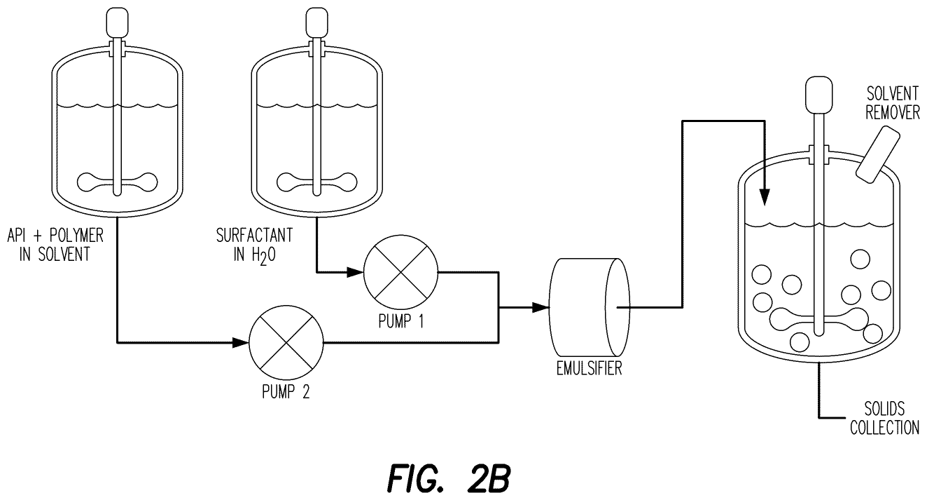

The solid dispersion composition was prepared according to the following protocol: HPMCAS-M solution was prepared by dissolving HPMCAS-M >2 h at room temperature in a premixed DCM/MeOH (85:15 (v/v)) solvent system with a HPMCAS-M content of 80 mg/mL. DCM to MeOH ratio of 85:15 (v/v) was selected based on the solubility of the compound (1) and HPMCAS-M, affording >100 mg/mL compound (1) solubility. Compound (1) (40 mg/mL) was added to the prepared HPMCAS-M solution and mixed for >2 h at room temperature to ensure the complete dissolution of the compound and achieve a desired compound to polymer ratio of 1:2 (w/w) and a solids content of 120 mg/mL total solids. The aqueous continuous phase was prepared by dissolving a surfactant, e.g. polyvinyl alcohol (PVA, e.g. Mowiol® 8-88) in water (1 wt %). The aqueous continuous phase can be filtered before use, e.g. through a 0.45 μm PTFE filter. Table 1 shows the process conditions. FIG. 1 shows a flow chart for the process flow for the manufacture of the solid dispersion compositions of compound (1). FIG. 2A shows a depiction of the equipment set up for the process flow shown in FIG. 1. FIG. 2B shows an alternative depiction of the equipment set up for the process flow shown in FIG. 1. The semi-continuous controlled API-polymer solidification process involves three main unit operations: 1) solution preparation, 2) emulsion generation, and 3) solidification (FIGS. 2A and 2B).

A high shear mixing device (IKA® Magic Lab®, Wilmington NC) with configuration as shown in Table 2 was used to generate emulsions. Two syringe pumps were used to feed the compound (1)/HPMCAS-M solution and aqueous solution simultaneously into the high shear mixing device with a flow rate of 13 mL/min and 39 mL/min, respectively. Emulsion is generated using milling conditions shown (Table 2). The emulsion prepared is transferred to a batch reactor containing H₂O (w. 1% PVA), which was subject to vacuum distillation (300-750 mBar or more specifically 500-600 mbar) at room temperature overnight to remove the organic solvents and solidify the API-polymer material. The distillation can be performed under nitrogen purge in an EasyMax or OptiMax reactor with agitation, e.g. at a speed of 250 rpm. The final suspension/slurry was subject to isolation (e.g. using a fritted funnel and/or under vacuum) and water wash followed by a secondary drying of the dispersion in a vacuum dryer of tray dryer type in collection vessels, at 25° C., 35° C. or 40° cfor 16-18 h with nitrogen purge. The material collected was a loose flowable powder, and may be variously called herein, e.g. the solid dispersion composition prepared via emulsion or the solid dispersion composition of Example 1.

Potential critical process parameters are evaluated in each unit operation, and the process conditions can be optimized considering the emulsion stability, emulsion droplet size, and physical property of the isolated materials.

Further loading the system with DCM/MeOH containing HPMCAS-M with various concentration of API also resulted in the formation of emulsion, which is visually stable after holding at room temperature for over 8 h by PLM.

TABLE 1
Process conditions

| | Weight or | | API/polymer | |
|---|---|---|---|---|
| Ingredients | Volume | | ratio | Function |
| Compound (1) | 4 | g | 1:2 | API |
| HPMCAS-M | 8 | g | | Stabilizing agent |
| DCM | 85 | mL | — | Solvent |
| MeOH | 15 | mL | — | Solvent |
| H₂O (w. 1% | 500 | mL | — | Discontinuous |
| PVA) | | | | phase |

TABLE 2
High shear conditions/emulsion generation

| IKA ® Magic Lab ®, | | |
|---|---|---|
| Wilmington NC | Rotor-stator configurations | RPM range |
| 3 stages | Medium/Coarse/Coarse | 3000 rpm |

### Example 2

### PVA Levels

Variation in PVA levels were tested. First, an emulsion system without API was prepared according to the following procedure.

HPMCAS-M solution was prepared by dissolving HPMCAS-M >2 h at room temperature in a premixed DCM/MeOH (85:15 (v/v)) solvent system with a HPMCAS-M content of 80 mg/mL, using the amounts shown in Table 1 (except that API was omitted). The aqueous continuous phase was prepared by dissolving polyvinyl alcohol (PVA, e.g. Mowiol® 8-88) in water (0, 0.5 or 1 wt. %). The aqueous continuous phase can be filtered before use, e.g. through a 0.45 μm PTFE filter.

A high shear mixing device (IKA® Magic Lab®, Wilmington NC) using parameters shown in Table 2 was used to generate emulsions. Two syringe pumps were used to feed the HPMCAS-M solution and aqueous solution simultaneously into the high shear mixing device with a flow rate of 13 mL/min and 39 mL/min, respectively. Emulsion was generated using wet milling speed of 3500 rpm.

The emulsion was subject to gentle mixing intensity and the stability [emulsion size and level of coalescence] of the emulsion via particle size growth was evaluated by polarized light microscope (PLM) using microscope model: Olympus® BX53M via visual inspection.

The emulsion without the presence of PVA was only stable for a short period of time (e.g., few minutes), while the emulsion stability was greater than 24 h with the presence of 0.5 wt. % or 1 wt. % polyvinyl alcohol. See FIG. 3, which shows emulsion stability vs. polyvinyl alcohol content. Testing without API showed the intrinsic stability of the emulsion system.

BlazeMetrics (USA) was used to monitor changes in emulsion size and the co-precipitation process during distillation. Morphological characteristics were captured using high dynamic resolution (HDR) microscopy. Emulsion size was analyzed based on chord length distribution (CLD), while the solidification of the emulsion was assessed using HDR turbidity measurements. The removal rate of DCM was measured in situ using a Raman microscope (HyperFlux™ PRO Plus, Tornado, USA), which is equipped with High Throughput Virtual Slit (HTVS™) technology.

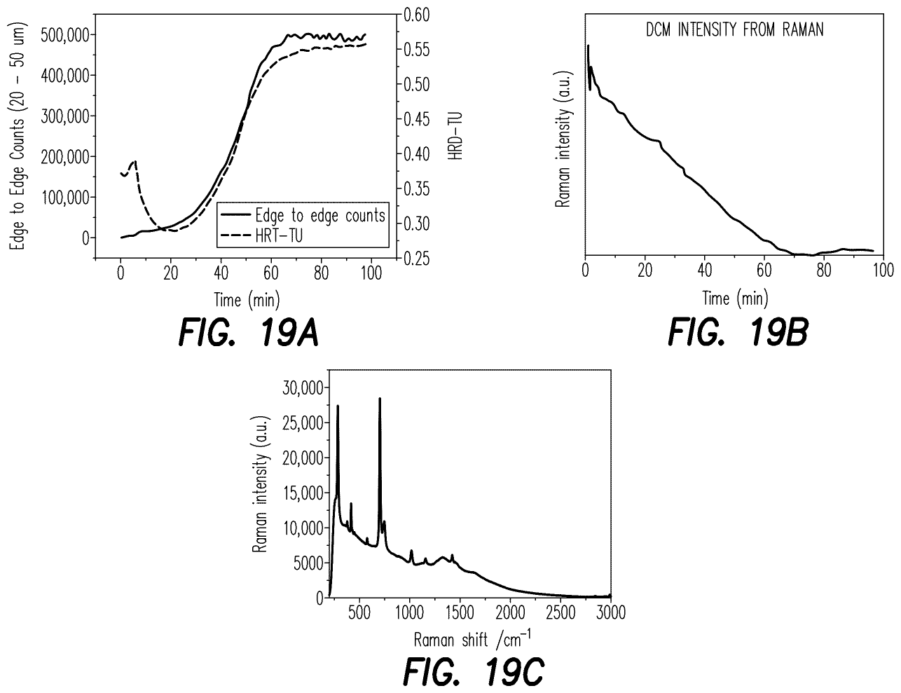

The initial emulsions processed an average size around 40-50 μm based on PLM, which reduced to ca. 20-30 um during the organic solvent removal by distillation as the emulsion continuous solidified. The solids also appeared to be denser as the organic solvent being removed, suggesting the progress of the solidification process. In addition to the off-line analysis, various advanced in-line techniques, such as BlazeMetrics and in-situ Raman, were also utilized to gain better understanding and determine the end of distillation point, which allows a more robust process control. The BlazeMetrics probe was inserted to reveal the emulsion morphology change during the process (FIG. 19A). The turbidity trend suggested the solidification smoothly proceed during the distillation and the process almost completed after ca. 1.5 h. The emulsions remained intact and maintained their spherical shape throughout the process. In addition, Raman spectrometer was applied as a powerful tool to accurately monitor the distillation rate of DCM in the emulsions and determine the end point of distillation. DCM has a distinct peak in Raman spectra, and the peak area was used to plot the removal rate of DCM (FIG. 19C). Upon applying vacuum, the DCM level gradually reduced and was completely removed after about 80 min (FIG. 19B). This trend is in line with the turbidity data from Blaze and off-line PLM images, suggesting the solidification process smoothly proceeded with the continuous removal of DCM. The end slurry was filtered and washed with H₂O prior to drying in vacuum oven at 25° C. before further characterization or formulation process.

### Example 3

### Wet-Milling Speed (Mixing Intensity)

The resultant size of the particles in emulsion/particle size distribution using different wet-milling speeds was determined.

Solid dispersion compositions were prepared according to Table 1, briefly, HPMCAS-M solution was prepared by dissolving HPMCAS-M >2 h at room temperature in a premixed DCM/MeOH (85:15 (v/v)) solvent system with a HPMCAS-M content of 80 mg/mL. The aqueous continuous phase was prepared by dissolving polyvinyl alcohol (PVA, e.g. Mowiol® 8-88) in water (1 wt. %). The aqueous continuous phase can be filtered before use, e.g. through a 0.45 μm PTFE filter.

A high shear mixing device (IKA® Magic Lab®, Wilmington NC) using parameters shown in Table 2 was used to generate emulsions. Two syringe pumps were used to feed the HPMCAS-M solution and aqueous solution (as shown in Table 1, without API) simultaneously into the high shear mixing device with a flow rate of 13 mL/min and 39 mL/min, respectively. The emulsion was generated using wet milling speed of 3000, 3750, or 4500 rpm. The generated emulsion was transferred to a batch reactor which was subject to vacuum distillation at 500 mbar for solidification for 2 h. The final slurry was washed with water and dried under room temperature for 12 h.

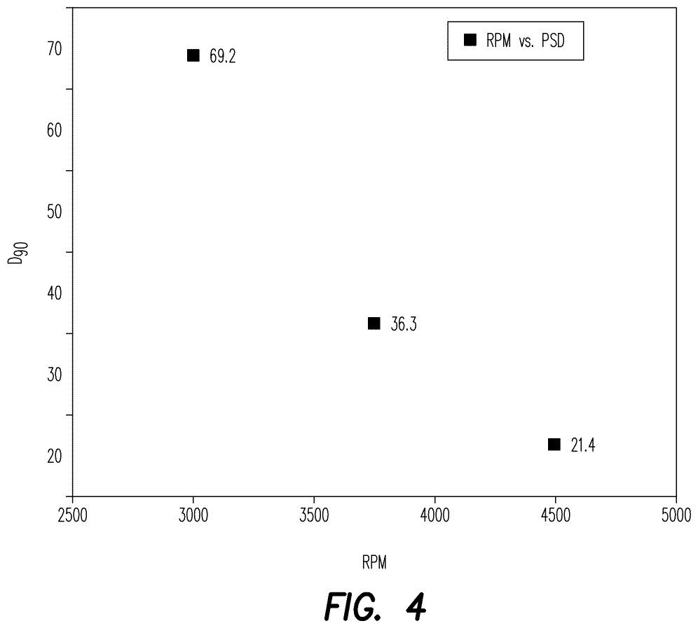

The particle size distribution (PSD) for the solid was determined using a Malvern Panalytical (Malvern, UK) MASTERSIZER 3000+ system. The parameters were as follows: particle type: spherical; Dispersant: heptane with 1% lecithin; Refractive index: 1.39; RPM: 3500. The rotor speed of the high shear device has an impact on the particle size distribution (PSD). A higher rotor speed results in lower mean particle size. Analysis was performed using Mie scattering theory for spherical particles with an estimated refractive index of 1.805 and adsorption index of 0.01 for the CAPS materials and 1.39 for the heptane. The results are summarized in FIG. 4, which shows D₉₀ in μm vs. rotor speed (RPM). The results suggest the particle size can be controlled by changing the mixing intensity in the high shear mixing device. The higher mixing intensity results in lower particle size. The D₉₀ of the dry solids decreases from 69.2 μm to 21.4 μm with the increased wet-milling speed from 3000 to 4500 rpm.

### Example 4

Characterization of Example 1 solid dispersion composition by X-Ray Powder Diffraction (XRPD), as well as characterization of additional solid dispersion compositions having different proportions of API and HPMC-AS.

The solid dispersion composition generated in Example 1, as well as solid dispersions obtained in the same manner as Example 1, but having different proportions of API and HPMC-AS (API:HPMC-AS 50:100, API:HPMC-AS 33:100, API:HPMC-AS 25:100) were subjected to XRPD analysis. XRPDs were obtained according to the following protocol: XRPD analysis was done with a D8 ADVANCE Diffractometer (Bruker, Billerica, MA). An amount of approx. 10 mg of samples of the solid dispersion composition from Example 1, was placed onto a zero-background sample disk and placed into the auto sampler of the diffractometer. Samples were analyzed using the instrument parameters described in Table 3 below.

TABLE 3
Summary of XRPD collection parameters

| Parameter | Value | |
|---|---|---|
| Θ/2-Θ | 2-35° | |
| Radiation source | Cu @1.54 Å | |
| Spin | On | |
| Voltage | 40 | kv |
| Current | 40 | mA |
| Divergent slit | 0.15 | mm |
| Holder | Silicon/zero background | |
| Step size | 0.05°/step | |
| Step rate | 0.5 | s |

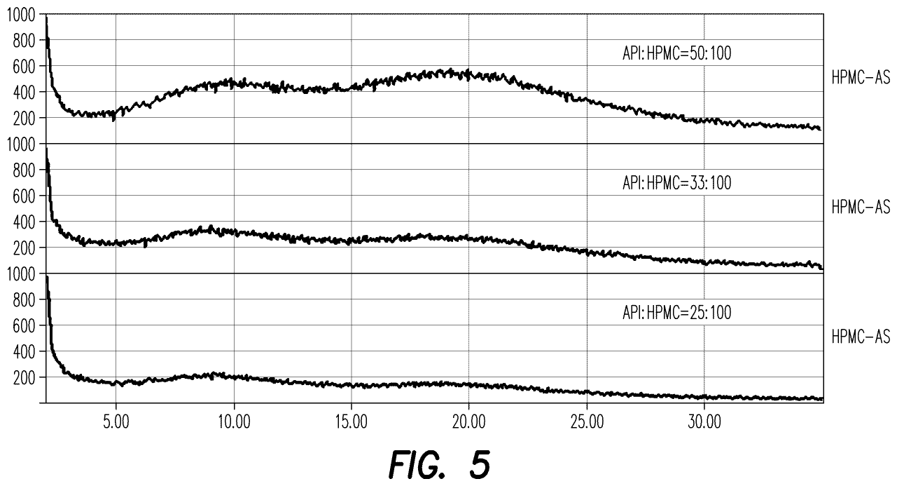

The XRPD obtained from the solid dispersion composition of Example 1 as well as API:HPMC-AS 33:100 and API:HPMC-AS 25:100, are displayed in FIG. 5. FIG. 5 indicates the absence of crystalline material in the solid dispersion compositions tested. The XRPDs of the solid dispersion compositions exhibited a lack of sharp peaks and the presence of amorphous halos. The lack of sharp diffraction peaks was indicative that the solid dispersion compositions were consistent with an amorphous form of compound (1).

### Example 5

Characterization of Example 1 Solid Dispersion Composition by Modulated Differential Scanning Calorimetry (mDSC) and Particle Size Distribution (PSD)

The solid dispersion composition of Example 1 was characterized by mDSC to determine the glass transition temperature (Tg).

mDSC was performed according to the following protocol: mDSC analysis was done on a TA Instruments/Waters (New Castle, DE) DISCOVERY DSC 2500. An amount of 2-5 mg of the solid dispersion composition of Example 1 was placed into a non-hermetic pan. A Tzero non-hermetic lid was affixed onto the pan and samples were analyzed in modulated mode in a scan range of 20 to 250° C. (unless otherwise specified) with a modulation amplitude of 1° C./min and a ramp rate (heating rate) of 3.0° C./min under N₂ flow. The Tg is determined by the midpoint of the transition.

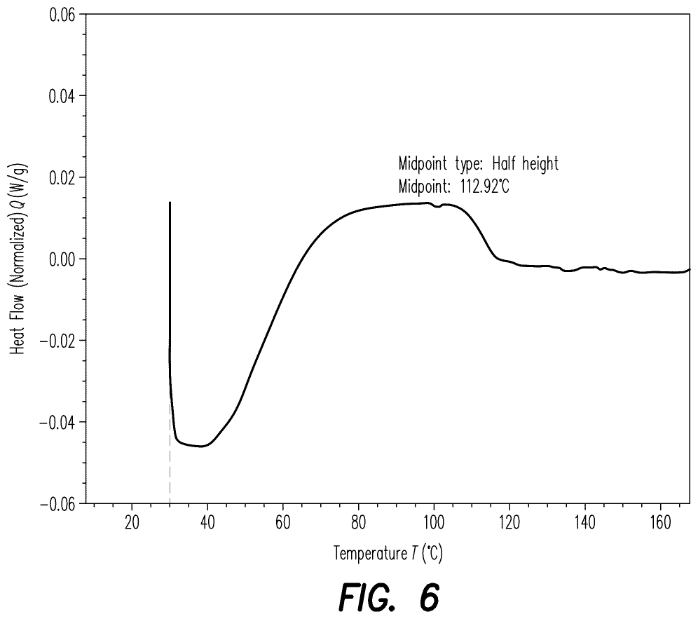

A summary of the glass transition temperatures (Tg) obtained for the solid dispersion composition of Example 1 was presented in Table 4. See also FIG. 6.

TABLE 4
Glass transition temperature (Tg) of the
solid dispersion composition of Example 1

| Formulation | Tg |
|---|---|
| 33 wt %:67 wt % compound (1):HPMCAS-M | 113° C. |

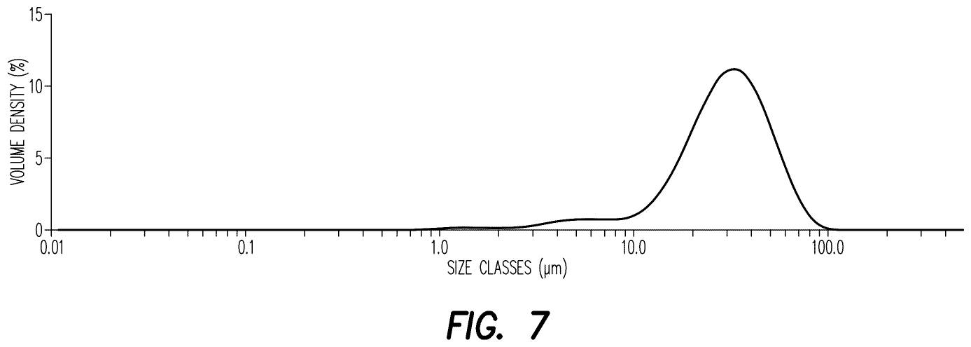

The particle size distribution (PSD) of the solid dispersion composition of Example 1 was determined using a Malvern Panalytical (Malvern, UK) MASTERSIZER 3000+ system, using conditions provided in Example 3. See FIG. 7 and Table 5.

TABLE 5
Particle size distribution (PSD) of the
solid dispersion composition of Example 1

| Sample | D10 | D50 | D90 |
|---|---|---|---|
| μm | 13.7 | 31.6 | 69.2 |

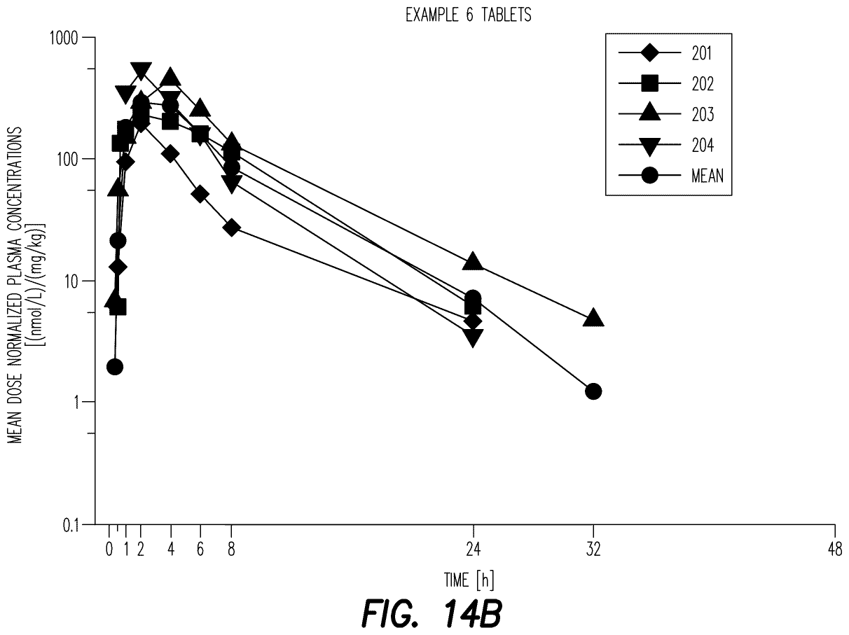

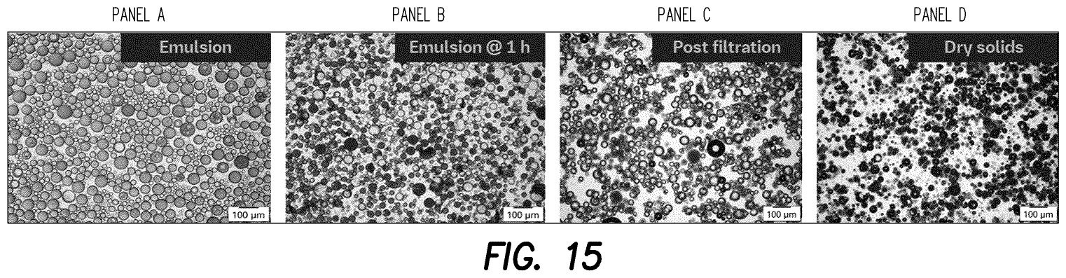

FIG. 15 shows polarized light microscope images of Panel a) Emulsion of Example 1; Panel b) Emulsion of Example 1 during solvent removal step (distillation), Panel c) solids of Example 1 after water wash (filtration), Panel d) solids after removal of the anti-solvent (vacuum-drying)(dry solids).

### Example 6

### Tableting of the Solid Dispersion Composition of Example 1

Multicomponent direct compression (DC) tablets comprising a solid dispersion composition of compound (1) prepared as described in Example 1 were prepared according to the following scheme unless otherwise specified.

The dried solid dispersion composition was mixed with microcrystalline cellulose, mannitol, and croscarmellose sodium, and the mixture of solid dispersion, fillers, and disintegrant which was screened/delumped and pre-blended. These components were screened through a mesh #18(1 mm) and blended in a Turbula blender (Glen Mills Inc., Cliffton, NJ) for 10 min at 50 rpm for a batch size of 50 g. In the next step colloidal silicon dioxide as glidant was added and mixed. Finally, sodium stearyl fumarate as lubricant was added and blended to produce a final blend.

The final blend was then compressed into tablet cores.

TABLE 6
Summary of ingredients of tablet cores

| | Quantity per | | |
|---|---|---|---|
| Ingredients | tablet (mg) | % | Function |
| compound (1) | 60.00 | 45 | active ingredient |
| hypromellose acetate | 120.00 | | dispersion carrier |
| succinate MG | | | |
| microcrystalline cellulose | 64.00 | 16 | filler |
| (Avicel PH-112 (Dupont)) | | | |
| mannitol | 132.00 | 33 | filler |
| (Pearlitol 200 SD) | | | |
| croscarmellose sodium | 12.00 | 3 | disintegrant |
| (Ac-Di-Sol (Dupont)) | | | |
| colloidal silicon dioxide | 6.00 | 1.5 | glidant |
| (Millipore) | | | |
| sodium stearyl fumarate | 6.00 | 1.5 | lubricant |
| (JRS Pharma) | | | |
| Total (tablet) | 400.00 | 100 | |

Manufacture of film-coated tablets comprising 60 mg of compound (1) can include an optional film-coating step, which was performed as outlined below.

Film-coating. A hydrophobic, protective film coating, like the ready-to-use mixture Opadry® AMB II was dispersed in purified water using a stirrer and vessel. The tablet cores were coated with the film coating suspension in a suitable pan coater to obtain film-coated tablets comprising a solid dispersion composition of compound (1) and the dispersion carrier. Alternative film coating mixtures could be used instead of Opadry® AMB II.

### Example 7

### Manufacture of Conventional Solid Dispersion by Spray Drying

In this Example, solid dispersions were prepared containing 33 wt % compound (1) and 66 wt % of dispersion carrier HPMCAS-M (Shin-Etsu AQOAT).

The solid dispersions of this Example were prepared according to the following protocol: the solid dispersion were spray dried from a spray solution composition comprising compound (1), the dispersion carrier and a DCM:MeOH (1:1 (w/w)) solvent system with a solids content of 8 wt % total solids. Solid dispersions were manufactured using a Procept 4M8TRX spray drier with 2-fluid nozzle type and 1.0 mm/1.0 mm nozzle cap/tip dimension, an inlet temperature of 85-90° C., an outlet temperature of 45-50° C., atomization at 3.0 bar, drying gas air flow rate of 0.50 m³/min, and a solution feed rate of approx. 15 g/min. Secondary drying of the dispersion was done in a vacuum dryer of tray dryer type in collection vessels, at 40° C. for 22.5 hours.

The solid dispersion of this Example was thus prepared according to WO 2024/133289.

### Example 8

### Tableting of the Solid Dispersion of Example 7

The spray-dried solid dispersion of Example 7 was mixed with portions of microcrystalline cellulose, mannitol, croscarmellose sodium, and colloidal silicon dioxide, and the mixture of solid dispersion and fillers, disintegrant and glidant was then pre-blended and screened/delumped. Sodium stearyl fumarate as lubricant was added to the pre-blend. The intragranular blend was then granulated using a roller compactor, equipped with a 1.0 mm screen. The screened dry granules were collected for subsequent final blending.

The granules were blended together with a pre-screened extragranular mixture of croscarmellose sodium and colloidal silicon dioxide in a blender. Sodium stearyl fumarate was added and blended to produce a final blend. See Table 7.

TABLE 7
Summary of ingredients of spray-dried solid dispersion tablet cores

| | | Theo- | 15 | 60 |
|---|---|---|---|---|
| | | retical | mgA | mgA |
| Component | | (%) | mg/unit | mg/unit |
| Intra- | 50% A Compound (1), HPMCAS- | 30.000 | 30.000 | 120.00 |
| granular | SDD | | | |
| | Microcrystalline cellulose | 20.000 | 20.00 | 80.00 |
| | Mannitol | 42.000 | 42.00 | 168.00 |
| | Croscarmellose Sodium (CCS) | 3.000 | 3.00 | 12.00 |
| | Colloidal Silicon Dioxide (CSD) | 1.000 | 1.00 | 4.00 |
| | Sodium Stearyl Fumarate (SFF) | 1.000 | 1.00 | 4.00 |
| | Total (of tablet core) | 97.00 | 97.0 | 388.0 |
| Extra- | Croscarmellose Sodium | 2.000 | 2.00 | 8.00 |
| granular | Colloidal Silicon Dioxide | 0.500 | 0.50 | 2.00 |
| | Sodium Stearyl Fumarate | 0.500 | 0.50 | 2.00 |
| | Total (of tablet core) | 100.00 | 100.0 | 400.0 |
| Coating | Opadry AMB II 88A120087 | 4.5/3.5 | 4.5 | 14.0 |
| | Yellow | | | |
| | Total (of tablet core) | | 104.5 | 414.0 |

The final blend was then compressed into tablet cores.

### Example 9

Dissolution profile of Example 1 solid dispersion composition according to the invention and Example 6 solid dispersion composition.

In vitro dissolution profile in phosphate buffer pH 6.8 with 0.1% (w/v) SDS between the solid dispersion composition of Example 1 and the spray-dried solid dispersion of Example 6.

A dissolution test comparison was performed comparing 60 mg of the respective materials. In vitro release testing was performed using compendial QC dissolution to assess the properties of the dosage form and to evaluate if the process parameters during manufacturing of the CAPS material have an impact on drug product quality.

Dissolution testing was performed under the conditions outlined in Table 8.

TABLE 8
Conditions for dissolution profile testing

| Apparatus: | USP II, Paddle | |
|---|---|---|
| Medium: | pH 6.8, 50 mM phosphate buffer with 0.1% | |
| | SDS | |
| Paddle rotation speed: | 75 | rpm |
| Volume: | 900 | mL |
| Temperature: | 37° | C. |

| | |
|---|---|
| Number of Replicate | 3 |
| Runs | |
| Analysis | in situ Fiber optic UV probes connected to a |
| | The Rainbow Dynamic Dissolution |
| | Monitor ® (Delphian Technology LP, Ardsley, |
| | USA (wavelength range for concentration |
| | calculation 410-415 nm with background |
| | correction) |

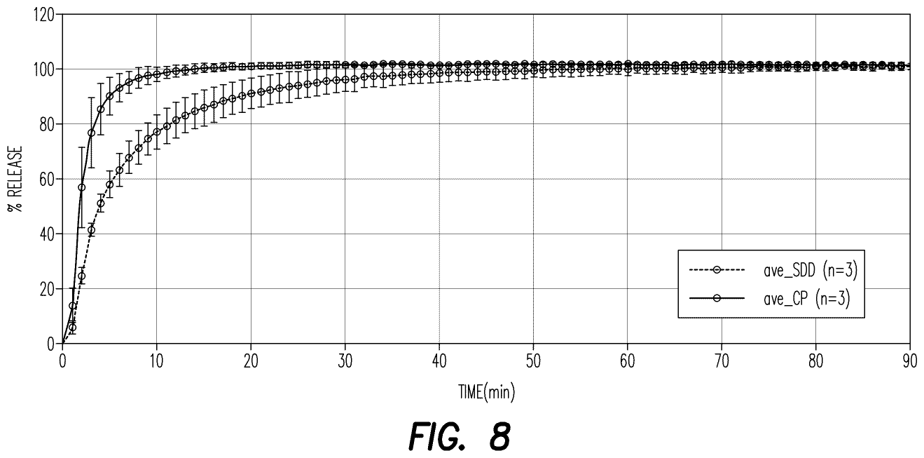

The % drug release (same as % drug dissolved) was calculated by dividing the measured drug concentration by the total amount of drug in the solid dispersion formulations (60 mg). The average drug released (n (replicates)=3) over time are shown in FIG. 8. Example 1 solid dispersion was called “Cp” (left curve) and Example 6 solid dispersion was “SDD” (right curve) in FIG. 8. The results show that both materials achieve over 85% release within 15 minutes. As can be seen, solid dispersions of Example 1 and Example 6 have similar dissolution profiles and show multiple-fold higher drug release than crystalline powder (data not shown).

### Example 10

### Flowability

Comparison for flowability of the solid dispersion composition as prepared in Example 1 versus a spray-dried bulk material prepared as described in Example 7; also described was the bulk material of Example 1 after blending.

The flow function coefficient (FFc or ff_{c}) of the respective powders was measured using an FT4 Powder Rheometer (Freeman Technology Ltd., UK) equipped with a shear cell. Flowability tests can be carried out using the FT4 Powder tester using a standard methodology (25 mm vessel at 3 kPa). The tester uses the Mohr Coulomb model to generate a linearized yield locus. The procedure of a standard shear cell has three steps: powder bed pre-compaction, pre-shearing of powder bed till steady state (monitored maximum torques is stable), and shearing until the powder yields. After pre-shear, the powder was sheared under 5 normal stresses from 0.8 kPa to corresponding pre-shear normal stresses. After repeating this standard shear cell procedure by using different normal stresses (σ) to get corresponding shear stress (τ) for several times, the result of above process will generate a liner fit which is yield locus.

The intercept of the yield locus with the shear stress axis is powder cohesion, and the slope is the linear angle of the internal friction. The flow function coefficient (ffc or FFc), which is indicative of powder flowability, was calculated from the major principal stress (MPS) divided by unconfined yield strength (UYS, stress required to initiate failure of the powder).

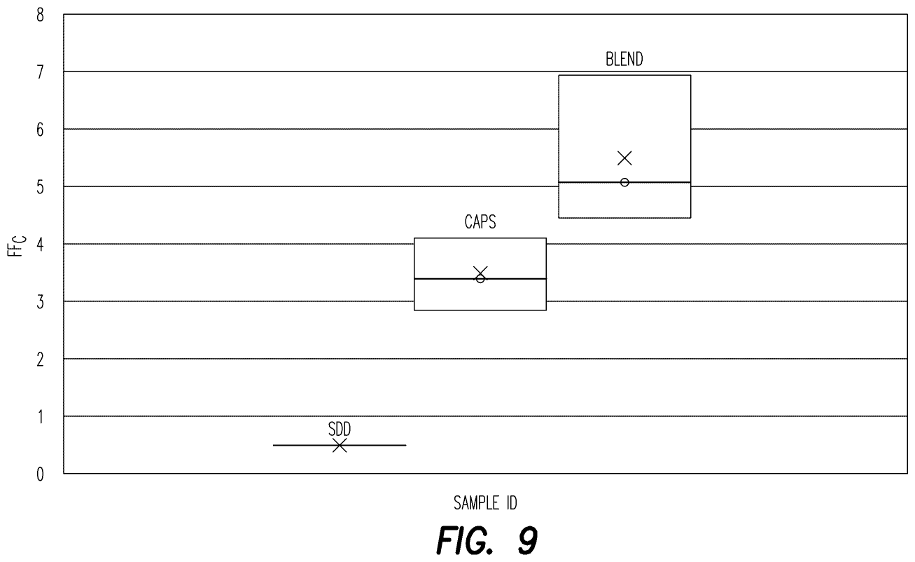

The bulk material of Example 7 had a FFc of <1, indicating the material was not flowing. The bulk material of Example 1 possesses an average FFc of 3.47 and was considered cohesive. Post blending, the blend of Example 1 material shows an average FFc of 5.5, indicating the blend was easy-flowing. See FIG. 9. See Table 9. The following was an explanation of FFc values:

$${ff}_{c} < 1 , not flowing ;$$

$$1 < {ff}_{c} < 2 , very cohesive ;$$

$$2 < {ff}_{c} < 4 , cohesive ;$$

$$4 < {ff}_{c} < 10 , easy - flowing ;$$

$$10 < {ff}_{c} , free - flowing .$$

TABLE 9
Comparison of FFc measurement of Example 1, Example 1 post final
blend, and Example 7. Post-final blend refers to after addition
of fillers, disintegrant, glidant and lubricant and blending
at 30 rpm using a 3D Turbula blender. See Example 8.

| | | | Example 1 post- |
|---|---|---|---|
| | Example 7 | Example 1 | Blend |
| Run 1 | <1 | 2.89 | 5.08 |
| Run 2 | — | 4.12 | 6.97 |
| Run 3 | — | 3.41 | 4.46 |
| Average | — | 3.47 | 5.5 |

### Example 11

### Bulk/Tapped Density

Bulk density of the powders resulting from the methods of Example 1 v. Example 7 was measured by adding ˜5 g of solids unagitated into a 10 mL graduated cylinder and the corresponding volume was recorded. The tapped density of the material was measured on a tapped density tester (VanKel, #50-1200, Cary, NC, USA) and the end volume was recorded after 1250 taps.

The bulk and tapped density of the Example 1 powder material was 0.42 and 0.47 g/cm³, respectively. On the other hand, the Example 7 powder material bulk and tapped density was 0.14 and 0.2 g/cm³, respectively.

TABLE 10
Comparison of bulk/tapped density of the
solid dispersions of Examples 1 vs. 7

| | Example 1 | Example 7 |
|---|---|---|
| Bulk density (g/cm³) | 0.42 | 0.14 |
| Tapped density (g/cm³) | 0.47 | 0.2 |

### Example 12

### API/Polymer Ratio (Assay, Content Uniformity (“CU”))

The API and HPMC-AS content of the in-process and finished tablets of Example 6 was obtained using a size exclusion chromatography (SEC)-based HPLC method. To improve the sensitivity of detecting HPMC-AS with low UV absorbance, a max-light cartridge cell (60 mm path length, 4.0 μL volume, Agilent Technologies, Santa Clara, CA USA) was used. The method demonstrated satisfactory specificity, linearity, precision and accuracy, making it suitable for measuring a range of 0.001-0.2 mg/mL API and 0.1-1 mg/mL HPMC-AS.

For Example 1 solid dispersion composition analysis, samples were analyzed using an Agilent 1260 HPLC (Agilent Technologies, Santa Clara, CA USA), equipped with a G7104C quaternary pump, a G7129C autosampler, a G7116A column compartment, and a G7117C diode array detector (DAD) that includes a max-light cartridge cell (60 mm path length, 4.0 μL volume). The DAD detector was set to operate at 350 nm for API and at 205 nm for HPMCAS. An Agilent AdvanceBio SEC column (4.6×150 mm, 1.9 μm, 200 Å, Agilent Technologies, Santa Clara, CA USA) was employed, with the column temperature maintained at 40° C. The SEC separation was performed under isocratic condition at a flow rate of 0.3 mL/min, with a mobile phase comprising a mix of 50 mM sodium phosphate buffer pH=6.8 and acetonitrile (70/30, v/v). The injection volume was set at 15 μL. A water/methanol mixture (50/50, v/v) was used as the diluent. Concentration of API in the solid dispersion composition was expected to be 0.60 mg/ml. See Table 11.

TABLE 11
Content uniformity assay of Example 1 material

| Sample | Concentration (mg/mL) |
|---|---|
| Run 1 | 0.60 |
| Run 2 | 0.55 |
| Run 3 | 0.58 |
| Average | 0.58 |
| Target | 0.60 |
| Std Dev | 0.03 |
| CV % | 4.5% |

### Example 13

### Physical Stability of Amorphous Solid Dispersion Composition of Example 1

A stress stability study was conducted at accelerated stress conditions.

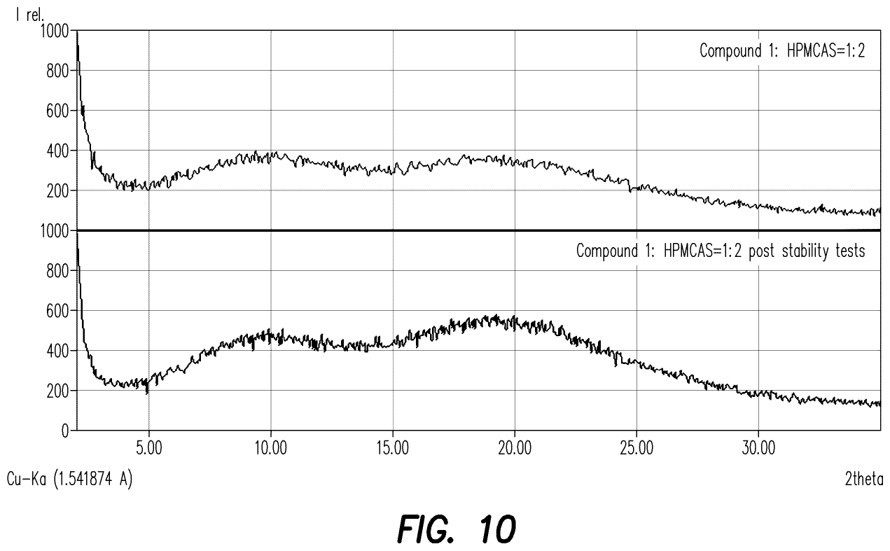

Physical stability of a bulk material prepared according to Example 1 was evaluated in an accelerated stability study. The sample was incubated at 70° C. and 75% relative humidity (RH) sealed condition in an aluminum pouch. After three weeks, samples were removed for analysis and characterized via XRPD to evaluate potential recrystallization. No changes in physical properties were observed. Diffractograms of all samples of the amorphous solid dispersion composition (diffractograms obtained as described in Example 4) remained consistent with an amorphous form of compound after three weeks at the defined storage condition. See FIG. 10, showing the XRPD pattern of the samples before (top) and after (bottom) stability tests.

### Example 14

### Predicting Drug Product Performance by Physiologically-Relevant In Vitro Testing: Tiny-TIM

The TIM is a multi-compartmental model that simulates gastro-intestinal tract (GIT) conditions. The TIM-1 system represents the upper and middle GIT and consists of four serial compartments simulating the stomach, duodenum, jejunum, and ileum. The tiny-TIM system is an abbreviated version of TIM-1, consisting of three gastric compartments and then two compartments representing the small intestine. The small intestinal compartment is connected to a polysulfone plasma filter with a pore size of ≤50 nm and a surface area of 0.3 m² to remove the compound which dissolved the gastrointestinal media used for the study. The amount of the compound in the collected filtrate per time period is considered as the fraction available for absorption from the upper gastrointestinal tract, known as the bioaccessible amount. (Ref Luo et al, Using Tiny TIM Dissolution and In Silico Simulation to Accelerate Oral Product Development of a BCS Class II Compound. AAPS Pharm. Sci. Tech, (2022) 23:185).

### Fasted and Fed State In Vitro Analysis Using Tiny TIM

For the preparation for fasted and fed state in vitro analysis using Tiny-TIM, the protocol-specific procedure provided by the TIM Company (Netherlands) for fasted and fed conditions was used. To control the physiological parameters such as variable pH values in place and time, secretion, and composition of digestive fluids, eight syringes filled with buffers and enzymes are part of Tiny-TIM. The release of these buffers and enzymes is controlled by the instrument software. The eight syringes contain as follows: gastric enzyme solution (1×) (syringe #1), pancreatin solution (3.5%) (syringe #2), water (syringe #3), bile (syringe #4), 1 M HCl (syringe #5), 1 M NaHCO₃ solution (syringe #6), small intestine electrolyte concentrate (SIES) (syringe #7), and water (syringe #8). The experiments were started by filling the gastric compartment with either fasted or fed state medium after closing the antrum region. The tablets were dropped, the cap/cover was closed, and the run was started. The pancreatin, bile, and gastric enzyme syringes were refilled during the run according to the protocol. The samples (filtrate) from a polysulfone plasma filter were collected and analyzed using HPLC. The differences between fasted and fed run conditions are listed in Table 12 and Table 13 below.

TABLE 12
Fasted and fed state conditions in Tiny-TIM

| | Fasted | Fed (high-fat meal)) |
|---|---|---|
| Starting gastric chamber pH | 3.0 | 6.5 |
| Starting small intestine (SI) pH | 7.0 | 7.0 |
| Change in gastric pH | 3.0 → ~1.8 | 6.5 → ~1.8 |
| Housekeeping wave (min) | 60 | 180 |
| pH of SI after housekeeping wave | 7.0 → 6.5 | 7.0 → 6.5 |
| Time points | 12 (4 × 15 | 12 (every 30 |
| | min, then every | min up to 360 min) |
| | 30 min) | |

TABLE 13

| | Sample | Tablet | | tTIM |
|---|---|---|---|---|
| Batch | Description | Strength | Drug load | N = 1 |
| Tablet Example 8 | Core (400 mg) | 60 mg | 15% | 60 mg dose; |
| | | | (45% DPI) | Fed |
| Tablet Example 6 | Core (400 mg) | 60 mg | 15% | 60 mg dose; |
| | | | (30% DPI) | Fed |

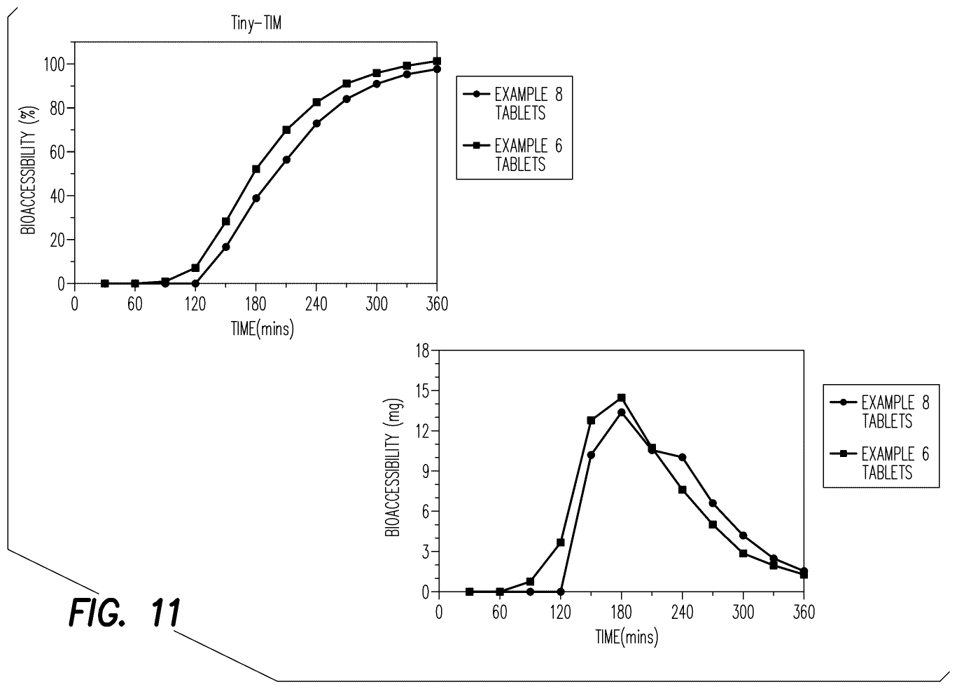

Bioaccessibility profiles for Example 6 tablet compared to Example 8 tablet in Fed State are shown in FIG. 11 as cumulative release (%) (left panel) and mg released (right panel) per timepoint. Example 6 tablet showed a faster onset of release in fed state when compared to Example 8 tablet. At 6 hrs, bioaccessible fraction is similar for both tablets, with a ratio of Example 6: Example 8 of 1.04. Bioaccessibility Max is similar for both Example 6 tablets (14.3 mg) and Example 8 (13.3 mg) tablets, and occurs at the same time (180 mins). Tablets show excellent bioaccessibility in fed state. In conclusion, performance via tiny-TIM appears similar for both formulations.

In summary, in-vitro bioaccessibility release profiles (what is available to be absorbed) from tiny-TIM were collected for both Example 8 and Example 6 tablets in the fed state. Tablets from Example 6 have similar cumulative bioaccessibility profiles, and at the end of 6 hr run, the cumulative bioaccessible fraction is similar. The bioaccessible fraction per timepoint shows similar profiles as well; maximum bioaccessibility (bioaccMax) occurs at the same time or each formulation. Example 6 tablets show a faster onset of release in the fed state compared to Example 8 tablets. See FIG. 11, showing bioaccessibility profiles shown as left, percent of drug or right, mg of drug comparing Example 8 and Example 6 tablets at same dose in tiny-TIM high fat fed state. (n=1).

### Example 15

In vitro dissolution-permeation assays of the tablets were performed using a BioFLUX™ system (Pion Inc., Billerica, MA, USA) with a Rainbow® instrument operated by AuPRO™ software (version 6.0.2.203). Data processing was performed using AuPROT™ software (version 6.0.2.203).

In the BioFLUX system, the absorption chamber was integrated with a lipophilic membrane, an overhead stirrer and fiber optic (FO) UV probe (10 mm path length) was inserted into a modified cover of a 500 mL vessel of USP II dissolution apparatus (Erweka).

The donor and absorption chambers were separated by an artificial permeation membrane. Prior to the assay, the artificial GIT-mimicking membrane was prepared by impregnating a PVDFfilter support material (polyvinylidenfluoride, 3.69 cm² open area, 0.45 μm pore size, 120 μm thickness, 70% nominal porosity) with 50 μL of 20% lecithin in dodecane lipid solution (GIT Lipid, Pion Inc., Billerica, MA, USA). The donor chambers were filled with 250 ml of biorelevant media while the absorption compartments were filled with 18 ml of acceptor sink buffer (ASB, Pion Inc., Billerica, MA, USA). The ASB was a HEPES based pH 7.4 buffer containing chemical scavengers (surfactants micelles) that allow to maintain sink conditions during the experiments. The composition of the membrane and the receiver solution were identical to the Double-Sink PAMPA (parallel artificial membrane permeation assay) model, a non-cell based method predicting of passive transcellular intestinal absorption. The temperature in both chambers was maintained at 37° C. and the paddle in the donors were rotating at 50 rpm.

The flux (J) across the membrane representing the amount (m) of material crossing one unit area (A) of the membrane per unit time (t), was calculated from the concentration-time profiles in the receiver compartments with the following equation:

$$J ( t ) = \frac{dm}{A . \cdot dt} = \frac{V}{A} \cdot \frac{dc}{dt}$$

‘A’ is the area of the membrane (3.69 cm²), ‘V’ is the volume of the receiver compartment (18 mL) and ‘dc/dt’ (μg·mL⁻¹·min⁻¹) is the slope of the concentration time profile of the drug in the receiver compartment.

The flux values were calculated by fitting the concentration-time profile in the receivers to a straight line to determine the slope and then normalizing the slope to the volume and surface area of the membrane with the following equation:

$${ff}_{c} = \frac{{σ}_{1}}{{σ}_{c}}$$

where σ₁=major principal stress, and σ_{c}=unconfined yield stress

Time intervals to calculate the flux were selected based on the apparent linearity of the concentration-time profile in the receivers, capturing the initial flux process and excluding the lag time unless otherwise is stated.

Dissolution-Permeation studies were performed using biomimetic media mimicking the composition of the canine Fasted State Simulated Intestinal Fluid (FaSSIFc). FaSSIFc media were prepared following the using pH 7.5 phosphate buffer with Biorelevant canine powder as described in the media preparation tool on the Biorelevant.com website.

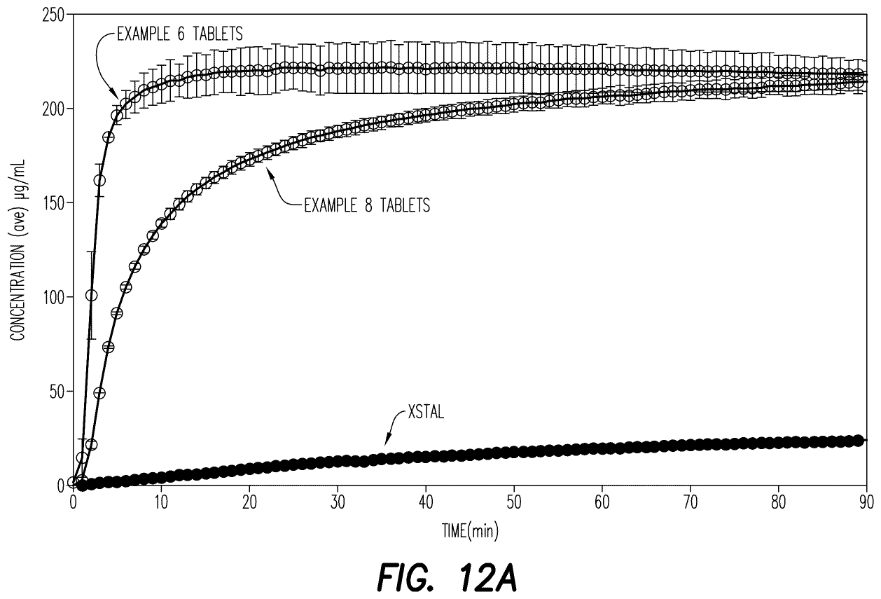

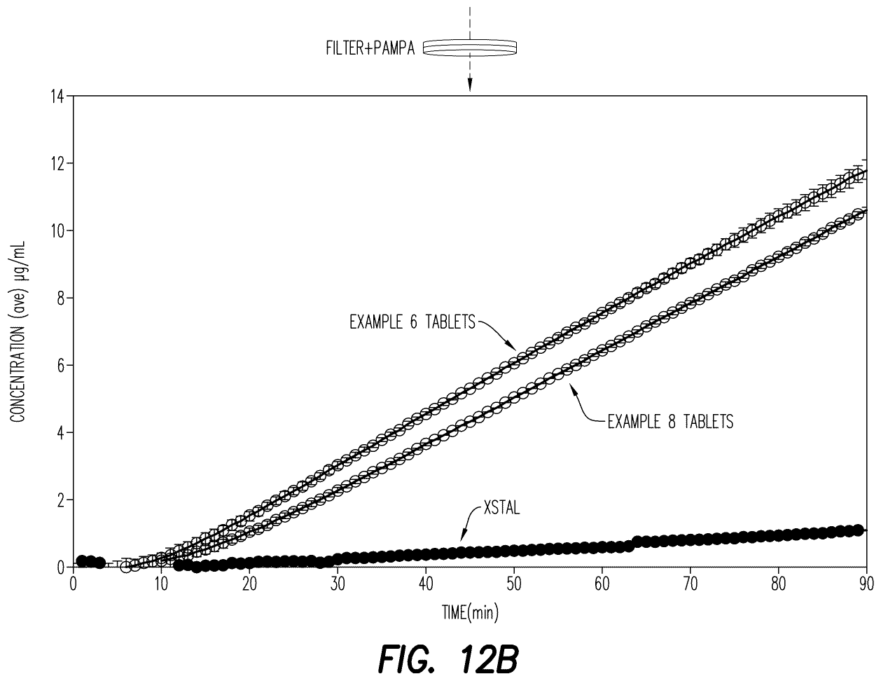

Results show fast release of compound (1) from both formulations with full drug released within 60 minutes. The faster initial released from the formulation of Example 6 could be due to the higher polymer amount. Higher drug released was achieved and maintained through formulated tablets of Example 6, compared to the crystalline API. Concentration time profiles in receivers also show that a greater amount of drug was able to permeate from tablets of Example 6 compared to crystalline material. The flux across the membrane representing the amount of drug substance crossing a unit area of the membrane per unit time, was calculated from the concentration-time profiles in the receiver compartments. Flux values for tablets of Example 8 and tablets of Example 6 in FaSSIFc (Fasted State Simulated Intestinal Fluid with cholesterol) were similar with slightly higher Flux seen for Example 6 tablets. These results suggest bioequivalence of both formulations, see Table 14. The flux ratio between the tablets of Example 6/Example 8 is 1.13, suggesting similar bioavailability. See also FIG. 12. FIG. 12 shows concentration time profile in donors (dissolution) and receivers (permeation) of crystalline API and formulated tablets from Example 6 and Example 8 in FaSSIFc. In both panels, the top line shows the tablets of Example 6, the middle line shows tablets of Example 8, and the bottom line shows crystalline API. The left panel shows dissolution and the right panel shows permeation.

TABLE 14

| | Flux Fasted canine [μg/(min*cm²)] |
|---|---|
| Crystalline API | 0.0731 |
| Tablet of Example 8 (n = 3) | 0.6533 ± 0.0208 |
| Tablet of Example 6 (n = 3) | 0.7400 ± 0.0572 |

### Example 16

### In Vivo Determination of Bioavailability

Four male beagle dogs, weighing between 14.8 to 16.0 kg, were kept individually in metabolic cages during the initial sampling period between 1 h prior to administration until 24 h after administration. Then, the animals were kept in their normal housing facility. Water was freely available to the dogs during the whole study. The dogs were fasted about 18 h prior to and during the first 2 hours after the drug administration.

Then, standardized food consisting of dry food and canned food was fed, and dog treats were offered after blood sampling. Famotidine (Famotidin-ratiopharm® 40 mg coated tablet, ratiopharm, Germany) was chosen as pre-treatment to increase the of stomach pH in the dogs up to a pH of 6 to 7. One hour after the Famotidine administration, tablets were dosed orally containing either the tablets of Example 6 or the tablets of Example 8. Both tablet prototypes had a dose strength of 60 mg (=60 mg/dog). After the administration of the Example 6 tablets, the animals were dosed orally with 60 mL of tap water via a feeding tube to ensure a defined minimum fluid volume in the stomach. About 0.5 mL of blood was collected from the cephalic vein using potassium-EDTA-coated micro tubes at the following sampling time points: 0.25, 0.5, 1, 2, 4, 6, 8, 24, 32 and 48 hours. Plasma was separated by centrifugation (5 min and 10,000×g at 4° C.), transferred into 1.5 mL Eppendorf vials, and stored at −20° C. Bioanalytical determination was performed via LC-MS/MS. The animal study was carried out in accordance with the German and European Animal Welfare Acts.

Tablets manufactured according to Example 8 with amorphous drug substance were evaluated in the first arm of the in vivo study. In the second study arm a tablet containing amorphous drug substance according to Example 6. The mean pharmacokinetic parameters after single oral administration of tablets according to Example 8 are presented below in Table 15. The mean maximum plasma concentration C(max) of tablets according to Example 8 was 989 nmol/L after oral administration, while after the Example 6 tablets, the C(max) was 1430 nmol/L. The time to reach the C(max) was in a range of 2.00-2.00 h (median: 2.00 h) for the tablet of Example 8 and ranged between 2.00-4.00 h (median: 2.00 h) for the tablet of Example 6.

The mean total exposure (AUCO inf) was 6,390 (nmol-h)/L, the mean residence time MRT(tot) was 7.13 h, and the terminal half-life t(½) was 5.75 h for the Example 8 tablets. The Example 6 tablets revealed an AUCO inf of 9,340 (nmol-h)/L, a MRT(tot) of 6.01 h, and a t(½) of 4.39 h. The sampling duration was sufficient based on the AUCrest,tz inf being 4.6% (SDD) and 1.7% (CAPS). All plasma concentrations were below the LLOQ at 48 hours after dosing.

C(max) and AUC(0-inf) were approximately 1.5-fold higher for the Example 6 tablet compared to the Example 8 tablet which served as the reference. Time to reach the maximum plasma concentration C(max) was comparable for both formulations. The mean terminal half-life t(½) and mean residence times MRT(tot) were both slightly longer for the Example 8 tablet compared to the Example 6 tablet. The peak-trough ratio (C(max)/C(32 h)) was 159 for the Example 8 tablet and 302 for the Example 6 tablet.

Based on the obtained pharmacokinetic parameters after the Famotidine pre-treatment the two tablets showed a comparable in vivo performance with a tendency of slightly better properties for the Example 6 tablet: slightly lower variability and slightly higher exposure with a comparable absorption time. However, the difference between the two tested tablets was not larger than the commonly seen biological variability.

Table 15 shows the mean pharmacokinetic parameter after single oral administration of Compound (1) as two formulations (Example 6 tablets and Example 8 tablets) to Famotidine pre-treated and fasted male Beagle dogs (n=4); data obtained by non-compartmental analysis.

TABLE 15

| Study arm: | | | 1 | 2 |
|---|---|---|---|---|
| Pre-treatment: | | | 40 mg Famotidine p.o. (−1 h) | |
| Treatment (0 h): | | | Example 8 tablet | Example 6 tablet |
| Compound (1) | | | (reference) | |
| Target dose: | [mg/animal] | | 60.0 | 60.0 |
| Parameter | Unit | Route: | p.o. | p.o. |
| C(max) | [nmol/L] | Mean (CV in %) | 989 (69.0) | 1430 (44.9) |
| C(max)/dose | [nmol/L]/[mg/kg] | Mean | 256 | 366 |
| t(max) | [h] | Median | 2.00 | 2.00 |
| | | Range | 2.00-2.00 | 2.00-4.00 |
| t(½) | [h] | Mean | 5.75 | 4.39 |
| MRT (tot) | [h] | Mean | 7.13 | 6.01 |
| AUC(rest, tz-inf) | [%] | Mean | 4.6 | 1.7 |
| AUC(0-inf) | [(nmol · h)/L] | Mean (CV in %) | 6390 (71.6) | 9340 (41.6) |
| AUC(0-inf)/dose | [(nmol · h)/L]/[mg/kg] | Mean | 1660 | 2400 |
| AUC(0-48) | [(nmol · h)/L] | Mean | 6350 | 9320 |
| C(max)/C(32 h) | | Ratio | 159 | 302 |
| C(max) | Co-precipitate/SDD | Ratio | NA | 1.45 |
| | (reference) | | | |
| AUC(0-inf) | Co-precipitate/SDD | Ratio | NA | 1.46 |
| | (reference) | | | |

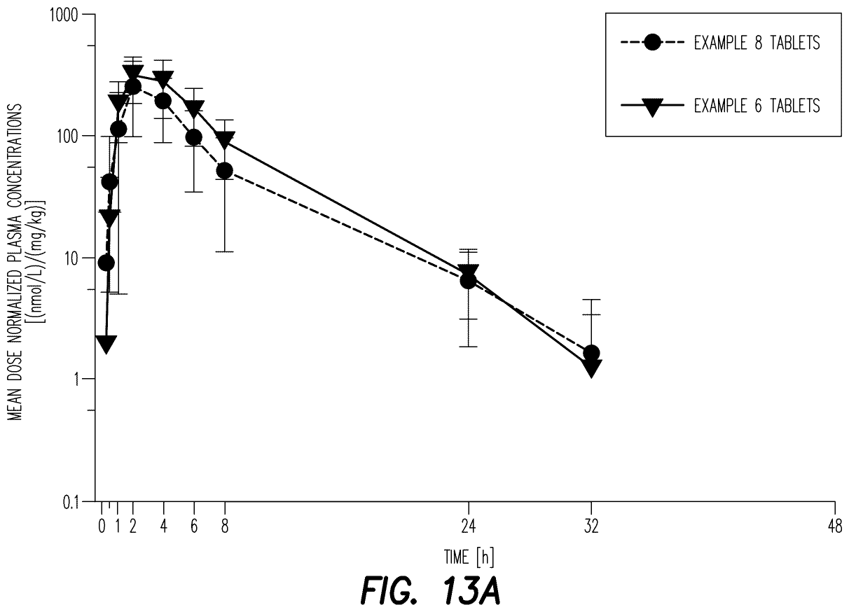

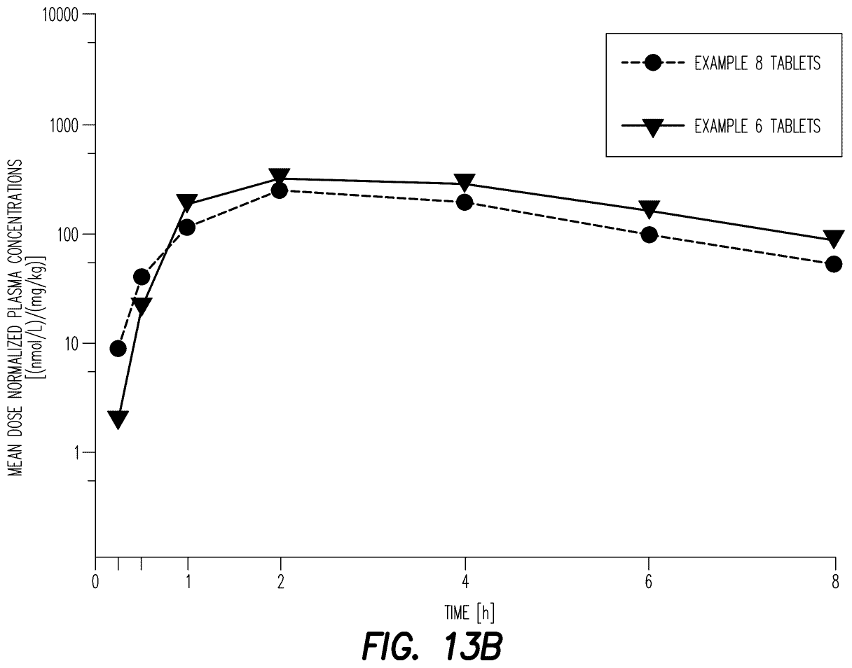

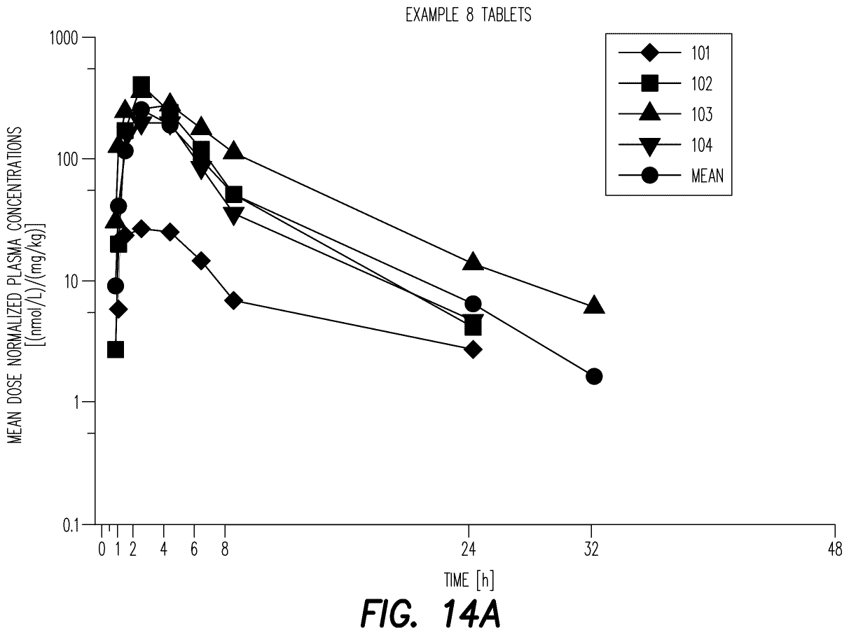

In conclusion, in-vivo PK studies were performed in 4 male beagle dogs pre-treated with Famotidine to increase their stomach up to pH 6-7. The plasma concentration profiles after single oral administration for both ASD formulations were similar with comparable pharmacokinetic parameters (Cmax, Tmax and AUC). C(max) and AUC were approximately 1.5 fold higher for Example 6 tablets compared to the Example 8 tablets while time to reach the maximum plasma concentration was comparable for both formulations. FIGS. 13, 14 show plasma concentration-time profiles (dose-normalized), for the mean (FIG. 13) and for individual dogs (FIG. 14).

### Example 17

Manufacture of amorphous solid dispersion compositions comprising compound (2) (a degrader comprising an E3 ligase binder, a linker, and a kinase inhibitor, obtained from Sigma Aldrich SML3900, CDK9 PROTAC trifluoroacetate, (E)-N-(f-(((5-(tert-butyl)oxazol-2-yl)methyl)thio)thiazol-2-yl)-1-(2-(4-(2-(2,6-dimethoxy-4-(3-oxo-3-(6-oxo-3,6-dihyropyridin-1-(2H)-yl)prop-1-en-1-yl)phenoxy)ethyl)piperazin-1-yl)-2-oxoethyl)iperidine-4-carboxamide trifluoroacetate)

In this Example, a solid dispersion composition was prepared containing 9.1 wt. % compound (2) and 90.9 wt % of dispersion carrier HPMCAS-M (Shin-Etsu AQOAT).

The solid dispersion composition was prepared according to the following protocol:

HPMCAS solution was prepared by dissolving HPMCAS-M >2 h at room temperature in a premixed DCM/MeOH (90:10 (v/v)) solvent system with a HPMCAS-M content of 8 g/L. Compound (2) (80 mg/mL) is added to the prepared HPMCAS-M solution and was mixed for >2 h at room temperature to ensure the complete dissolution of the compound and achieve a desired compound to polymer ratio of 1:10 (w/w) and a solids content of 88 mg/mL total solids. The aqueous continuous phase was prepared by dissolving a surfactant, e.g. polyvinyl alcohol in water (1 wt %)(MoWiol® 8-88). Table 12 shows the process conditions. FIG. 1 shows a flow chart for the process flow for the manufacture of the solid dispersion compositions of compound (2). FIG. 2A shows a depiction of the equipment set up for the process flow shown in FIG. 1. FIG. 2B shows an alternative depiction of the equipment set up for the process flow shown in FIG. 1. The semi-continuous controlled API-polymer solidification process involves three main unit operations: 1) solution preparation, 2) emulsion generation, and 3) solidification (FIGS. 2A and 2B). A high shear mixing device ((IKA® Magic Lab®, Wilmington NC)) with was used to generate emulsions as described in Table 2. Two syringe pumps were used to feed the compound (2)/HPMCAS-M solution and aqueous solution simultaneously into the high shear mixing device with a flow rate of 13 mL/min and 39 mL/min, respectively. Emulsion was generated using a wet milling speed of 3500 rpm (Table 13). The emulsion prepared was transferred to a batch reactor, which was subject to vacuum distillation at 500 mbar at room temperature. The final suspension was subjected to water wash followed by a secondary drying of the dispersion in a vacuum dryer of tray dryer type in collection vessels, at 40° C. for 16 h with nitrogen purge. See Table 16 and 17. The dried emulsions were dense and flowable and were suitable for tableting without additional densification steps.

TABLE 16
Process conditions

| Ingredients | Weight or Volume | | API/polymer ratio | Function |
|---|---|---|---|---|
| Compound | 0.08 | g | 1:10 | API |
| (2) | | | | |
| HPMCAS-M | 0.8 | g | | Stabilizing agent |
| DCM | 9 | mL | — | Solvent |
| MeOH | 1 | mL | — | Solvent |
| H₂O (w. 1% | 50 | mL | — | Discontinuous |
| PVA) | | | | phase |

TABLE 17
High shear wet-milling conditions/emulsion generation

| IKA magic lab | Rotor-stator configurations | RPM |
|---|---|---|
| 3 stages | Medium/Coarse/Coarse | 3500 rpm |

### Example 18

### Characterization of Example 17 Solid Dispersion Composition by X-Ray Powder Diffraction (XRPD)

XRPDs can be obtained. A comparison of the XRPDs of the solid dispersion of Example 17 versus the crystalline compound indicates the absence of crystalline material in the Example 17 samples. The XRPD of the solid dispersion composition exhibits a lack of sharp peaks and the presence of amorphous halos. The lack of sharp diffraction peaks is indicative that the solid dispersion compositions are consistent with an amorphous form of compound (2).

### Example 19

### Drug Release of Example 17 Solid Dispersion Composition

In vitro dissolution profile in phosphate buffer pH 6.8 with 0.1% SDS is obtained for the solid dispersion composition of Example 17, an amorphous compound (2) and a crystalline form of compound (2). The material of Example 17 show multiple-fold higher drug release than crystalline or amorphous forms.

### Example 20

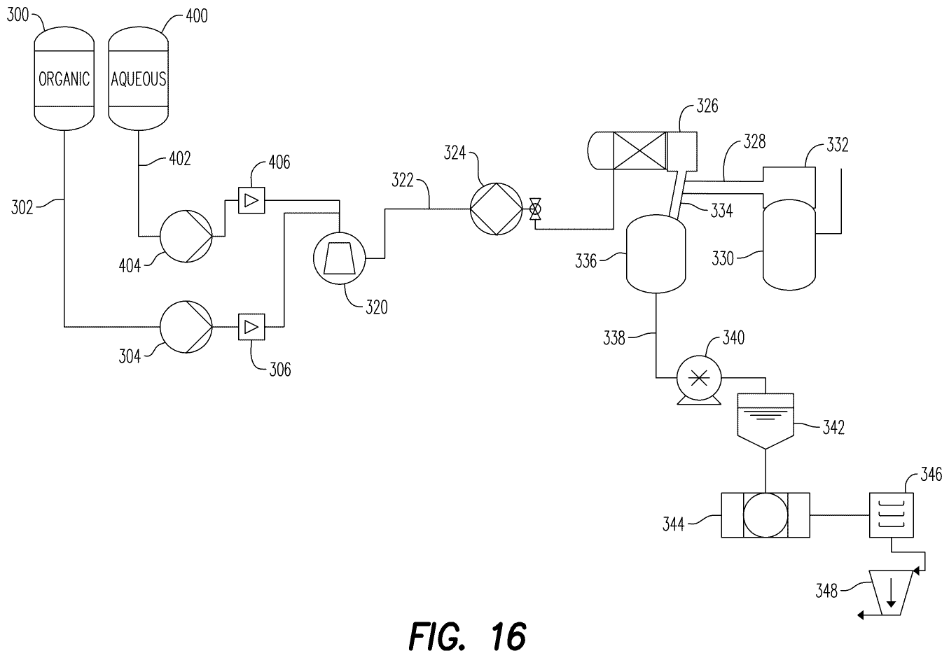

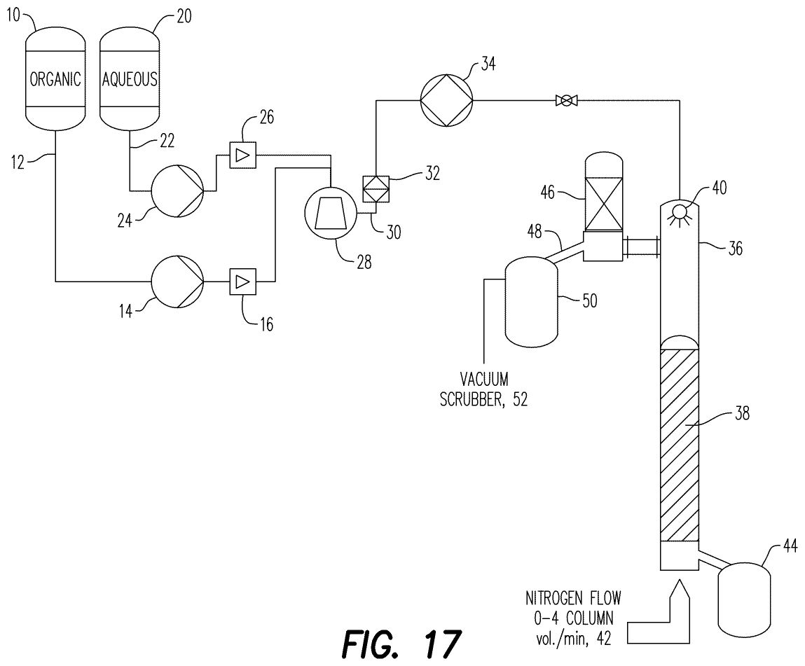

Continuous process using counter current gas flow for removal of organic solvent. Referring to FIG. 17, HPMCAS-M solution was prepared by dissolving pharmaceutically acceptable dispersion carrier HPMCAS-M >2 h at room temperature in a solvent which is a premixed DCM/MeOH (85:15 (v/v)) solvent system with a HPMCAS-M content of 80 mg/mL and agitated for not less than (NLT) 8 hours (tank 10). Compound (1) (40 mg/mL) was added to the prepared HPMCAS-M solution in tank 10 and mixed for NLT 2 h at room temperature to ensure the complete dissolution of the compound and achieve a desired compound to polymer ratio of 1:2 (w/w) and a solids content of 120 mg/mL total solids, according to Table 18.

TABLE 18

| Material | | Density/ | | | Target | |
|---|---|---|---|---|---|---|
| Description | MW | Bp° C. | Equiv | | Charge | |
| HPMCAS-M | — | — | 200% | | 400 | g |
| (Hypromellose | | | | | | |
| acetate succinate) | | | | | | |
| Methanol | 32.04 | 0.79/ | 3.75 | V | 0.75 | L |
| | | 64.7° C. | | | | |
| Dichloromethane | 84.93 | 1.33/ | 21.25 | V | 4.25 | L |
| | | 39.6° C. | | | | |
| BI 1810631 | 535.61 | — | 1.00 | | 200 | g |
| PVA (Mowiol ® 8-88) | 67,000 | — | 25% | | 50 | g |
| Water | 18.02 | 1.0/ | 25 | V | 5 | L |
| | | 100° C. | | | | |
| Water | 18.02 | 1.0/ | 10 | V | 2 | L |
| | | 100° C. | | | | |

The aqueous continuous phase (ant-solvent) was prepared by dissolving a surfactant, e.g. polyvinyl alcohol (PVA) in water (1 wt %) (tank 20). Tank 10 and 20 were 1 L tanks. Tank 10 was connected to Wet Mill 28 via Feed Line 12 with in-line pump (Fuji 3 piston pump) 14 with flow meter 16 (Brooks Coriolis mass flow meter)(optional). Tank 20 is connected to Wet Mill 28 via Feed Line 22 with in-line pump (Fuji 3 piston pump) 24 with flow meter 26 (Brooks Coriolis mass flow meter)(optional). During the process, pump 14 and 24 had a flow rate rate of 15 mL/min and 15 mL/min, respectively. Wet mill 28 was a high shear mixing device (IKA® Magic Lab®, Wilmington NC) with configuration having 3 stages, where rotor-stator configurations of medium/coarse/coarse and RPM 3000 rpm used to generate an emulsion. The emulsion discharge line 30 was transferred via peristaltic pump 34 (Ismatec peristaltic pump) at 30 ml/min to inline particle size analyzer 32 (Canty optimal particle analyzer)(optional) and then to distillation column 36. InThe distillation column 36 comprises 1-18″ and 3-12″ 60 mm jacketed columns, packed scrubber column 38, shower head 40 for emulsion feeding into distillation column 36.

Evaporated solvent is transferred to condenser 46 with distillate transferred via line 48 to distillate trap 50 via vacuum scrubber 52. Scrubber column 38 was packed with ⅝″ SS (stainless steel) metal beads to a height of 5″ then ½″ SS metal beads to a total height of 15″ (10″ of ½″ SS metal beads). Distillation column 36 nitrogen flow source tank 42 is from the bottom of column 36, and a discharge receiver 44 for distilled emulsion. Continuous vacuum distillation was performed with column jacket for column 36 temperature set to 50° C. with a vacuum of 600 torr with a nitrogen bleed from nitrogen flow source tank 42 of 2 LPM.

The distilled emulsion was collected on a Buchner funnel and washed with USP water. 2 L of water is used per 100 g of zongertinib. The washed filtrate was then subjected to secondary drying using a vacuum dryer of tray dryer type in collection vessels, at 20-25° C. for NLT 12 hours with nitrogen purge. After drying, the dried material was passed through a co-mill with a 3 mm screen. The material collected was a loose flowable powder, and may be variously called herein, e.g. the solid dispersion composition.

Particle size distribution PSD analysis shows uniform particle size distribution D10, D50 and D90 are 9.6, 21.5 and 40.7 μm, respectively, measured according to Example 5. Flowability was measured as described in Example 10. The flowability for the resultant dried solid dispersion particles (spherical particles) was assessed as 3.47. After blending the spherical particles, the flowability was 5.5. Bulk and tapped density was 0.41 and 0.46 g/cc, respectively, as determined by methods described in Example 11.

### Example 21

Kg Process Configuration with in-Line Cleaning.

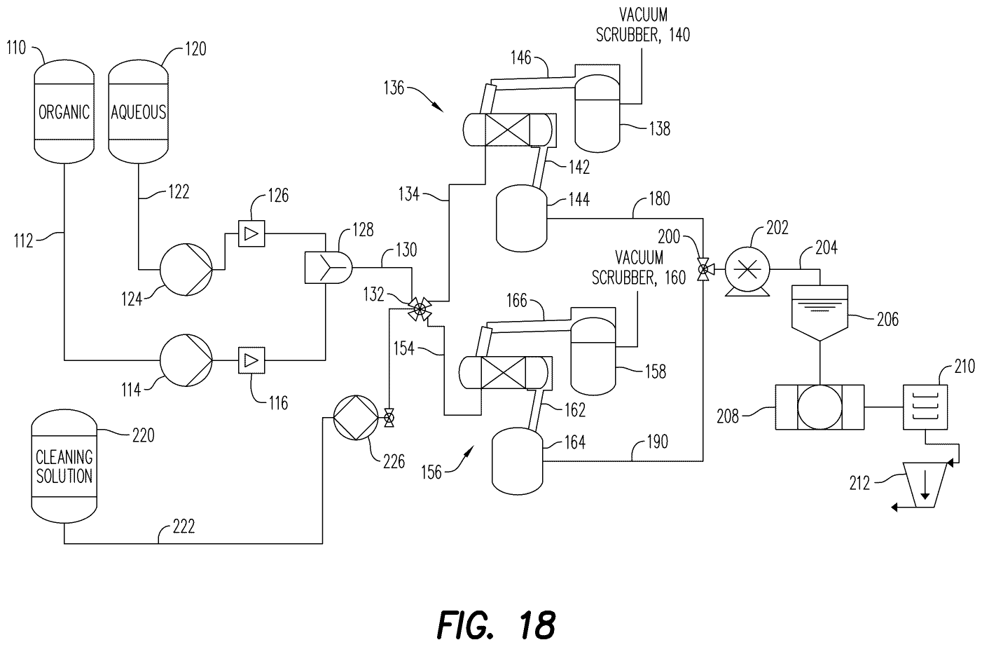

Referring to FIG. 18, tank 110 which is an agitated vessel was charged with HPMCAS-M (400 g, 200%) followed by a charge of dichloromethane/methanol (5 L, 25V; 85:15 (v/v)) at RT and the mixture was agitated until a solution formed, not less than (NLT) 8 hours. Zongertinib (200 g, 1.00 eq) was added to the HPMCAS-M in dichloromethane/methanol solution and mixed until fully dissolved. To form the anti-solvent in Tank 120, surfactant, e.g. polyvinyl alcohol (PVA) in water (1 wt %, 50 g) was dissolved in purified water (5 L, 25V) at RT and mixed until fully dissolved. Any undissolved solids were removed by polish filtration. Tank 110 and 120 were 20 L Chem Glass Reactor tanks. Tank 110 was connected to wet mill 128 via feed line 112 using in-line pump (Teledyne 2 piston pump) 114. Tank 120 was connected to wet mill 128 via feed line 122 with in-line pump d(HMI “Q” piston pump) 124. Mass flow meters 116 and 126 were downstream of pump 114 and 124, respectively. Wet mill 128 was a high shear mixing device (IKA® Magic Lab® with MK colloid head, Wilmington NC) with RPM 3000 rpm with a gap size of ˜0.9 mm was used to generate emulsion. Emulsion was discharged through line 130 to in-line selector valve 132. From selector valve 132 emulsion was directed via lines 134 or 154, respectively, to either thin film evaporator (1) 136, allowing for thin film evaporator (2) 156 to be cleaned in place, or thin film evaporator (2) 156, allowing for thin film evaporator (1) 136 to be cleaned in place. The evaporators 136 and 156 were Artisan Rototherm mini configured for counter-current operation Outlet 1½″ sanitary, distillation path >40 mm ID, jacketed at 50° C. Organic solvent from the emulsion was distilled using vacuum 140 or 160, respectively through lines 146 or 166, with condensate collected in distillation tanks 138 or 158. The distilled emulsion was collected from thin film evaporator 136 or 156 using discharge lines 142 or 162, respectively, to direct the distilled emulsion into 4 L flask 144 or 164, respectively. Evaporated emulsion was transferred via lines 180 and 190, respectively, to switching valve 200 followed by peristaltic pump (Masterflex L/S) 202 and exit line 204 to decanter 206. Solids from decanter 206, after water wash, were filtered in vacuum filter 208 and then sent to dryer 210, following by sieving in sieve 212. Pumps 114 and 124 were set to 15 ml/min and 15 ml/min and pump 204 to 30 ml/min and the wet mill 28 to 3000 rpm. The rototherms 136 and 156 were set with a vacuum of 600 torr, a jacket temperature of 45° C., and a rotor speed of 800 rpm. Emulsion was conveyed to through selector valve to either thin film evaporator feed line 134 or 154 to the respective rototherm and continuously vacuum distilled. Peristaltic pump 204 was set for 30 ml/min to convey the slurry to decanter 206. The emulsion slurry was allowed to settle in decanter 206 for not less than 30 minutes and the supernatant was decanted. The emulsion slurry solids were then resuspended in purified water and mixed to reslurry the solids (1:1 v/v relative to the volume of decanted slurry.) The slurry was then filtered in a 7 inch Buchner funnel 208 to rinse. The solid filter cake was then dried in a vacuum oven 210 at 25° C. with N₂ bleed for NLT 18 hours. Dried solids were sieved in a 1 mm sieve 212 and packaged. The thin film evaporators 136 and 156 were connected to cleaning solution tank 220 via feed line 222 and inline pump 224 via selector valve 132. The cleaning solution tank 220 was filled with a cleaning solution of MeOH:ACN:H₂0 (2:1:1). Cleaning was performed by filling the thin film evaporators 136 or 156 when not in use for the manufacturing process with the cleaning solution for 10 minutes, then rinsing with the cleaning solution for 1 minute. This process was repeated three times until the thin film evaporator 136 or 156 were clean. The material collected was a loose flowable powder, and may be variously called herein, e.g. the solid dispersion composition prepared via emulsion or the solid dispersion composition of Example 1.

Wet mill 128 was a high shear mixing device (IKA® Magic Lab® with MK colloid head, Wilmington NC) with configuration as shown in Table 19 was used to generate emulsions. Two pumps with flow control/monitoring or equivalent for the organic stream and aqueous stream were fed into the high shear mixing device and the emulsion was generated using milling conditions shown (Table 19).

TABLE 19
High shear conditions/emulsion generation
IKA ® Magic Lab ®, Wilmington NC

| Mill Head | setup | RPM range |
|---|---|---|
| Colloid Mill MK | Gap 0.7-0.9 mm | 3000 rpm |

Properties of prepared solid dispersion compositions from 13 runs is shown below in Table 20. Particle size was assessed after drying and sieving the solid dispersion composition. In all batches, appearance was yellow powder, crystal form via XRPD was amorphous, HPLC purity by area percent of zongertinib was >99% (average 99.76%), HPLC assay weight zongertinib was average 33.09 wt % (ranging from 31.49 to 33.98 wt %), water content was average 1.66% (ranging from 0.56% to 2.69%), residual methanol and residual dichloromethane was below the limits of detection. The bulk and tapped density is determined by methods described in Example 11. Particle size distribution was determined in accordance with the methods described in Example 5.

TABLE 20

| | Amount | D10 PSD | D50 PSD | D90 PSD | Bulk | Tapped |
|---|---|---|---|---|---|---|
| Batch | produced (g) | (μm) | (μm) | (μm) | Density | Density |
| 2108 | 228 | 4.92 | 17.7 | 37.2 | 0.32 | 0.44 |
| 2109 | 343 | 5.55 | 19.7 | 39.4 | 0.33 | 0.47 |
| 2110 | 259 | 5.83 | 19.5 | 38.8 | 0.33 | 0.47 |
| 2111 | 348 | 5.57 | 19.1 | 40.7 | 0.32 | 0.44 |
| 2112 | 434 | 5.92 | 20.3 | 40.2 | 0.33 | 0.46 |
| 2113 | 473 | 4.81 | 18.3 | 37.2 | 0.3 | 0.43 |
| 2114 | 434 | 4.91 | 20.4 | 38 | 0.33 | 0.46 |
| 2115 | 473 | 5.19 | 19.1 | 36 | 0.31 | 0.45 |
| 2116 | 386 | 5.99 | 20.8 | 40.5 | 0.3 | 0.41 |
| 2117 | 271 | 5.67 | 20.5 | 37.6 | 0.33 | 0.44 |
| 2119 | 474 | 4.35 | 19.5 | 36.9 | 0.32 | 0.45 |
| 2120 | 499 | 5.46 | 20 | 37.8 | 0.28 | 0.41 |
| 2121 | 424 | 4.76 | 19.6 | 36.7 | 0.31 | 0.44 |
| Average | | 5.30 | 19.58 | 38.23 | 0.32 | 0.44 |

### Example 22

### Characterization of Example 1 Solid Dispersion Composition by Thermogravimetric Analysis (TGA)

The solid dispersion composition of Example 1 was characterized by thermogravimetric analysis TGA.

TGA was performed according to the following protocol: the analysis was performed on TA discover TGA 5500 (TA Instruments, USA). Approximately 5 mg of the sample was added to a platinum pan. The samples were heated up to 250° C. at a heating rate of 3° C./min under N₂ flow.

The TGA shows ca. 0.5 wt. % weight loss, attributed to the remaining H₂O. The residual DCM and MeOH in the dried solid dispersion are not detected. The concentration of compound (1) in mother liquid is <0.002 mg/mL, suggesting the process has a high recovery yield. An HPLC-UV method was developed to determine the composition of Compound (1) and HPMACS-M. The results suggest the content of compound (1) and HPMC-AS are ca. 33% and 67%, respectively, which are consistent with the theoretical values. The PVA content measured by HPLC-CAS method shows in the dry solids is no more than 1 wt. % in the dried material.

### Example 23

### Characterization of Example 1 Solid Dispersion Composition by Scanning Electron Microscopy (SEM)

The solid dispersion composition of Example 1 was characterized by scanning electron microscopy (SEM). Scanning electron microscopy was used to reveal the surface property and morphology of the solid dispersion composition of Example 1 using a Hitachi SU5000 SEM. The samples were coated with ˜10 nm Pt using a Leica ACE-600 coater before analysis.

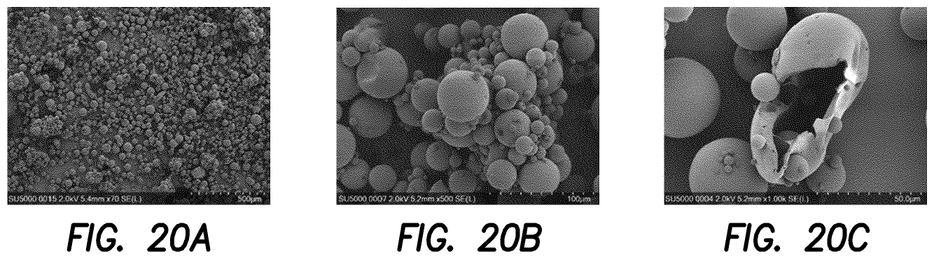

The solid dispersion composition of Example 1 possesses a uniform spherical morphology. Uniform spherical materials are observed under SEM (FIG. 20A). The high-resolution image revealed the smooth surface of the material (FIG. 20B). A closer look under SEM also revealed a hollow shaped spherical structure (FIG. 20C). This unique morphology suggests the solidification process likely to occurs first at the interface of organic and aqueous phase. As the volatile DCM leaves the emulsion droplets, both the API and HPMCAS-M started to precipitate together, leaving a hollow shaped structure. The shell of the hollow solid dispersion material also appears to be dense.

### Example 24

### Powder True Density

The true density (ρₜ) of the solid dispersion of Example 1 and the corresponding blend was determined using a helium pycnometer (AccuPyc 1330; Micromeritics, Norcross, GA) equipped with a 3.5 cm³ sample chamber. The accurately weighed sample (≈1.5 g) was placed into the measurement chamber and ρₜ was obtained from an average of ten runs on volume determination with standard deviations less than 0.0005 cm³.

### Example 25

Powder Compaction, Compressibility and Compactibility Tableting of the solid dispersion of Example 1 and corresponding formulation of Example 6 was conducted on a universal testing machine (BlueHill Universal; Instron, Norwood, MA) over a compression pressure of 25-300 MPa. A 9 mm round flat faced tooling and 15×7 oval tooling were used with target tablet weight of 300 mg and 500 mg, respectively. For the compression setup, the lower punch was kept stationary and the upper punch was descended at a speed of 0.5 mm/s. Tablets were relaxed overnight before hardness test.

Compressibility of the solid dispersion of Example 1 and the corresponding formulation of Example 6 was quantitatively expressed by the plasticity parameter, yield stress (P_{y}). The P_{y} was determined from the slope (k=1/P_{y}) of the Heckel equation below from the revised compaction pressure−porosity (ε) data shown below.

$$Heckel equation : - ln ( ε ) = k \cdot P + A$$

The inverse of the slope is related to the plasticity of the material and A is a material constant (Heckel, R. W. Density-Pressure Relationships in Powder Compaction. Trans. Metal. Soc. A. I. M. E 211, 671-675 (1961).)

The tablet tensile strength (σ)−porosity (ε) profiles of the solid dispersion of Example 1 and the corresponding formulation of Example 6 were fitted using the Ryshkewitch equation (RYSHKEWITCH, E. Compression Strength of Porous Sintered Alumina and Zirconia. J. Am. Ceram. Soc. 36, 65-68 (1953)):

$$σ = {σ}_{0} \cdot {e}^{- b ε}$$

where σ₀ often known as the intrinsic bonding strength of the powder is the compact tensile strength at zero porosity and b is an empirical constant.

The compaction property of the solid dispersion was evaluated by standard procedure using flat-faced tooling while oval shaped tooling was used to ensure manufacturability of the formulation at a target dose requiring greater tablet weight.

Tablets of Example 6 were subjected to diametric breaking on a texture analyzer (Texture Technologies Corp., Surrey, UK) at a probe speed of 0.01 mm/s and the tablet tensile strength (σ) was determined using the following equation:

$$σ = \frac{2 F}{π Dh}$$

where F, D, and h are the breaking force, tablet diameter, and thickness, respectively.

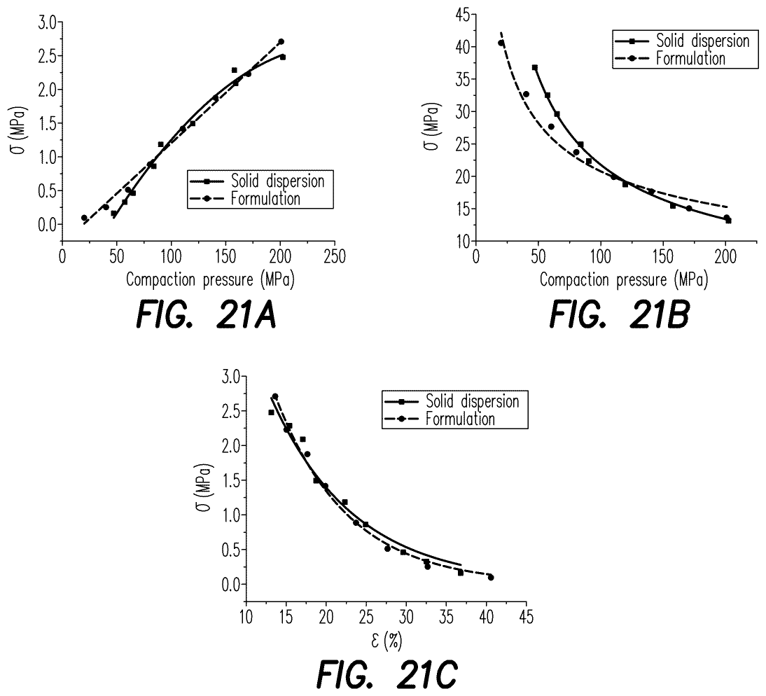

The typical compressibility-tabletability-compactibility (CTC) profiles indicated favorable compaction property of the CAPS material which showed adequate tabletability with σ>2.5 MPa (FIG. 21A). It also showed high plasticity (low P_{y}) similar to microcrystalline cellulose indicating strong plastic deformation characteristic of the solid dispersion (Table 21). For similar reason, the intrinsic bonding strength (oo) was also high favoring the compactibility of the CAPS material (Table 21).

Interestingly, the corresponding formulation followed similar trends as that of the solid dispersion material. The formulation exhibited adequate tabletability and confirming required tablet σ (>2 MPa) at a typical 15% target porosity (FIG. 21A,C). Overall, the tableting and flow data clearly implied the suitability of achieving high drug load formulation of the solid dispersion.

TABLE 21
Mechanical properties of CAPS material

| Property | (MPa) |
|---|---|
| P_{y} | 115 |
| σₒ | 13.8 |

### Example 26

### API Impurity Analysis

For API impurity analysis supporting stability studies, samples were analyzed using an Agilent 1290 Infinity II UHPLC (Agilent Technologies, Santa Clara, CA USA), equipped with a G7104A quaternary pump, a G7129B autosampler, a G7116B column compartment and a G7117A DAD. A Waters XSelect CSH C18 column (4.6×150 mm, 3.5 μm, Waters, Milford, MA USA) was used, with the column temperature set at 35° C. The mobile phase consisted of 0.1% formic acid in water (Mobile Phase A) and acetonitrile (Mobile Phase B). A linear gradient of 10-45% B over 15 min followed by 45-90% B over 10 min was applied at a flow rate of 1.0 mL/min. The injection volume was 5 μL and the UV detection was set at 245 nm. Tablet samples were first dissolved in methanol and then diluted in water/methanol mixture (50/50, v/v) for analysis.

The tablets described herein show good content uniformity, with a coefficient of variation (CV) of ≤˜5%. A reversed phase (RP) HPLC method was applied to assess the chemical stability of API in the tablets under stressed conditions. The results showed minimal API degradation, with no less than ˜99% purity in the tablets after being stored at 60° C. for 3 months.

1. A method to prepare a solid dispersion composition comprising an active pharmaceutical ingredient (API) and a pharmaceutically acceptable dispersion carrier, wherein the method comprises:
(a) dissolving the API and the pharmaceutically acceptable dispersion carrier in a solvent to form a dissolved API/pharmaceutically acceptable dispersion carrier solution;
(b) combining the dissolved API/pharmaceutically acceptable dispersion carrier solution and an anti-solvent to form a combination, wherein the anti-solvent comprises a surfactant;
(c) mixing the combination to generate an emulsion with a pre-determined mean particle size range; and
(d) removing the solvent from the emulsion to generate the solid dispersion composition, wherein the solid dispersion composition has the predetermined mean particle size range and optionally wherein the API in the solid dispersion composition is amorphous.

2. (canceled)

3. The method of claim 1, wherein the method further comprises washing the solid dispersion composition with the anti-solvent and wherein the washing step at least partially removes the surfactant.

4. (canceled)

5. The method of claim 3, wherein the method further comprises removing the anti-solvent from the solid dispersion composition.

6. The method of claim 1, wherein the method is continuous, semi-continuous or batch.

7. The method of claim 1, wherein step (d) is performed in a thin film rotary evaporator.

8. The method of claim 1, wherein the solid dispersion composition has a mean particle size range of 20 to 80 m based on polarized light microscopy, or wherein the solid dispersion composition has a bulk density of 0.2 to 0.5 g/cm³ as measured by the ratio of the mass of an untapped sample of solid dispersion composition divided by its volume, or wherein the solid dispersion composition has a flow function coefficient (FFc) of greater than 3, as measured b a powder rheometer.

9. (canceled)

10. (canceled)

11. (canceled)

12. The method of claim 1, wherein the pharmaceutically acceptable dispersion carrier is a polymer selected from the group consisting of hydroxypropyl methylcellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers.

13. (canceled)

14. The method of claim 1, wherein the solvent comprises an alcohol, a ketone, an ester, dichloromethane (DCM), chloroform, tetrahydrofuran, acetonitrile, toluene, 1,1,1-trichloroethane or mixtures thereof and wherein the anti-solvent comprises water.

15. (canceled)

16. The method of claim 1, wherein the ratio of solvent:anti-solvent is between 1:1 and 1:20 by volume.

17. The method of claim 1, wherein the surfactant comprises or is polyvinyl alcohol (PVA) in an amount of between 0.1% and 5% g/mL, and wherein optionally the PVA has a degree of hydrolysis of between 85-95% and/or the PVA has a viscosity of between 7 and 0 mPa·s.

18. (canceled)

19. The method of claim 1, wherein the API is a PROTAC such as (E)-N-(f-(((5-(tert-butyl)oxazol-2-yl)methyl)thio)thiazol-2-yl)-1-(2-(4-(2-(2,6-dimethoxy-4-(3-oxo-3-(6-oxo-3,6-dihyropyridin-1-(2H)-yl)prop-1-en-1-yl)phenoxy)ethyl)piperazin-1-yl)-2-oxoethyl)iperidine-4-carboxamide trifluoroacetate.

20. The method of claim 1, wherein the API is compound (1) 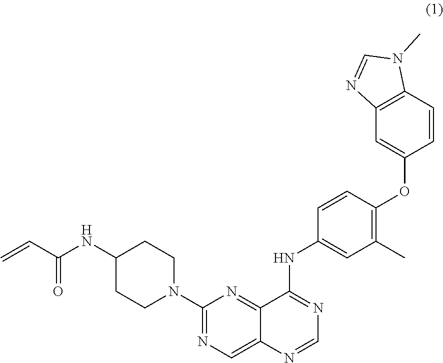 [SMILES: C=CC(=O)NC1CCN(c2ncc3ncnc(Nc4ccc(Oc5ccc6c(c5)ncn6C)c(C)c4)c3n2)CC1]
or a pharmaceutically acceptable salt thereof.

21. The method of claim 20, wherein the pharmaceutically acceptable dispersion carrier is HPMCAS, optionally wherein the solid dispersion composition comprises compound (1) in an amount in a range of from 25 wt % to 75 wt %, and HPMCAS in an amount in a range of from 25 wt % to 75 wt %, based on a total weight of 100 wt % of the solid dispersion composition.

22. (canceled)

23. (canceled)

24. The method of claim 20, wherein the solvent is a mixture of dichloromethane and methanol, optionally wherein the solvent is 85 parts by volume of dichloromethane to 15 parts by volume of methanol.

25. (canceled)

26. The method of claim 20, wherein the ratio of solvent:antisolvent is 0.2 by volume.

27. The method of ft claim 20, wherein the surfactant is polyvinyl alcohol (PVA) and the PVA is present in the anti-solvent in an amount of between 0.5% and 1.5% of the anti-solvent (g/ml), and optionally wherein the PVA has a degree of hydrolysis of between 85-95% and/or the PVA has a viscosity of between 7 and 0 mPa·s.

28. (canceled)

29. The method of claim 20, wherein the solid dispersion composition has a mean particle size range of 20 to 80 μm based on polarized light microscopy, or wherein the solid dispersion composition has a bulk density of 0.2 to 0.5 g/cm³, as measured by the ratio of the mass of an untapped sample of solid dispersion composition divided by its volume, or wherein the solid dispersion composition has a flow function coefficient (FFc) of greater than 3 as measured by a powder rheometer.

30. (canceled)

31. The method of claim 20, wherein the solid dispersion composition is characterized by having an x-ray powder diffractogram comprising no diffraction peak at 2-theta angles equal or below 40.0°, when measured at a temperature in the range of from 20 to 30° C. and with Cu-Kα radiation having a wavelength of 1.54056 Å or 1.54184 Å, or wherein the solid dispersion composition is characterized by having a differential scanning calorimetry curve comprising a single glass transition temperature signal in the range of from 110-120° C., when measured with modulated differential scanning calorimetry with a modulation amplitude of 1° C./min and a heating rate of 3.0° C./min.

32. (canceled)

33. An emulsion comprising compound (1) 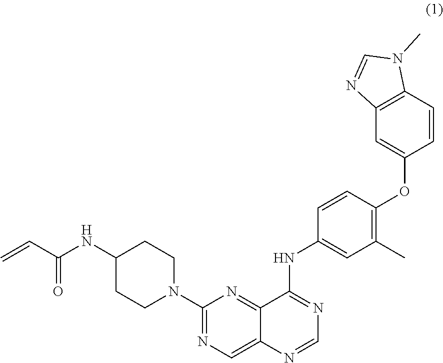 [SMILES: C=CC(=O)NC1CCN(c2ncc3ncnc(Nc4ccc(Oc5ccc6c(c5)ncn6C)c(C)c4)c3n2)CC1]
or a pharmaceutically acceptable salt thereof, a pharmaceutically acceptable dispersion carrier comprising hydroxypropyl methylcellulose acetate succinate, and a surfactant comprising PVA, and optionally wherein compound (1) is amorphous.

34. (canceled)

35. A solid dispersion composition comprising compound (1) 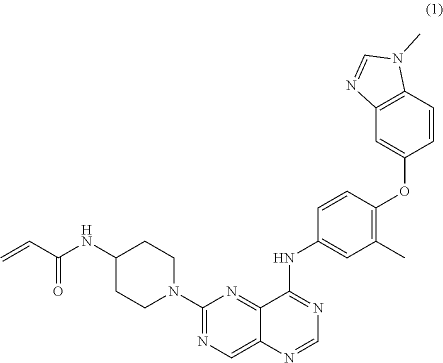 [SMILES: C=CC(=O)NC1CCN(c2ncc3ncnc(Nc4ccc(Oc5ccc6c(c5)ncn6C)c(C)c4)c3n2)CC1]
or a pharmaceutically acceptable salt thereof, and a pharmaceutically acceptable dispersion carrier comprising hydroxypropyl methylcellulose acetate succinate, optionally wherein compound (1) is amorphous, and further wherein the solid dispersion composition has a mean particle size range of 20 to 80 m based on polarized light microscopy, or wherein the solid dispersion composition has a bulk density of 0.2 to 0.5 g/cm³ or wherein the solid dispersion composition has a flow function coefficient (FFc) of greater than 2, as measured by a powder rheometer.

36. A tablet comprising the solid dispersion composition according to claim 35.

37. The solid dispersion composition according to claim 35, wherein compound (1) is present in an amount of 20-30 wt %, based on a total weight of 100 wt % of the solid dispersion composition.

38. (canceled)

39. (canceled)

40. A solid dispersion composition obtainable by a method comprising the steps of:
(a) dissolving compound (1): 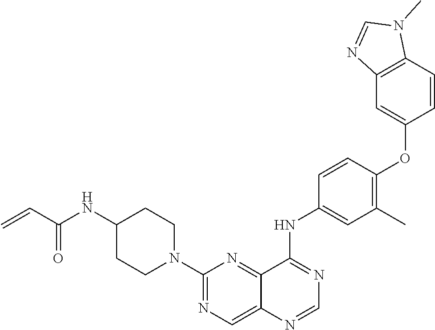 [SMILES: C=CC(=O)NC1CCN(c2ncc3ncnc(Nc4ccc(Oc5ccc6c(c5)ncn6C)c(C)c4)c3n2)CC1]
or a pharmaceutically acceptable salt thereof and a pharmaceutically acceptable dispersion carrier comprising hydroxypropyl methylcellulose acetate succinate in a solvent comprising dichloromethane/methanol to form a solution;
(b) combining the solution and an anti-solvent comprising water to form a combination, wherein the anti-solvent comprises a surfactant comprising polyvinyl alcohol;
(c) mixing the combination to generate an emulsion with a pre-determined mean particle size range;
(d) removing the solvent from the emulsion to generate the solid dispersion composition, wherein the solid dispersion composition has a predetermined mean particle size range.

41. (canceled)

42. (canceled)

43. (canceled)

44. An apparatus for conducting a continuous or semi-continuous production of a solid dispersion composition, wherein the apparatus comprises:
(a) a container (300, 10, 110) comprising a dissolved API/pharmaceutically acceptable dispersion carrier solution in a solvent;
(b) a container (400, 20, 120) comprising an anti-solvent/surfactant solution;
(c) a mixing device (320, 28, 128) fluidly connected to the containers of (a) and of (b), and configured to mix the solutions in the containers (a) and (b) to generate an emulsion, wherein the emulsion has a pre-determined mean particle size range;
(d) at least one solvent removal device (36, 326, 136, 156) fluidically connected to the mixing device (320, 28, 128) wherein the at least one solvent removal device (36, 326, 136, 156) is configured to remove the solvent from the emulsion to produce the solid dispersion composition;
(e) a decanter or filtration device (342, 206) fluidically connected downstream of the at least one solvent removal device (36, 326, 136, 156) configured to collect the solid dispersion composition, wherein the decanter or filtration device (342, 206) is optionally configured to wash the collected solid dispersion composition with an anti-solvent;
wherein the apparatus further comprises one or more fluidically connected pumps (404, 304, 24, 14, 124, 114, 324, 34) configured to deliver the solutions of (a) and (b) to the mixing device (320, 28, 128) and/or deliver the emulsion to the at least one solvent removal device (36, 326, 136, 156).

45. The apparatus of claim 44, wherein the at least one solvent removal device (326, 136, 156) is a thin film evaporator.

46. The apparatus of claim 44, wherein the API is compound (1).

47. The apparatus of claim 46, wherein the pharmaceutically acceptable dispersion carrier is HPMCAS and wherein the weight ratio of compound (1) HPMCAS in the solid dispersion composition is of 1:2; or wherein the solvent is a mixture of dichloromethane and methanol with a ratio of 85 parts dichloromethane to 15 parts methanol v/v; or wherein the anti-solvent is water, and wherein the ratio of solvent:anti-solvent is 0.2 by volume; or wherein the surfactant is polyvinyl alcohol (PVA) and the PVA is present in the anti-solvent in an amount of between 0.5% and 1.5% of the anti-solvent (g/ml).

48. (canceled)

49. (canceled)

50. (canceled)
